SEQUENCE LISTING (158 sequences)

SEQ ID NO: 1 - DNA, 372 bp, Homo sapiens
caggtgcagctgcaggagtcgggcccaggactggtgaagccttcggagaccctgtccctcacctgcactgtctctggtgactccatcagtagttactattggacctggatccggcagcccccagggaagggactggagtggattgggtatatcttttacagggggggcaccacctacaacccctccctcaagagtcgagtcaccatatcagtagacacgtccaagaaccagttctccctgaagttgaggtctgtgaccgccgcagacacggccgtgtattactgtgcgaggggagacgatcttttagtggtgacaagtgtctactggtacatcgatctctggggccgtggcaccctggtcaccgtctcctca

SEQ ID NO: 2 - protein, 124 aa, Homo sapiens
QVQLQESGPGLVKPSETLSLTCTVSGDSISSYYWTWIRQPPGKGLEWIGYIFYRGGTTYNPSLKSRVTISVDTSKNQFSLKLRSVTAADTAVYYCARGDDLLVVTSVYWYIDLWGRGTLVTVSS

SEQ ID NO: 3 - DNA, 24 bp, Homo sapiens
ggtgactccatcagtagttactat

SEQ ID NO: 4 - protein, 8 aa, Homo sapiens
GDSISSYY

SEQ ID NO: 5 - DNA, 21 bp, Homo sapiens
atcttttacagggggggcacc

SEQ ID NO: 6 - protein, 7 aa, Homo sapiens
IFYRGGT

SEQ ID NO: 7 - DNA, 54 bp, Homo sapiens
gcgaggggagacgatcttttagtggtgacaagtgtctactggtacatcgatctc

SEQ ID NO: 8 - protein, 18 aa, Homo sapiens
ARGDDLLVVTSVYWYIDL

SEQ ID NO: 9 - DNA, 360 bp, Homo sapiens
caggtgcagctggtggagtctgggggaggcgtggtccagcctggggggtccctgagactctcctgtgcagcgtccggattcaccttcagtggctatggcatgcactgggtccgccaggctccaggcaaggggctggagtggatggcagttatatggtatgatggaagtaatgattactatccagactccgtgaagggccgattcaccatctccagagacaattccaagaacacgctgtatctgcaaatgaacagcctgagagtcgaggacacggctgtgtattactgtgcgcgagatgcgtgggacctactacgttcctttgactactggggccagggaaccctggtcaccgtctcctca

SEQ ID NO: 10 - protein, 120 aa, Homo sapiens
QVQLVESGGGVVQPGGSLRLSCAASGFTFSGYGMHWVRQAPGKGLEWMAVIWYDGSNDYYPDSVKGRFTISRDNSKNTLYLQMNSLRVEDTAVYYCARDAWDLLRSFDYWGQGTLVTVSS

SEQ ID NO: 11 - DNA, 24 bp, Homo sapiens
ggattcaccttcagtggctatggc

SEQ ID NO: 12 - protein, 8 aa, Homo sapiens
GFTFSGYG

SEQ ID NO: 13 - DNA, 24 bp, Homo sapiens
atatggtatgatggaagtaatgat

SEQ ID NO: 14 - protein, 8 aa, Homo sapiens
IWYDGSND

SEQ ID NO: 15 - DNA, 39 bp, Homo sapiens
gcgcgagatgcgtgggacctactacgttcctttgactac

SEQ ID NO: 16 - protein, 13 aa, Homo sapiens
ARDAWDLLRSFDY

SEQ ID NO: 17 - DNA, 363 bp, Homo sapiens
caggtgcagctggtggagtctgggggaggcgtggtccagcctgggaggtccctgagagtctcttgtgtagtgtctggattcaccttcagcagctttggcatgcattgggtccgccaggctccagacaaggggctggagtgggtggcagttatatggtatgatggaagtaatgattactattcagactccgtgaagggccgattcaccatctccagagacaattccaagaacacgctgtttctacaaatgaaccgcctgagagccgaagacacggctgtttattactgtgcgcgagctaataactggaaccgttttgatgcctttgatctctggggccaagggacaatggtcaccgtctcttca

SEQ ID NO: 18 - protein, 121 aa, Homo sapiens
QVQLVESGGGVVQPGRSLRVSCVVSGFTFSSFGMHWVRQAPDKGLEWVAVIWYDGSNDYYSDSVKGRFTISRDNSKNTLFLQMNRLRAEDTAVYYCARANNWNRFDAFDLWGQGTMVTVSS

SEQ ID NO: 19 - DNA, 24 bp, Homo sapiens
ggattcaccttcagcagctttggc

SEQ ID NO: 20 - protein, 8 aa, Homo sapiens
GFTFSSFG

SEQ ID NO: 21 - DNA, 24 bp, Homo sapiens
atatggtatgatggaagtaatgat

SEQ ID NO: 22 - protein, 8 aa, Homo sapiens
IWYDGSND

SEQ ID NO: 23 - DNA, 42 bp, Homo sapiens
gcgcgagctaataactggaaccgttttgatgcctttgatctc

SEQ ID NO: 24 - protein, 14 aa, Homo sapiens
ARANNWNRFDAFDL

SEQ ID NO: 25 - DNA, 372 bp, Homo sapiens
gaggtgcagctggtggagtctgggggaggcttggttcagcctggagggtccctgagactctcctgtgcagcctctggattcatcttcagtaattatgaaatgaactgggtccgccaggctccagggaagggactggaatggatttcatacattactagtagtggtaatatgaaatattacgcagactctgtgaagggccgattcaccatctccagagacaacgacaagaattcactgtatctgcaaatgagtagtctgagagtcgaggacacggctgtttattattgtgtgagaggagggcgatttttggagtggttgacctactacgttatggtcgtctggggccaagggaccacggtcaccgtctcctca

SEQ ID NO: 26 - protein, 124 aa, Homo sapiens
EVQLVESGGGLVQPGGSLRLSCAASGFIFSNYEMNWVRQAPGKGLEWISYITSSGNMKYYADSVKGRFTISRDNDKNSLYLQMSSLRVEDTAVYYCVRGGRFLEWLTYYVMVVWGQGTTVTVSS

SEQ ID NO: 27 - DNA, 24 bp, Homo sapiens
ggattcatcttcagtaattatgaa

SEQ ID NO: 28 - protein, 8 aa, Homo sapiens
GFIFSNYE

SEQ ID NO: 29 - DNA, 24 bp, Homo sapiens
attactagtagtggtaatatgaaa

SEQ ID NO: 30 - protein, 8 aa, Homo sapiens
ITSSGNMK

SEQ ID NO: 31 - DNA, 51 bp, Homo sapiens
gtgagaggagggcgatttttggagtggttgacctactacgttatggtcgtc

SEQ ID NO: 32 - protein, 17 aa, Homo sapiens
VRGGRFLEWLTYYVMVV

SEQ ID NO: 33 - DNA, 360 bp, Homo sapiens
caggtgcagctggtggagtctgggggaggcgtggtccagcctgggaggtccctgagactctcctgtgcagtgtctggattcaccttcagtagctatggcatgcactgggtccgccaggctccaggcaaggggctggagtgggtggcaaatatttggtatgatggaactaatgattactatccatactccgtgaagggccgattcaccatctccagagacaattcccagaacacactatatctgcaaatgaacagcctgagagccgaggacacggctgtatattactgtgcgagagaggacttcattaactaccggtcttttgactattggggccagggaaccctggtcaccgtctcctca

SEQ ID NO: 34 - protein, 120 aa, Homo sapiens
QVQLVESGGGVVQPGRSLRLSCAVSGFTFSSYGMHWVRQAPGKGLEWVANIWYDGTNDYYPYSVKGRFTISRDNSQNTLYLQMNSLRAEDTAVYYCAREDFINYRSFDYWGQGTLVTVSS

SEQ ID NO: 35 - DNA, 24 bp, Homo sapiens
ggattcaccttcagtagctatggc

SEQ ID NO: 36 - protein, 8 aa, Homo sapiens
GFTFSSYG

SEQ ID NO: 37 - DNA, 24 bp, Homo sapiens
atttggtatgatggaactaatgat

SEQ ID NO: 38 - protein, 8 aa, Homo sapiens
IWYDGTND

SEQ ID NO: 39 - DNA, 39 bp, Homo sapiens
gcgagagaggacttcattaactaccggtcttttgactat

SEQ ID NO: 40 - protein, 13 aa, Homo sapiens
AREDFINYRSFDY

SEQ ID NO: 41 - DNA, 372 bp, Homo sapiens
caggtgcagctggtggagtctgggggaggcgtggtccagccagggacgtccctgagactctcctgtgtcgcgtctggattcaccttcagaaattttggaatgcactgggtccgccaggctccaggcaaggggctggagtgggtggcaaatatatggtttgacggaagtaatgagaactatgtcgagtccattcagggccgattcaccatctccagagacaattccaagaacacactgaatctgcagatgaacagcctgagagccgaggactcggctgtctattactgtgtgagagagggaatcctaggaactactaatccttatgatgcttttgatgtctggggccaagggacaatggtcaccgtctcttca

SEQ ID NO: 42 - protein, 124 aa, Homo sapiens
QVQLVESGGGVVQPGTSLRLSCVASGFTFRNFGMHWVRQAPGKGLEWVANIWFDGSNENYVESIQGRFTISRDNSKNTLNLQMNSLRAEDSAVYYCVREGILGTTNPYDAFDVWGQGTMVTVSS

SEQ ID NO: 43 - DNA, 24 bp, Homo sapiens
ggattcaccttcagaaattttgga

SEQ ID NO: 44 - protein, 8 aa, Homo sapiens
GFTFRNFG

SEQ ID NO: 45 - DNA, 24 bp, Homo sapiens
atatggtttgacggaagtaatgag

SEQ ID NO: 46 - protein, 8 aa, Homo sapiens
IWFDGSNE

SEQ ID NO: 47 - DNA, 51 bp, Homo sapiens
gtgagagagggaatcctaggaactactaatccttatgatgcttttgatgtc

SEQ ID NO: 48 - protein, 17 aa, Homo sapiens
VREGILGTTNPYDAFDV

SEQ ID NO: 49 - DNA, 378 bp, Homo sapiens
caggtgcagctggtggagtctgggggaggcgtggtccagcctgggaggtccctgagactctcctgtgcagcgtctggattcacctttagtaactttggaatgcactgggtccgccaggcgccaggcaagggactggagtgggtggcaggtatatggtttgatggaagtaataaaaactatatagactccgtgaagggccgattcaccatctcaagagacaattccaagaacacgctgtatctgcaaatgaacagcctgagagccgaggacacggctgtgtattactgtgcgagagagggctatgattcggggactgattatatcccctatgatatttttgatatttggggccaagggacaatggtcaccgtctcttca

SEQ ID NO: 50 - protein, 126 aa, Homo sapiens
QVQLVESGGGVVQPGRSLRLSCAASGFTFSNFGMHWVRQAPGKGLEWVAGIWFDGSNKNYIDSVKGRFTISRDNSKNTLYLQMNSLRAEDTAVYYCAREGYDSGTDYIPYDIFDIWGQGTMVTVSS

SEQ ID NO: 51 - DNA, 24 bp, Homo sapiens
ggattcacctttagtaactttgga

SEQ ID NO: 52 - protein, 8 aa, Homo sapiens
GFTFSNFG

SEQ ID NO: 53 - DNA, 24 bp, Homo sapiens
atatggtttgatggaagtaataaa

SEQ ID NO: 54 - protein, 8 aa, Homo sapiens
IWFDGSNK

SEQ ID NO: 55 - DNA, 57 bp, Homo sapiens
gcgagagagggctatgattcggggactgattatatcccctatgatatttttgatatt

SEQ ID NO: 56 - protein, 19 aa, Homo sapiens
AREGYDSGTDYIPYDIFDI

SEQ ID NO: 57 - DNA, 363 bp, Homo sapiens
gaagtgcagctggtggagtctgggggaggcttggtacagcctggcacgtccctgagactctcctgtgcagcctctggattcacctttgatgattatgccatgcactgggtccggcaagctccagggaagggcctggagtgggtctcaggtattacttggaatagttataacatagactatgctgactctgtgaagggccgattcaccatctccagagacaacgccaagaactccctgtatctgcaaatgaacagtctgagagctgaggacacggccttgtattactgtgcaaaagatgatgactacagtaactacgtttactttgactactggggccagggaaccctggtcaccgtctcctca

SEQ ID NO: 58 - protein, 121 aa, Homo sapiens
EVQLVESGGGLVQPGTSLRLSCAASGFTFDDYAMHWVRQAPGKGLEWVSGITWNSYNIDYADSVKGRFTISRDNAKNSLYLQMNSLRAEDTALYYCAKDDDYSNYVYFDYWGQGTLVTVSS

SEQ ID NO: 59 - DNA, 24 bp, Homo sapiens
ggattcacctttgatgattatgcc

SEQ ID NO: 60 - protein, 8 aa, Homo sapiens
GFTFDDYA

SEQ ID NO: 61 - DNA, 24 bp, Homo sapiens
attacttggaatagttataacata

SEQ ID NO: 62 - protein, 8 aa, Homo sapiens
ITWNSYNI

SEQ ID NO: 63 - DNA, 42 bp, Homo sapiens
gcaaaagatgatgactacagtaactacgtttactttgactac

SEQ ID NO: 64 - protein, 14 aa, Homo sapiens
AKDDDYSNYVYFDY

SEQ ID NO: 65 - DNA, 360 bp, Homo sapiens
caggttcagctggtgcagtccggaactgaggtgaaggagcctggggcctcagtgaaggtctcctgtaaggcctctggttactcctttaccacctatggtatcagctggctgcgacaggcccctggacaaggacttgagtggatgggatggatcagcacttacaatggtgacacaatctctgcacagatgctccaggacagagtcaccctgaccgcagacacatccacgcgcacagcctacatggaactgagaagcctgagatctgacgacacggccgtgtattactgtgcgagaggtcatgagtatgatagtcttgtttattcttactggggccagggaaccctggtcaccgtctcctca

SEQ ID NO: 66 - protein, 120 aa, Homo sapiens
QVQLVQSGTEVKEPGASVKVSCKASGYSFTTYGISWLRQAPGQGLEWMGWISTYNGDTISAQMLQDRVTLTADTSTRTAYMELRSLRSDDTAVYYCARGHEYDSLVYSYWGQGTLVTVSS

SEQ ID NO: 67 - DNA, 24 bp, Homo sapiens
ggttactcctttaccacctatggt

SEQ ID NO: 68 - protein, 8 aa, Homo sapiens
GYSFTTYG

SEQ ID NO: 69 - DNA, 24 bp, Homo sapiens
atcagcacttacaatggtgacaca

SEQ ID NO: 70 - protein, 8 aa, Homo sapiens
ISTYNGDT

SEQ ID NO: 71 - DNA, 39 bp, Homo sapiens
gcgagaggtcatgagtatgatagtcttgtttattcttac

SEQ ID NO: 72 - protein, 13 aa, Homo sapiens
ARGHEYDSLVYSY

SEQ ID NO: 73 - DNA, 360 bp, Homo sapiens
caggtgcagctggtggagtctgggggaggcgtggtccagcctgggaggtccctgagactctcctgtgcagcgtctggattcaccttcagtagttatgccatgcactgggtccgccaggctccaggcagggggctggagtgggtggcggttatatggcatgatggagatgttgaatactatgtagactccgtgaaggaccgattcaccatctccagagacaattccaagagcacgctgtatctgcaaatgaacagcctgagagccgaagatacggctttatattattgtgcgagagaggcgtgggacctactacgtccctttgactattggggccagggaaccctggtcaccgtctcctca

SEQ ID NO: 74 - protein, 120 aa, Homo sapiens
QVQLVESGGGVVQPGRSLRLSCAASGFTFSSYAMHWVRQAPGRGLEWVAVIWHDGDVEYYVDSVKDRFTISRDNSKSTLYLQMNSLRAEDTALYYCAREAWDLLRPFDYWGQGTLVTVSS

SEQ ID NO: 75 - DNA, 24 bp, Homo sapiens
ggattcaccttcagtagttatgcc

SEQ ID NO: 76 - protein, 8 aa, Homo sapiens
GFTFSSYA

SEQ ID NO: 77 - DNA, 24 bp, Homo sapiens
atatggcatgatggagatgttgaa

SEQ ID NO: 78 - protein, 8 aa, Homo sapiens
IWHDGDVE

SEQ ID NO: 79 - DNA, 39 bp, Homo sapiens
gcgagagaggcgtgggacctactacgtccctttgactat

SEQ ID NO: 80 - protein, 13 aa, Homo sapiens
AREAWDLLRPFDY

SEQ ID NO: 81 - DNA, 351 bp, Homo sapiens
gaggtgcagctggtggagtctggaggaggcttggtccagcctggggggtccctgagactctcctgtgcagcctctgggttcatcgtcaccaccaactacatgacctggctccgccaggctccagggaaggggctggagtgggtctcacttatttatagcagtggtcacacatactacgcagactccgtgaagggccgattcaccatctccagacacaattccaagaacacactgtatctacaaatggacagcctgagagctgaggacacggccgtgtattactgtgcgagtgctttcgcagcggatgtttttgatatctggggccaagggacaatggtcaccgtctcttca

SEQ ID NO: 82 - protein, 117 aa, Homo sapiens
EVQLVESGGGLVQPGGSLRLSCAASGFIVTTNYMTWLRQAPGKGLEWVSLIYSSGHTYYADSVKGRFTISRHNSKNTLYLQMDSLRAEDTAVYYCASAFAADVFDIWGQGTMVTVSS

SEQ ID NO: 83 - DNA, 24 bp, Homo sapiens
gggttcatcgtcaccaccaactac

SEQ ID NO: 84 - protein, 8 aa, Homo sapiens
GFIVTTNY

SEQ ID NO: 85 - DNA, 21 bp, Homo sapiens
atttatagcagtggtcacaca

SEQ ID NO: 86 - protein, 7 aa, Homo sapiens
IYSSGHT

SEQ ID NO: 87 - DNA, 33 bp, Homo sapiens
gcgagtgctttcgcagcggatgtttttgatatc

SEQ ID NO: 88 - protein, 11 aa, Homo sapiens
ASAFAADVFDI

SEQ ID NO: 89 - DNA, 372 bp, Homo sapiens
caggtgcagctggtggagtctgggggaggcgtggtccagcctgggaggtccctgagactctcctgttcagcgtctggattctccttcagtcactttggcatgcactgggtccgccaggttccaggcgggggcctggagtgggtgacaagtatatggtttgatggaagtaatagatattatgcagactccttgaagggccgattcaccatctccagagacaattccaagaatactctgtatctggaaatgaacagcctgagagccgaggacacggctgtgtattactgtgtgagagaggggatactgggaactactaatccttatgatgtttttgatgtctggggtcaggggacaatggtcaccgtctcttca

SEQ ID NO: 90 - protein, 124 aa, Homo sapiens
QVQLVESGGGVVQPGRSLRLSCSASGFSFSHFGMHWVRQVPGGGLEWVTSIWFDGSNRYYADSLKGRFTISRDNSKNTLYLEMNSLRAEDTAVYYCVREGILGTTNPYDVFDVWGQGTMVTVSS

SEQ ID NO: 91 - DNA, 24 bp, Homo sapiens
ggattctccttcagtcactttggc

SEQ ID NO: 92 - protein, 8 aa, Homo sapiens
GFSFSHFG

SEQ ID NO: 93 - DNA, 24 bp, Homo sapiens
atatggtttgatggaagtaataga

SEQ ID NO: 94 - protein, 8 aa, Homo sapiens
IWFDGSNR

SEQ ID NO: 95 - DNA, 51 bp, Homo sapiens
gtgagagaggggatactgggaactactaatccttatgatgtttttgatgtc

SEQ ID NO: 96 - protein, 17 aa, Homo sapiens
VREGILGTTNPYDVFDV

SEQ ID NO: 97 - DNA, 372 bp, Homo sapiens
gaggtgcagctggtggagtctgggggaggcttggtacagccgggggggtccctgagactctcctgtgcagcctctggattcacctttagaagctatgtcatgagctggttccgccaggctccagggaaggggctggagtgggtctcaggaatgagtgggagtggtggaagcacatcctacgcagactccgtgaagggccggttcaccatctccagagacaattcaaagaatacgctgtatctgctaatgaacagcctgagaaccgaggacacggccgtatattattgtgcgaaagaaaacggggctaactggaactacggctactacggtatggacgtctggggccaagggaccacggtcaccgtctcctca

SEQ ID NO: 98 - protein, 124 aa, Homo sapiens
EVQLVESGGGLVQPGGSLRLSCAASGFTFRSYVMSWFRQAPGKGLEWVSGMSGSGGSTSYADSVKGRFTISRDNSKNTLYLLMNSLRTEDTAVYYCAKENGANWNYGYYGMDVWGQGTTVTVSS

SEQ ID NO: 99 - DNA, 24 bp, Homo sapiens
ggattcacctttagaagctatgtc

SEQ ID NO: 100 - protein, 8 aa, Homo sapiens
GFTFRSYV

SEQ ID NO: 101 - DNA, 24 bp, Homo sapiens
atgagtgggagtggtggaagcaca

SEQ ID NO: 102 - protein, 8 aa, Homo sapiens
MSGSGGST

SEQ ID NO: 103 - DNA, 51 bp, Homo sapiens
gcgaaagaaaacggggctaactggaactacggctactacggtatggacgtc

SEQ ID NO: 104 - protein, 17 aa, Homo sapiens
AKENGANWNYGYYGMDV

SEQ ID NO: 105 - DNA, 360 bp, Homo sapiens
caggtgcagctggtggagtctgggggaggcgtggtccagcctgggaggtccctgagactctcctgtgtagcgtctggattctccttcagtaactttggcatgcactgggtccgccaggctccaggcaaggggctggagtgggtggcaattatatggtatgatggaagtaataaatactattcagactccgtgaagggccgcttcaccatctccagagacaattccaagaacacgctgtatctgcaaatgaacagcctgagagtcgacgacacggctgtgtattactgtgcgagattcgatcgctggaaatttgacgcttttgatatctggggccaagggacaatggtcaccgtctcttca

SEQ ID NO: 106 - protein, 120 aa, Homo sapiens
QVQLVESGGGVVQPGRSLRLSCVASGFSFSNFGMHWVRQAPGKGLEWVAIIWYDGSNKYYSDSVKGRFTISRDNSKNTLYLQMNSLRVDDTAVYYCARFDRWKFDAFDIWGQGTMVTVSS

SEQ ID NO: 107 - DNA, 24 bp, Homo sapiens
ggattctccttcagtaactttggc

SEQ ID NO: 108 - protein, 8 aa, Homo sapiens
GFSFSNFG

SEQ ID NO: 109 - DNA, 24 bp, Homo sapiens
atatggtatgatggaagtaataaa

SEQ ID NO: 110 - protein, 8 aa, Homo sapiens
IWYDGSNK

SEQ ID NO: 111 - DNA, 39 bp, Homo sapiens
gcgagattcgatcgctggaaatttgacgcttttgatatc

SEQ ID NO: 112 - protein, 13 aa, Homo sapiens
ARFDRWKFDAFDI

SEQ ID NO: 113 - DNA, 360 bp, Homo sapiens
caggtgcagctggtggagtctgggggaggcgtggtccagcctgggaggtccctgagactctcctgtgcagcgtctggattcaccttcagtagctttgccatgcactgggtccgccaggctccaggcaaggggctggagtgggtggcagttatatggtatgatggaagtaatgattactatgcagcctccgtgaagggccgtttcaccatctccagagacaattccaagaacacgctgtatctggaaatgaacagactgagagccgaggacacggctgtgtatcactgtgcgagagataactggaattactgggggggtatggacgtctggggccaagggaccacggtcaccgtctcctca

SEQ ID NO: 114 - protein, 120 aa, Homo sapiens
QVQLVESGGGVVQPGRSLRLSCAASGFTFSSFAMHWVRQAPGKGLEWVAVIWYDGSNDYYAASVKGRFTISRDNSKNTLYLEMNRLRAEDTAVYHCARDNWNYWGGMDVWGQGTTVTVSS

SEQ ID NO: 115 - DNA, 24 bp, Homo sapiens
ggattcaccttcagtagctttgcc

SEQ ID NO: 116 - protein, 8 aa, Homo sapiens
GFTFSSFA

SEQ ID NO: 117 - DNA, 24 bp, Homo sapiens
atatggtatgatggaagtaatgat

SEQ ID NO: 118 - protein, 8 aa, Homo sapiens
IWYDGSND

SEQ ID NO: 119 - DNA, 39 bp, Homo sapiens
gcgagagataactggaattactgggggggtatggacgtc

SEQ ID NO: 120 - protein, 13 aa, Homo sapiens
ARDNWNYWGGMDV

SEQ ID NO: 121 - DNA, 375 bp, Homo sapiens
gaggtgcagctgttggagtctgggggaggctgggtgcagccgggggggtccctgagactctcctgtgcagcctctggattcgcctttagtaattatgccatgaactgggtccgccagactccagggaaggggctggagtgggtctcagttattagtagtagtggtggaaacacatactacgcagactccgtgaagggccggttcgccatctccagagacaattccagggatacgctgcatctgcaaatgaacagactgagagtcgaggacacggccgtctattactgtgcgaaagaaatacgtccgtattacgatctttcctactattacggtatggacgtctggggccaagggaccacggtcaccgtctcctca

SEQ ID NO: 122 - protein, 125 aa, Homo sapiens
EVQLLESGGGWVQPGGSLRLSCAASGFAFSNYAMNWVRQTPGKGLEWVSVISSSGGNTYYADSVKGRFAISRDNSRDTLHLQMNRLRVEDTAVYYCAKEIRPYYDLSYYYGMDVWGQGTTVTVSS

SEQ ID NO: 123 - DNA, 24 bp, Homo sapiens
ggattcgcctttagtaattatgcc

SEQ ID NO: 124 - protein, 8 aa, Homo sapiens
GFAFSNYA

SEQ ID NO: 125 - DNA, 24 bp, Homo sapiens
attagtagtagtggtggaaacaca

SEQ ID NO: 126 - protein, 8 aa, Homo sapiens
ISSSGGNT

SEQ ID NO: 127 - DNA, 54 bp, Homo sapiens
gcgaaagaaatacgtccgtattacgatctttcctactattacggtatggacgtc

SEQ ID NO: 128 - protein, 18 aa, Homo sapiens
AKEIRPYYDLSYYYGMDV

SEQ ID NO: 129 - DNA, 348 bp, Homo sapiens
caggtgcagctgcaggagtcgggcccaggactggtgaagtcttcggagaccctgtccctcacctgcactgtctctggtggctccatcagaaatttctactggagctggctccggcagcccccagggaagggactagagtggattgggcacatcaattacaatgggggcaccgactacaacccctccctcaagagtcgagtcaccatatcagtagacacgtccaagaatcagttctccctgaatttgaactctgtgaccgccgcagacacggccgtgtattactgtgcgagacagagattctacggtatggacgtctggggtccagggaccacggtcaccgtctcctca

SEQ ID NO: 130 - protein, 116 aa, Homo sapiens
QVQLQESGPGLVKSSETLSLTCTVSGGSIRNFYWSWLRQPPGKGLEWIGHINYNGGTDYNPSLKSRVTISVDTSKNQFSLNLNSVTAADTAVYYCARQRFYGMDVWGPGTTVTVSS

SEQ ID NO: 131 - DNA, 24 bp, Homo sapiens
ggtggctccatcagaaatttctac

SEQ ID NO: 132 - protein, 8 aa, Homo sapiens
GGSIRNFY

SEQ ID NO: 133 - DNA, 21 bp, Homo sapiens
atcaattacaatgggggcacc

SEQ ID NO: 134 - protein, 7 aa, Homo sapiens
INYNGGT

SEQ ID NO: 135 - DNA, 30 bp, Homo sapiens
gcgagacagagattctacggtatggacgtc

SEQ ID NO: 136 - protein, 10 aa, Homo sapiens
ARQRFYGMDV

SEQ ID NO: 137 - DNA, 324 bp, Homo sapiens
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 138 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 139 - DNA, 18 bp, Homo sapiens
cagagcattagcagctat

SEQ ID NO: 140 - protein, 6 aa, Homo sapiens
QSISSY

SEQ ID NO: 141 - DNA, 9 bp, Homo sapiens
gctgcatcc

SEQ ID NO: 142 - protein, 3 aa, Homo sapiens
AAS

SEQ ID NO: 143 - DNA, 30 bp, Homo sapiens
caacagagttacagtacccctccgatcacc

SEQ ID NO: 144 - protein, 10 aa, Homo sapiens
QQSYSTPPIT

SEQ ID NO: 145 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 146 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 147 - protein, 934 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEVGFLHSSHDVNKEASVIMLFSGLK

SEQ ID NO: 148 - protein, 281 aa, Homo sapiens
KEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKR

SEQ ID NO: 149 - protein, 1101 aa, Homo sapiens
STKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 150 - protein, 1082 aa, Homo sapiens
TKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 151 - protein, 626 aa, Homo sapiens
TKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEVGFLHSSHDVNKEASVIMLFSGLK

SEQ ID NO: 152 - protein, 650 aa, Artificial
  synthetic
TKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAEQKLISEEDLEQKLISEEDLHHHHHH

SEQ ID NO: 153 - protein, 838 aa, Artificial
  synthetic
TKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAMEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKE

SEQ ID NO: 154 - protein, 1100 aa, Artificial
  synthetic
STKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRAEFTTALQRVDLFMGQFSEVLLTSISTFVKGDLTIANLGTSEGRFMQVVVSRSGPSTPHVNFLLDSHPVSPEVIVEHPLNQNGYTLVVTGKKITKIPLNGLGCRHFQSCSQCLSAPPFVQCGWCHDKCVRSEECPSGTWTQQICLPAIYKVFPTSAPLEGGTRLTICGWDFGFRRNNKFDLKKTRVLLGNESCTLTLSESTMNTLKCTVGPAMNKHFNMSIIISNGHGTTQYSTFSYVDPIITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLHSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISIALLLLLGLFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTEQKLISEEDLEQKLISEEDLHHHHHH

SEQ ID NO: 155 - protein, 936 aa, Homo sapiens
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTEQKLISEEDLGGEQKLISEEDLHHHHHH

SEQ ID NO: 156 - protein, 13 aa, Homo sapiens
VRRLKETKDGFMF

SEQ ID NO: 157 - protein, 11 aa, Homo sapiens
LARQIGASLND

SEQ ID NO: 158 - protein, 35 aa, Homo sapiens
FIKGDLTIANLGTSEGRFMQVVVSRSGPSTPHVNF

(19) United States
(12) Patent Application Publication
(10) Pub. No.: US 2022/0040319 A1
(43) Pub. Date: Feb. 10, 2022

(54) METHODS OF TREATING OCULAR CANCER USING ANTI-MET ANTIBODIES AND BISPECIFIC ANTIGEN BINDING MOLECULES THAT BIND MET

(71) Applicant: Regeneron Pharmaceuticals, Inc., Tarrytown, NY (US)

(72) Inventors: Gary Schwartz, Briarcliff, NY (US); Oliver Surriga, New York, NY (US)

(21) Appl. No.: 16/796,380

(22) Filed: Feb. 20, 2020

Related U.S. Application Data
(60) Provisional application No. 62/808,839, filed on Feb. 21, 2019.
(60) Provisional application No. 62/823,788, filed on Mar. 26, 2019.

(51) Int. Cl. A61K 47/68; C07K 16/28; A61K 45/06; A61P 35/00

(52) U.S. Cl. CPC A61K 47/6803; A61K 47/6849; C07K 16/2863; A61K 45/06; A61K 2039/505; C07K 2317/31; C07K 2317/92; C07K 2317/33; A61P 35/00

(57) ABSTRACT

Provided herein are methods of treating ocular cancer such as uveal melanoma, orbital lymphoma, retinoblastoma, and medulloepithelioma using antibodies and bispecific antigen-binding molecules that bind MET or antibody-drug conjugates (ADCs) comprising the antibodies or bispecific antigen-binding molecules. The bispecific antigen-binding molecules comprise a first and a second antigen-binding domain, wherein the first and second antigen-binding domains bind to two different epitopes of the extracellular domain of human MET. The ADCs comprise the antibodies or bispecific antigen-binding molecules provided herein linked to a cytotoxic agent, radionuclide, or other moiety. The antibodies and bispecific antigen-binding molecules are capable of blocking the interaction between human MET and its ligand HGF. A subject having ocular cancer, for example, an uveal melanoma expressing c-Met, can be treated by administering to the subject an antibody, a bispecific antigen-binding molecule, or an ADC thereof.

### CROSS-REFERENCE TO RELATED APPLICATIONS

This application claims the benefit under 35 U.S.C § 119(e) of U.S. Provisional Application No. 62/808,839, filed Feb. 21, 2019, and U.S. Provisional Application No. 62/823,788, filed Mar. 26, 2019, which applications are herein specifically incorporated by reference in their entirety.

### TECHNICAL FIELD

The present invention relates to use of antibodies, bispecific antibodies, and antigen-binding fragments thereof, as well as antibody-drug conjugates of such antibodies, which specifically bind the hepatocyte growth factor receptor (c-Met or MET) and modulate MET signal transduction, to treat ocular cancer including uveal melanoma.

### SEQUENCE LISTING

An official copy of the sequence listing is submitted concurrently with the specification electronically via EFS-Web as an ASCII formatted sequence listing with a file name of 10548US01_SEQ_LIST_ST25.TXT, a creation date of Feb. 20, 2020, and a size of about 136 kilobytes. The sequence listing contained in this ASCII formatted document is part of the specification and is herein incorporated by reference in its entirety.

### BACKGROUND

Uveal melanoma is the most common primary intraocular malignant tumor in adults, representing 79-81% of ocular melanomas. Incidence rates in the United States are estimated at 5/million population while incidence rates in Europe range from 2 to 8/million population, depending on the latitude with decreasing incidence from north to south. Uveal melanoma has a high tendency to metastasize, resulting in poor long-term prognosis with death occurring in more than 50% cases.

Hepatocyte growth factor (HGF) (a.k.a. scatter factor [SF]) is a heterodimeric paracrine growth factor that exerts its activity by interacting with the HGF receptor (HGFR). HGFR is the product of the c-Met oncogene and is also known as MET. MET is a receptor tyrosine kinase consisting of a transmembrane beta chain linked via a disulfide bridge to an extracellular alpha chain. The binding of HGF to MET activates the kinase catalytic activity of MET resulting in the phosphorylation of Tyr 1234 and Tyr 1235 of the beta chain and subsequent activation of downstream signaling pathways.

Tumor cell lines having MET gene amplification are highly dependent on MET for growth and survival. Various monovalent MET blocking antibodies are in clinical development for the treatment of various cancers (see U.S. Pat. Nos. 5,686,292; 5,646,036; 6,099,841; 7,476,724; 9,260,531; and 9,328,173; and U.S. Patent Application Publications No. 2014/0349310 and 2005/0233960). Those antibodies include onartuzumab (MetMab) and emibetuzumab, (Xiang et al., Clin. Cancer Res. 19(18): 5068-78, 2013, and Rosen et al., Clin. Cancer Res., Published Oct. 10, 2016, doi: 10.1158/1078-0432.CCR-16-1418). Some of these antibodies block ligand-dependent MET signaling, but are not as effective in blocking ligand-independent MET activation.

Uveal melanoma tumors are characterized by mutations in G-proteins (GNAQ and GNA11) and high expression of c-Met. Targeting c-Met in uveal melanoma results in inhibition of cell invasion and metastasis, however, it does not suppress tumor growth. The rate of local tumor control and globe salvage has improved over time, but survival rate remains relatively unchanged. Antibody-drug conjugates (ADC) have advanced in the past years, several of which are approved for use by the FDA but none have been developed for uveal melanoma thus far. There remains a significant unmet medical need for improved anti-cancer drugs for use in treating eye cancer, including uveal melanoma, that potently block both ligand-dependent and ligand-independent MET signaling.

### BRIEF SUMMARY

Provided herein are methods of treating ocular cancer such as uveal melanoma orbital lymphoma, retinoblastoma, and medulloepithelioma. The methods include treatment with antibodies, antigen-binding fragments of antibodies, combinations of bivalent monospecific antibodies, or bispecific antibodies that bind human c-Met receptor protein (MET×MET). The anti-MET antibodies, and antigen-binding portions thereof, may be used alone in unmodified form, or may be included as part of an antibody-drug conjugate (ADC) or a bispecific antibody. The antibodies and ADCs are useful, inter alia, for targeting tumor cells that express MET, and thus are useful in the methods of treating ocular cancer as disclosed herein.

Other embodiments will become apparent from a review of the ensuing detailed description.

### BRIEF DESCRIPTION OF THE FIGURES

FIG. 1 is a matrix illustrating the components of 272 exemplary MET×MET bispecific antibodies disclosed herein. Each numbered cell of the matrix identifies a unique bispecific antibody comprising a “D1” antigen binding domain and a “D2” antigen binding domain, wherein the D1 antigen binding domain comprises the immunoglobulin variable domain (HCVR/LCVR amino acid sequence pair) or CDRs from the corresponding anti-MET antibody listed along the Y-axis, and wherein the D2 antigen binding domain comprises the immunoglobulin variable domain (HCVR/LCVR amino acid sequence pair) or CDRs from the corresponding anti-MET antibody listed along the X-axis.

FIG. 2 is a schematic of a luciferase-based reporter assay used to assess antibody-induced MET pathway activation or antibody blockade of HGF-induced pathway activation in HEK293T cells containing an SRE-Luciferase reporter gene construct.

FIG. 3A and FIG. 3B are line graphs depicting relative luminosity units (RLU) representing SRE-luciferase expression as a function of antibody concentration in log moles per liter. Filled squares (▪) represent parental bivalent monospecific antibody H4H13306P2, filled pyramids (▴) represent parental bivalent monospecific antibody H4H13312P2, filled circles (●) represent a monovalent antibody, filled diamonds (♦) represent isotype control, and filled inverted pyramids (▾) represent no ligand. FIG. 3A depicts antibody alone without HGF ligand. FIG. 3B depicts antibodies plus HGF ligand.

FIG. 4A and FIG. 4B are line graphs depicting relative luminosity units (RLU) representing SRE-luciferase expression as a function of antibody concentration in log moles per liter. Filled squares (▪) represent an anti-MET monovalent antibody, filled circles (●) represent a MET×MET bispecific antibody, and filled diamonds (♦) represent parental antibody H4H13312P2. FIG. 4A depicts antibody alone without HGF ligand. FIG. 4B depicts antibodies plus HGF ligand.

FIG. 5 is a bar chart depicting the relative cell growth of MET-amplified gastric cancer SNU5 cells as a function of treatment with human bivalent monospecific anti-MET antibodies 1-18, a control antibody and an anti-MET monovalent antibody. For comparison purposes, antibody 8 (abscissa) is parental antibody H4H13306P2, and antibody 11 (abscissa) is parental antibody H4H13312P2.

FIG. 6A and FIG. 6B contain bar charts depicting the relative cell growth of MET-amplified cells as a function of treatment with a MET×MET bispecific antibody, a control antibody and an anti-MET monovalent antibody. FIG. 6A depicts the relative growth of SNU5 cells as a function of treatment with control antibody, a monovalent antibody at 0.1, 1 and 10 μg/mL, and a MET×MET bispecific antibody at 0.1, 1 and 10 μg/mL. FIG. 6B depicts the relative growth of EBC-1 cells as a function of treatment with control antibody and a MET×MET bispecific antibody at 0.1 and 1 μg/mL.

FIG. 7A and FIG. 7B. FIG. 7A depicts immunoblots of pMET (phosphorylated MET), MET, pErk (phosphorylated Erk), and tubulin (for loading control) extracted from Hs746T cells after treatment with a control antibody and a MET×MET bispecific antibody. FIG. 7B depicts the expression of MET (and tubulin as a loading control) in Hs746T cells after treatment with the MET×MET bispecific antibody for 0, 2 and 6 hours.

FIG. 8 depicts an immunoblot of pMET, MET, pErk, and tubulin (for loading control) extracted from Hs746T cells after treatment with a control antibody, a MET×MET bispecific antibody, an anti-MET monospecific bivalent parent antibody 1, an anti-MET monospecific bivalent parent antibody 2, and a combination of parental antibodies 1 and 2.

FIG. 9 depicts an immunoblot of the expression of MET (and tubulin as a loading control) in Hs746T cells after treatment with a control antibody and a MET×MET bispecific antibody for 2, 6 and 18 hours.

FIG. 10A and FIG. 10B. FIG. 10A depicts immunoblots of pMET, MET, pErk, and tubulin (for loading control) extracted from SNU5 cells after treatment with a control antibody and a MET×MET bispecific antibody. FIG. 10B depicts the expression of MET (and tubulin as a loading control) in SNU5 cells after treatment with a control antibody and an anti-MET monovalent antibody.

FIG. 11 depicts an immunoblot of pMET, MET, pErk, and tubulin (for loading control) extracted from EBC-1 cells after treatment with a control antibody and a MET×MET bispecific antibody.

FIG. 12 is a line graph depicting the change in EBC-1 tumor volume in cubic millimeters as a function of time in days after implantation of EBC-1 cells in animals treated with control antibody (filled square ▪), MET monovalent antibody (filled circle ●), or MET×MET bispecific antibody (filled diamond ♦).

FIG. 13A and FIG. 13B contain bar charts depicting the relative cell growth of MET-amplified cells as a function of treatment with a MET×MET bispecific antibody, a control antibody and an anti-MET monovalent antibody. FIG. 13A depicts the relative growth of Hs746T cells as a function of treatment with control antibody, a MET×MET bispecific antibody, the MET×MET parental monospecific antibody 1, the MET×MET parental monospecific antibody 2, and a combination of parental antibodies 1 and 2. FIG. 13B depicts the relative growth of Hs746T cells as a function of treatment with control antibody, a monovalent antibody at 1, 10 and 25 μg/mL, and a MET×MET bispecific antibody at 1, 10 and 25 μg/mL.

FIG. 14 is a bar chart depicting the relative cell growth of NCI-H596 cells as a function of treatment with a control antibody (C), a MET×MET bispecific antibody (MM), the MET×MET parental monospecific antibody 1 (M1), the MET×MET parental monospecific antibody 2 (M2), a combination of parental antibodies 1 and 2 (M1M2), and the MET-agonist hepatocyte growth factor (HGF).

FIG. 15 is a line graph depicting the change in Hs746T tumor volume in cubic millimeters as a function of time in days after implantation of Hs746T cells in animals treated with control antibody (filled square ▪), MET monovalent antibody (filled circle ●), or MET×MET bispecific antibody (filled diamond ♦).

FIG. 16A is a line graph depicting the change in SNUS tumor volume in cubic millimeters as a function of time in days after implantation of SNUS cells in animals treated with control antibody (filled square ▪), MET monovalent antibody at 1 mg/mL (filled circle ●), MET monovalent antibody at 10 mg/mL (open circle ◯), MET×MET bispecific antibody at 1 mg/mL (filled diamond ♦), or MET×MET bispecific antibody at 10 mg/mL (open diamond ⋄).

FIG. 16B is an immunoblot of pMET, MET, and tubulin (loading control) extracted from an SNUS tumor removed from a mouse xenograft model after treatment with a control antibody, 10 mg/kg of an anti-MET monovalent antibody, and 10 mg/kg of a MET×MET bispecific antibody.

FIG. 17 is a line graph depicting the change in U87-MG tumor volume in cubic millimeters as a function of time in days after implantation of U87-MG cells in animals treated with control antibody (filled square ▪), MET monovalent antibody (filled circle ●), or MET×MET bispecific antibody (filled diamond ♦).

FIG. 18 is a line graph depicting the change in U118-MG tumor volume in cubic millimeters as a function of time in days after implantation of U118-MG cells in animals treated with control antibody (filled square ▪), MET monovalent antibody (filled circle ●), or MET×MET bispecific antibody (open diamond ⋄).

FIG. 19 is a schematic illustrating the synthesis of maytansinoid 6.

FIG. 20 is a schematic illustrating the synthesis of maytansinoid intermediate 1.

FIG. 21A, FIG. 21B, FIG. 21C, and FIG. 21D are line graphs depicting the change in cell viability in four c-Met expressing uveal melanoma cells treated with two different concentrations of bispecific c-Met antibody conjugated to Maytansinoid B (filled circle ●) compared to isotype antibody conjugated with Maytansinoid B (filled triangle ▴) over 7 days.

FIG. 22A and FIG. 22B are line graphs depicting the change in cell viability in c-Met expressing OMM1.3 cells versus c-Met negative OCM3 cells when treated with bispecific c-Met antibody conjugated to Maytansinoid B (0.3 to 10 nM) (cross hatched line) or isotype antibody conjugated with Maytansinoid B (filled square ▪) over 7 days.

FIG. 23 and FIG. 24 are bar graphs depicting percent apoptosis resulting from treatment of uveal melanoma cells treated with two different concentrations (1.25 nM, FIG. 23; 2.5 nM, FIG. 24) of bispecific c-Met antibody conjugated to Maytansinoid B compared to isotype antibody conjugated with Maytansinoid B.

FIG. 25, FIG. 26, and FIG. 27 are histograms (with inset side scatter plots) depicting cellular distribution in each of the growth phases after treatment with bispecific c-Met antibody conjugated to Maytansinoid B compared to isotype antibody conjugated with Maytansinoid B. Two c-Met positive cells lines were tested, OMM1.3 (FIG. 25) and Mel202 (FIG. 26), and compared to a c-Met negative cell line, OCM3 (FIG. 27).

FIG. 28 is an image of a Western blot showing c-Met expression levels of several uveal melanoma cell lines as well as SNU-5, a positive control gastric carcinoma cell line known to highly express c-Met, and A549, a lung carcinoma cell line that also express c-Met.

FIG. 29 is an image of a Western blot demonstrating PARP cleavage and histone H3 phosphorylation in three uveal melanoma cell lines after 24 hours of treatment with a bispecific c-Met antibody conjugated to Maytansinoid B compared to an isotype antibody conjugated with Maytansinoid B.

FIG. 30 is an image of a Western blot showing time-dependent induction of PARP cleavage, c-Met protein expression, and histone H3 phosphorylation in a c-Met positive cell line, OMM1.3, compared to a c-Met negative cell line, OCM3, after treatment with a bispecific c-Met antibody conjugated to Maytansinoid B compared to an isotype antibody conjugated with Maytansinoid B.

FIG. 31 illustrates an ‘H-NMR spectrum of Maytansin-3-N-methyl-L-alanine-propanamidyl-3-thio-3-succinimidyl-N-methylcyclohexyl-4-trans-carboxysuccinate. The spectrum is not complicated by resonances attributable to a mixture and is consistent with a single diastereomer present in at least 95% diastereomeric excess.

FIG. 32 provides images demonstrating inhibition of cell invasion in OMM1.3 cells treated with increasing doses of control antibody, control-ADC, MET×MET and MET×MET-ADC while using 50 ng/ml HGF as chemoattractant. The MET×MET antibody and MET×MET-ADC potently inhibited cell invasion at picomolar doses in which cell viability is not affected.

FIG. 33 illustrates the dose-dependent decrease in cell viability of uveal melanoma cells treated with a bispecific c-Met antibody conjugated to Maytansinoid B compared to isotype antibody conjugated with Maytansinoid B.

### DETAILED DESCRIPTION

Before the present invention is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this technology belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### MET Protein

The expressions “MET,” “c-Met,” and the like, as used herein, refer to the human membrane spanning receptor tyrosine kinase comprising (1) the amino acid sequence as set forth in SEQ ID NO:145, and/or having the amino acid sequence as set forth in NCBI accession No. NM_001127500.2, representing the unprocessed preproprotein of isoform “a”, (2) the amino acid sequence as set forth in SEQ ID NO:146, and/or having the amino acid sequence as set forth in NCBI accession No. NM_000236.2, representing the unprocessed preproprotein of isoform “b”, (3) the amino acid sequence as set forth in SEQ ID NO:147, and/or having the amino acid sequence as set forth in NCBI accession No. NM_001311330.1, representing the unprocessed preproprotein of isoform “c”, and/or (3) the mature protein comprising the cytoplasmic alpha subunit (SEQ ID NO:148) shared by all three isoforms and the transmembrane beta subunit (SEQ ID NO:149, 150, or 151 of isoform a, b and c, respectively). The expression “MET” includes both monomeric and multimeric MET molecules. As used herein, the expression “monomeric human MET” means a MET protein or portion thereof that does not contain or possess any multimerizing domains and that exists under normal conditions as a single MET molecule without a direct physical connection to another MET molecule. An exemplary monomeric MET molecule is the molecule referred to herein as “hMET.mmh” comprising the amino acid sequence of SEQ ID NO:152 (see, e.g., Example 3, herein). As used herein, the expression “dimeric human MET” means a construct comprising two MET molecules connected to one another through a linker, covalent bond, non-covalent bond, or through a multimerizing domain such as an antibody Fc domain. An exemplary dimeric MET molecule is the molecule referred to herein as “hMET.mFc” comprising the amino acid sequence of SEQ ID NO:153 (see, e.g., Example 3, herein).

All references to proteins, polypeptides and protein fragments herein are intended to refer to the human version of the respective protein, polypeptide or protein fragment unless explicitly specified as being from a non-human species. Thus, the expression “MET” means human MET unless specified as being from a non-human species, e.g., “mouse MET,” “monkey MET,” etc.

As used herein, the expression “cell surface-expressed MET” means one or more MET protein(s), or the extracellular domain thereof, that is/are expressed on the surface of a cell in vitro or in vivo, such that at least a portion of a MET protein is exposed to the extracellular side of the cell membrane and is accessible to an antigen-binding portion of an antibody. A “cell surface-expressed MET” can comprise or consist of a MET protein expressed on the surface of a cell which normally expresses MET protein. Alternatively, “cell surface-expressed MET” can comprise or consist of MET protein expressed on the surface of a cell that normally does not express human MET on its surface but has been artificially engineered to express MET on its surface.

### Therapeutic Methods of Treating Ocular Cancer

Provided herein are methods of treating ocular cancer such as, for example, uveal melanoma, orbital lymphoma, retinoblastoma, and medulloepithelioma. In some aspects, the method comprises administering to a subject in need thereof a therapeutic composition comprising an anti-MET antibody or a MET×MET bispecific antigen-binding molecule (e.g., an anti-MET comprising any of the HCVR/LCVR or CDR sequences as set forth in Table 1 herein, or a MET×MET bispecific antigen-binding molecule comprising any of the D1 and D2 components as set forth in Table 5 herein, or an anti-MET antibody selected from the group consisting of onartuzumab, emibetuzumab, telisotuzumab, SAIT301, ARGX-111, Sym015, HuMax-cMet, and CE-355621). In some embodiments, the anti-MET antibody or a MET×MET bispecific antigen-binding molecule is conjugated to a cytotoxic compound such as a maytansinoid, as described in detail below. The therapeutic composition can comprise any of the anti-MET antibodies or MET×MET bispecific antigen-binding molecules disclosed herein, including anti-MET ADCs or MET×MET bispecific antigen-binding molecule conjugated to a cytotoxic agent, and a pharmaceutically acceptable carrier or diluent.

Uveal melanoma is the most common malignant primary intraocular tumor in adults. These tumors can occur in the choroid, iris and ciliary body, and are sometimes called iris or ciliary body melanomas. Uveal melanoma is highly metastatic. Other ocular cancers include orbital lymphoma, retinoblastoma, and medulloepithelioma, the latter of which can occur in the ciliary body and uvea. It is contemplated that the methods disclosed herein are useful in treating ocular cancers such as orbital lymphoma, retinoblastoma, and medulloepithelioma. In some aspects, treating includes inhibiting or mitigating invasion and/or metastasis from the primary tumor.

The anti-MET antibodies and MET×MET bispecific antigen-binding molecules, and drug conjugates thereof, are useful, inter alia, for the treatment, prevention and/or amelioration of any disease or disorder associated with or mediated by MET expression, signaling or activity, or treatable by blocking the interaction between MET and HGF, or otherwise inhibiting MET activity and/or signaling, and/or promoting receptor internalization and/or decreasing cell surface receptor number. In particular, the anti-MET antibodies and MET×MET bispecific antigen-binding molecules, and drug conjugates thereof, are useful in treating uveal melanoma. Treatment includes reducing uveal melanoma tumor growth and/or causing regression of an uveal melanoma in a subject. Treatment also includes inhibiting or mitigating invasion of uveal melanoma cells, or inhibiting or mitigating metastasis of uveal melanoma from the primary tumor.

For example, anti-MET antibodies and MET×MET bispecific antigen-binding molecules of the present disclosure are useful for the treatment of uveal melanoma tumors that express (or overexpress) MET. For example, the anti-MET antibodies and MET×MET bispecific antigen-binding molecules may be used to treat primary and/or metastatic tumors arising in the eye.

As such, provided herein is a method of treating eye cancer, reducing growth of an eye cancer, inhibiting or mitigating invasion and/or metastasis, and/or causing regression of an eye cancer in a subject. For example, provided herein is a method of treating an uveal melanoma, reducing uveal melanoma tumor growth, inhibiting or mitigating invasion and/or metastasis, and/or causing regression of an uveal melanoma in a subject. In some aspects, the eye cancer, for example, the uveal melanoma, expresses MET. In some aspects, the method comprises administering to a subject in need thereof an antibody-drug conjugate (ADC) comprising a bispecific antigen-binding molecule and a cytotoxin, wherein the bispecific antigen-binding molecule comprises: a first antigen-binding domain (D1); and a second antigen-binding domain (D2); wherein D1 specifically binds a first epitope of human MET; and wherein D2 specifically binds a second epitope of human MET.

Further provided herein is a method of inhibiting proliferation, inhibiting invasion, causing apoptosis, and/or decreasing viability of an uveal melanoma cell. In some embodiments, the method comprises contacting the cell with an antibody-drug conjugate (ADC) comprising a bispecific antigen-binding molecule and a cytotoxin. In some embodiments, the bispecific antigen-binding molecule comprises: a first antigen-binding domain (D1); and a second antigen-binding domain (D2); wherein D1 specifically binds a first epitope of human MET; and wherein D2 specifically binds a second epitope of human MET.

Still further provided herein is a method of inducing mitotic arrest of an uveal melanoma cell. In some embodiments, the method comprises contacting the cell with an antibody-drug conjugate (ADC) comprising a bispecific antigen-binding molecule and a cytotoxin, wherein the bispecific antigen-binding molecule comprises: a first antigen-binding domain (D1); and a second antigen-binding domain (D2); wherein D1 specifically binds a first epitope of human MET; and wherein D2 specifically binds a second epitope of human MET.

Also provided herein is a method of treating eye cancer in a subject suffering from a c-Met expressing tumor. In some embodiments, the method comprises administering to the subject a bispecific antigen-binding molecule comprising: a first antigen-binding domain (D1); and a second antigen-binding domain (D2); wherein D1 specifically binds a first epitope of human MET; and wherein D2 specifically binds a second epitope of human MET. In some aspects, the bispecific antigen-binding molecule is conjugated to a cytotoxin to form an antibody-drug conjugate (ADC). In some aspects, the cytotoxin is a maytansinoid.

Various aspects of the bispecific antigen-binding molecule and various aspects of the cytotoxin are provided in the following paragraphs, though described in greater detail elsewhere herein.

In some aspects, D1 and D2 do not compete with one another for binding to human MET. In some aspects, the first epitope of human MET comprises amino acids 192-204 of SEQ ID NO:155. In some aspects, the second epitope of human MET comprises amino acids 305-315 and 421-455 of SEQ ID NO:155. In some aspects, the first epitope of human MET comprises amino acids 192-204 of SEQ ID NO:155; and the second epitope of human MET comprises amino acids 305-315 and 421-455 of SEQ ID NO:155.

In some embodiments, D1 comprises three heavy chain complementarity determining regions (HCDR1, HCDR2 and HCDR3) within a heavy chain variable region (HCVR) comprising the amino acid sequence of SEQ ID NO: 58 or SEQ ID NO: 18 and three light chain complementarity determining regions (LCDR1, LCDR2 and LCDR3) within a light chain variable region (LCVR) comprising the amino acid sequence of SEQ ID NO:138. In some embodiments, D2 comprises three heavy chain complementarity determining regions (HCDR1, HCDR2 and HCDR3) within a heavy chain variable region (HCVR) comprising the amino acid sequence of SEQ ID NO: 82 and three light chain complementarity determining regions (LCDR1, LCDR2 and LCDR3) within a light chain variable region (LCVR) comprising the amino acid sequence of SEQ ID NO: 138.

In some embodiments, the bispecific antigen-binding molecule comprises the CDRs within the D1-HCVR amino acid sequence of SEQ ID NO: 58 and the CDRs within the D2-HCVR amino acid sequence of SEQ ID NO: 82. In some embodiments, the bispecific antigen-binding molecule comprises the CDRs within the D1-HCVR amino acid sequence of SEQ ID NO: 18 and the CDRs within the D2-HCVR amino acid sequence of SEQ ID NO: 82.

In some aspects, the bispecific antigen-binding molecule D1 comprises three heavy chain complementarity determining regions (HCDR1, HCDR2 and HCDR3) within a heavy chain variable region (HCVR) comprising the amino acid sequence of SEQ ID NO:58 or an amino acid sequence that is at least 95% identical thereto and three light chain complementarity determining regions (LCDR1, LCDR2 and LCDR3) within a light chain variable region (LCVR) comprising the amino acid sequence of SEQ ID NO:138 or an amino acid sequence that is at least 95% identical thereto.

In some aspects, the D1 HCDR1 comprises the amino acid sequence of SEQ ID NO:60; HCDR2 comprises the amino acid sequence of SEQ ID NO:62; HCDR3 comprises the amino acid sequence of SEQ ID NO:64; LCDR1 comprises the amino acid sequence of SEQ ID NO:140; LCDR2 comprises the amino acid sequence of SEQ ID NO:142; and LCDR3 comprises the amino acid sequence of SEQ ID NO:144.

In some aspects, the bispecific antigen-binding molecule D1 comprises an HCVR comprising the amino acid sequence of SEQ ID NO: 58 or an amino acid sequence that is at least 95% identical thereto; and an LCVR comprising the amino acid sequence of SEQ ID NO: 138 or an amino acid sequence that is at least 95% identical thereto.

In some aspects, the bispecific antigen-binding molecule D1 comprises an HCVR comprising the amino acid sequence of SEQ ID NO: 58; and an LCVR comprising the amino acid sequence of SEQ ID NO: 138.

In some aspects, the bispecific antigen-binding molecule D2 comprises three heavy chain complementarity determining regions (HCDR1, HCDR2 and HCDR3) within a heavy chain variable region (HCVR) comprising the amino acid sequence of SEQ ID NO: 82 or an amino acid sequence that is at least 95% identical thereto and three light chain complementarity determining regions (LCDR1, LCDR2 and LCDR3) within a light chain variable region (LCVR) comprising the amino acid sequence of SEQ ID NO: 138 or an amino acid sequence that is at least 95% identical thereto.

In some aspects, the bispecific antigen-binding molecule D2 HCDR1 comprises the amino acid sequence of SEQ ID NO: 84; HCDR2 comprises the amino acid sequence of SEQ ID NO: 86; HCDR3 comprises the amino acid sequence of SEQ ID NO: 88; LCDR1 comprises the amino acid sequence of SEQ ID NO:140; LCDR2 comprises the amino acid sequence of SEQ ID NO: 142; and LCDR3 comprises the amino acid sequence of SEQ ID NO: 144.

In some aspects, the bispecific antigen-binding molecule D2 comprises an HCVR comprising the amino acid sequence of SEQ ID NO: 82 or an amino acid sequence that is at least 95% identical thereto; and an LCVR comprising the amino acid sequence of SEQ ID NO: 138 or an amino acid sequence that is at least 95% identical thereto.

In some aspects, the bispecific antigen-binding molecule D2 comprises an HCVR comprising the amino acid sequence of SEQ ID NO: 82; and an LCVR comprising the amino acid sequence of SEQ ID NO: 138.

In some embodiments of the methods provided herein, the cytotoxin is selected from the group consisting of biotoxins, chemotherapeutic agents, and radioisotopes. For example, the cytotoxin can be selected from the group consisting of maytansinoids, auristatins, tomaymycins, duocarmycins, ²²⁵Ac, ²²⁷Th, and any derivatives thereof. In some aspects, the cytotoxin is conjugated to the bispecific antigen-binding molecule through a linker.

An exemplary cytotoxin is:

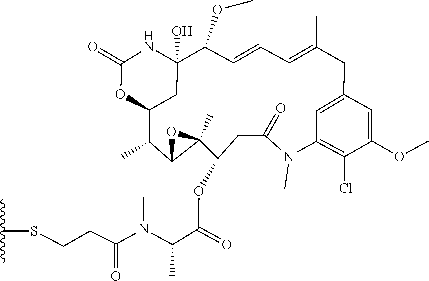 [SMILES: *SCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

wherein the [symbol] is the bond to a linker. In some aspects the linker is:

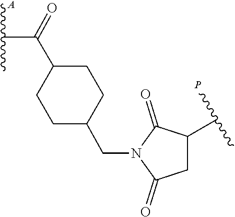

wherein the bond noted with [symbol] represents the bond to the bispecific antigen-binding molecule and the bond noted with [symbol] represents the bond to the cytotoxin.

A further exemplary cytotoxin is:

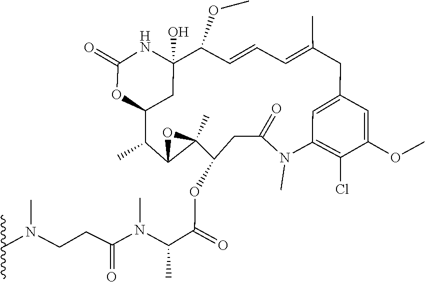 [SMILES: *N(C)CCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

wherein the [symbol] is the bond to the linker. In some aspects, the linker is

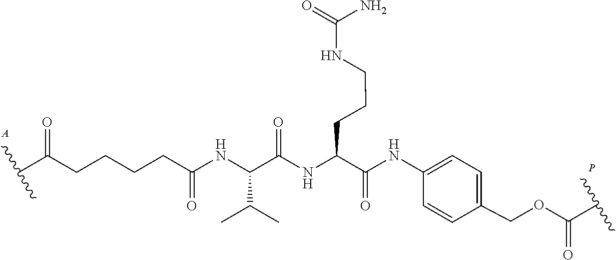

wherein the bond noted with [symbol] represents the bond to the bispecific antigen-binding molecule and the bond noted with [symbol] represents the bond to the cytotoxin.

The methods provided herein are useful in treating ocular cancer or eye cancer. In some embodiments, the eye cancer is selected from the group consisting of uveal melanoma, orbital lymphoma, retinoblastoma, and medulloepithelioma.

In the context of the methods of treatment described herein, the anti-MET antibodies and MET×MET bispecific antigen-binding molecules, and drug conjugates thereof, may be administered as a monotherapy (i.e., as the only therapeutic agent) or in combination with one or more additional therapeutic agents (examples of which are described elsewhere herein).

### Anti-MET Antibodies and Antigen-Binding Fragments Thereof

In further detail, and according to one aspect, anti-MET antibodies useful according to the methods provided herein are listed in Tables 1 and 2 herein. Table 1 sets forth the amino acid sequence identifiers of the heavy chain variable regions (HCVRs), light chain variable regions (LCVRs), heavy chain complementarity determining regions (HCDR1, HCDR2 and HCDR3), and light chain complementarity determining regions (LCDR1, LCDR2 and LCDR3) of the exemplary anti-MET antibodies from which the bispecific antigen-binding molecules (used interchangeably herein with bispecific antigen-binding protein) disclosed herein may be derived. Table 2 sets forth the nucleic acid sequence identifiers of the HCVRs, LCVRs, HCDR1, HCDR2 HCDR3, LCDR1, LCDR2 and LCDR3 of the exemplary anti-MET antibodies.

Also useful according to the methods provided herein are anti-MET antibodies selected from the group consisting of onartuzumab, emibetuzumab, telisotuzumab, SAIT301, ARGX-111, Sym015, HuMax-cMet, and CE-355621.

Also useful according to the methods provided herein are antibodies or antigen-binding fragments thereof that specifically bind MET and agonize (e.g., activate) the MET signaling pathway in cells, as well as the use of such antibodies in therapeutic settings where activation of MET signaling would be beneficial or therapeutically useful. Non-limiting examples of such an agonist anti-MET antibodies include the antibody referred to herein as “H4H14636D,” as well as antibodies and antigen-binding fragments thereof comprising the heavy and light chain CDRs (SEQ ID NOs: 28, 30, 32, 140, 142, 144) and/or heavy and light chain variable domains (SEQ ID NOs: 26/138) thereof.

Useful herein are antibodies or antigen-binding fragments thereof that specifically bind MET, comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

Useful herein are antibodies or antigen-binding fragments thereof that specifically bind MET, comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

Useful herein antibodies or antigen-binding fragments thereof that specifically bind MET, comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, antibodies, or antigen-binding fragments thereof, comprise an HCVR/LCVR amino acid sequence pair contained within any of the exemplary anti-MET antibodies listed in Table 1. In certain embodiments, the HCVR/LCVR amino acid sequence pair is selected from the group consisting of: SEQ ID NO: 2/138, 10/138, 18/138, 26/138, 34/138, 42/138, 50/138, 58/138, 66/138, 74/138, 82/138, 90/138, 98/138, 106/138, 114/138, 122/138 and 130/138.

Also useful are antibodies or antigen-binding fragments thereof that specifically bind MET, comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

Also useful are antibodies or antigen-binding fragments thereof that specifically bind MET, comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

Also useful are antibodies or antigen-binding fragments thereof that specifically bind MET, comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

Also useful are antibodies or antigen-binding fragments thereof that specifically bind MET, comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

Also useful are antibodies or antigen-binding fragments thereof that specifically bind MET, comprising an HCDR1 and an LCDR1 amino acid sequence pair (HCDR1/LCDR1) comprising any of the HCDR1 amino acid sequences listed in Table 1 paired with any of the LCDR1 amino acid sequences listed in Table 1. According to certain embodiments, antibodies, or antigen-binding fragments thereof, comprise an HCDR3/LCDR3 amino acid sequence pair contained within any of the exemplary anti-MET antibodies listed in Table 1. In certain embodiments, the HCDR1/LCDR1 amino acid sequence pair is selected from the group consisting of: SEQ ID NO: 4/140, 12/140, 20/140, 28/140, 36/140, 44/140, 52/140, 60/140, 68/140, 76/140, 84/140, 92/140, 100/140, 108/140, 116/140, 124/140 and 132/140.

Also useful are antibodies or antigen-binding fragments thereof that specifically bind MET, comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

Also useful are antibodies or antigen-binding fragments thereof that specifically bind MET, comprising an HCDR2 and an LCDR2 amino acid sequence pair (HCDR2/LCDR2) comprising any of the HCDR2 amino acid sequences listed in Table 1 paired with any of the LCDR2 amino acid sequences listed in Table 1. According to certain embodiments, antibodies, or antigen-binding fragments thereof, comprise an HCDR2/LCDR2 amino acid sequence pair contained within any of the exemplary anti-MET antibodies listed in Table 1. In certain embodiments, the HCDR2/LCDR2 amino acid sequence pair is selected from the group consisting of: SEQ ID NO: 6/142, 14/142, 22/142, 30/142, 38/142, 46/142, 54/142, 62/142, 70/142, 78/142, 86/142, 94/142, 102/142, 110/142, 118/142, 126/142, and 134/142.

Also useful are antibodies or antigen-binding fragments thereof that specifically bind MET, comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

Also useful herein are antibodies or antigen-binding fragments thereof that specifically bind MET, comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1. According to certain embodiments, antibodies, or antigen-binding fragments thereof, comprise an HCDR3/LCDR3 amino acid sequence pair contained within any of the exemplary anti-MET antibodies listed in Table 1. In certain embodiments, the HCDR3/LCDR3 amino acid sequence pair is selected from the group consisting of: SEQ ID NO: 8/144, 16/144, 24/144, 32/144, 40/144, 48/144, 56/144, 64/144, 72/144, 80/144, 88/144, 96/144, 104/144, 112/144, 120/144, 128/144 and 136/144.

Also useful herein are antibodies or antigen-binding fragments thereof that specifically bind MET, comprising a set of six CDRs (i.e., HCDR1-HCDR2-HCDR3-LCDR1-LCDR2-LCDR3) contained within any of the exemplary anti-MET antibodies listed in Table 1. In certain embodiments, the HCDR1-HCDR2-HCDR3-LCDR1-LCDR2-LCDR3 amino acid sequences set is selected from the group consisting of: SEQ ID NO: 4-6-8-140-142-144, 12-14-16-140-142-144, 20-22-24-140-142-144, 28-30-32-140-142-144, 36-38-40-140-142-144, 44-44-48-140-142-144, 52-54-56-140-142-144, 60-62-64-140-142-144, 68-70-72-140-142-144, 76-78-80-140-142-144, 84-86-88-140-142-144, 92-94-96-140-142-144, 100-102-104-140-142-144, 108-110-112-140-142-144, 116-118-120-140-142-144, 124-126-128-140-142-144 and 132-134-136-140-142-144.

In a related embodiment, antibodies, or antigen-binding fragments thereof that specifically bind MET and are useful in the methods disclosed herein, comprise a set of six CDRs (i.e., HCDR1-HCDR2-HCDR3-LCDR1-LCDR2-LCDR3) contained within an HCVR/LCVR amino acid sequence pair as defined by any of the exemplary anti-MET antibodies listed in Table 1. For example, antibodies or antigen-binding fragments thereof that specifically bind MET, comprise the HCDR1-HCDR2-HCDR3-LCDR1-LCDR2-LCDR3 amino acid sequences set contained within an HCVR/LCVR amino acid sequence pair selected from the group consisting of: SEQ ID NO: 4-6-8-140-142-144, 12-14-16-140-142-144, 20-22-24-140-142-144, 28-30-32-140-142-144, 36-38-40-140-142-144, 44-44-48-140-142-144, 52-54-56-140-142-144, 60-62-64-140-142-144, 68-70-72-140-142-144, 76-78-80-140-142-144, 84-86-88-140-142-144, 92-94-96-140-142-144, 100-102-104-140-142-144, 108-110-112-140-142-144, 116-118-120-140-142-144, 124-126-128-140-142-144 and 132-134-136-140-142-144.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

Also useful according to the methods provided herein are anti-MET antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

### MET×MET Bispecific Antigen-Binding Molecules

The present inventors have observed that certain monospecific anti-MET antigen binding molecules that block HGF binding to MET tend to potently activate MET signaling (an undesirable consequence for a therapeutic molecule). The present inventors have surprisingly discovered, however, that bispecific antigen-binding molecules that simultaneously bind to two separate epitopes on the MET protein extracellular domain are effective at blocking ligand binding to MET while causing little agonism of MET signaling. Furthermore, the present inventors have surprisingly discovered that the bispecific antigen-binding molecules are exceptionally suited for treating ocular cancer such as uveal melanoma, orbital lymphoma, retinoblastoma, and medulloepithelioma, and/or inhibiting or mitigating metastasis.

Accordingly, useful according to the methods described herein are bispecific antigen binding molecules comprising a first antigen-binding domain (also referred to herein as “D1”), and a second antigen-binding domain (also referred to herein as “D2”). The simultaneous binding of the two separate MET epitopes by the bispecific antigen-binding molecule results in effective ligand blocking with minimal activation of MET signaling.

The bispecific antigen-binding molecules, which comprise a first antigen-binding domain (D1) which specifically binds a first epitope of human MET and a second antigen-binding domain (D2) which specifically binds a second epitope of human MET, may be referred to herein as “MET×MET bispecific antibodies,” “MET×MET,” or other related terminology. In some embodiments, the first epitope of human MET comprises amino acids 192-204 of SEQ ID NO:155. In some embodiments, the second epitope of human MET comprises amino acids 305-315 and 421-455 of SEQ ID NO:155. In some embodiments, the first epitope of human MET comprises amino acids 192-204 of SEQ ID NO:155; and the second epitope of human MET comprises amino acids 305-315 and 421-455 of SEQ ID NO:155.

In certain embodiments, D1 and D2 domains of a MET×MET bispecific antibody are non-competitive with one another. Non-competition between D1 and D2 for binding to MET means that, the respective monospecific antigen binding proteins from which D1 and D2 were derived do not compete with one another for binding to human MET. Exemplary antigen-binding protein competition assays are known in the art, non-limiting examples of which are described elsewhere herein.

In certain embodiments, D1 and D2 bind to different (e.g., non-overlapping, or partially overlapping) epitopes on MET, as described elsewhere herein.

MET×MET bispecific antigen-binding molecules may be constructed using the antigen-binding domains of two separate monospecific anti-MET antibodies. For example, a collection of monoclonal monospecific anti-MET antibodies may be produced using standard methods known in the art. The individual antibodies thus produced may be tested pairwise against one another for cross-competition to a MET protein. If two different anti-MET antibodies are able to bind to MET at the same time (i.e., do not compete with one another), then the antigen-binding domain from the first anti-MET antibody and the antigen-binding domain from the second, non-competitive anti-MET antibody can be engineered into a single MET×MET bispecific antibody in accordance with the present disclosure.

According to the present disclosure, a bispecific antigen-binding molecule can be a single multifunctional polypeptide, or it can be a multimeric complex of two or more polypeptides that are covalently or non-covalently associated with one another. As will be made evident by the present disclosure, any antigen binding construct which has the ability to simultaneously bind two separate, non-identical epitopes of the MET molecule is regarded as a bispecific antigen-binding molecule. Any of the bispecific antigen-binding molecules described herein, or variants thereof, may be constructed using standard molecular biological techniques (e.g., recombinant DNA and protein expression technology) as will be known to a person of ordinary skill in the art.

### Antigen-Binding Domains

The bispecific antigen-binding molecules useful in the methods disclosed herein comprise two separate antigen-binding domains (D1 and D2). As used herein, the expression “antigen-binding domain” means any peptide, polypeptide, nucleic acid molecule, scaffold-type molecule, peptide display molecule, or polypeptide-containing construct that is capable of specifically binding a particular antigen of interest (e.g., human MET). The term “specifically binds” or the like, as used herein, means that the antigen-binding domain forms a complex with a particular antigen characterized by a dissociation constant (K_{D}) of 500 μM or less, and does not bind other unrelated antigens under ordinary test conditions. “Unrelated antigens” are proteins, peptides or polypeptides that have less than 95% amino acid identity to one another.

Exemplary categories of antigen-binding domains that can be used in the context of the present disclosure include antibodies, antigen-binding portions of antibodies, peptides that specifically interact with a particular antigen (e.g., peptibodies), receptor molecules that specifically interact with a particular antigen, proteins comprising a ligand-binding portion of a receptor that specifically binds a particular antigen, antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, 2011, Curr. Opin. Biotechnol. 22:849-857, and references cited therein]), and aptamers or portions thereof.

Methods for determining whether two molecules specifically bind one another are well known in the art and include, for example, equilibrium dialysis, surface plasmon resonance, and the like. For example, an antigen-binding domain, as used in the context of the present disclosure, includes polypeptides that bind a particular antigen (e.g., a target molecule [T] or an internalizing effector protein [E]) or a portion thereof with a K_{D} of less than about 500 μM, less than about 400 μM, less than about 300 μM, less than about 200 μM, less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 5 μM, less than about 4 μM, less than about 2 μM, less than about 1 μM, less than about 0.5 μM, less than about 0.2 μM, less than about 0.1 μM, or less than about 0.05 μM, as measured in a surface plasmon resonance assay.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

The term “K_{D}”, as used herein, means the equilibrium dissociation constant of a particular protein-protein interaction (e.g., antibody-antigen interaction). Unless indicated otherwise, the K_{D} values disclosed herein refer to K_{D} values determined by surface plasmon resonance assay at 25° C.

As indicated above, an “antigen-binding domain” (D1 and/or D2) may comprise or consist of an antibody or antigen-binding fragment of an antibody. The term “antibody,” as used herein, means any antigen-binding molecule or molecular complex comprising at least one complementarity determining region (CDR) that specifically binds to or interacts with a particular antigen (e.g., human MET). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the antibodies provided herein (or antigen-binding portion thereof) may be identical to the human germline sequences, or may be naturally or artificially modified. An amino acid consensus sequence may be defined based on a side-by-side analysis of two or more CDRs.

The D1 and/or D2 components of the bispecific antigen-binding molecules provided herein may comprise or consist of antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-CL; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The bispecific antigen-binding molecules useful in the methods provided herein may comprise or consist of human antibodies and/or recombinant human antibodies, or fragments thereof. The term “human antibody”, as used herein, includes antibodies having variable and constant regions derived from human germline immunoglobulin sequences. Human antibodies may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The bispecific antigen-binding molecules useful in the methods provided herein may comprise or consist of recombinant human antibodies or antigen-binding fragments thereof. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Methods for making bispecific antibodies are known in the art and may be used to construct bispecific antigen-binding molecules disclosed herein. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab^{e} bispecific formats (see, e.g., Klein et al. 2012, mAbs 4:6, 1-11, and references cited therein, for a review of the foregoing formats).

Exemplary antigen-binding domains (D1 and D2) that can be included in the MET×MET bispecific antigen-binding molecules provided herein include antigen-binding domains derived from any of the anti-MET antibodies disclosed herein. For example, MET×MET bispecific antigen-binding molecules comprising a D1 or D2 antigen-binding domain comprising an HCVR comprising an amino acid sequence selected from any of the HCVR amino acid sequences listed in Table 1, or a substantially similar sequence thereof having at least 90%, at least 95%, at least 98% or at least 99% sequence identity thereto, are useful in the methods of treating uveal melanoma as described herein.

Also useful according to the methods provided herein are MET×MET bispecific antigen-binding molecules comprising a D1 or D2 antigen-binding domain comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

Also useful according to the methods provided herein are MET×MET bispecific antigen-binding molecules comprising a D1 or D2 antigen-binding domain comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, useful MET×MET bispecific antigen-binding molecules comprise a D1 or D2 antigen-binding domain comprising an HCVR/LCVR amino acid sequence pair contained within any of the exemplary anti-MET antibodies listed in Table 1.

Also useful according to the methods provided herein are MET×MET bispecific antigen-binding molecules comprising a D1 or D2 antigen-binding domain comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

Also useful according to the methods provided herein are MET×MET bispecific antigen-binding molecules comprising a D1 or D2 antigen-binding domain comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

Also useful according to the methods provided herein are MET×MET bispecific antigen-binding molecules comprising a D1 or D2 antigen-binding domain comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

Also useful according to the methods provided herein are MET×MET bispecific antigen-binding molecules comprising a D1 or D2 antigen-binding domain comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

Also useful according to the methods provided herein are MET×MET bispecific antigen-binding molecules comprising a D1 or D2 antigen-binding domain comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

Also useful according to the methods provided herein are MET×MET bispecific antigen-binding molecules comprising a D1 or D2 antigen-binding domain comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

Also useful according to the methods provided herein are MET×MET bispecific antigen-binding molecules comprising a D1 or D2 antigen-binding domain comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1. According to certain embodiments, the present disclosure provides antibodies, or antigen-binding fragments thereof, comprising an HCDR3/LCDR3 amino acid sequence pair contained within any of the exemplary anti-MET antibodies listed in Table 1.

Also useful according to the methods provided herein are MET×MET bispecific antigen-binding molecules comprising a D1 or D2 antigen-binding domain comprising a set of six CDRs (i.e., HCDR1-HCDR2-HCDR3-LCDR1-LCDR2-LCDR3) contained within any of the exemplary anti-MET antibodies listed in Table 1.

Also useful according to the methods provided herein are MET×MET bispecific antigen-binding molecules comprising a D1 or D2 antigen-binding domain comprising a set of six CDRs (i.e., HCDR1-HCDR2-HCDR3-LCDR1-LCDR2-LCDR3) contained within an HCVR/LCVR amino acid sequence pair as defined by any of the exemplary anti-MET antibodies listed in Table 1.

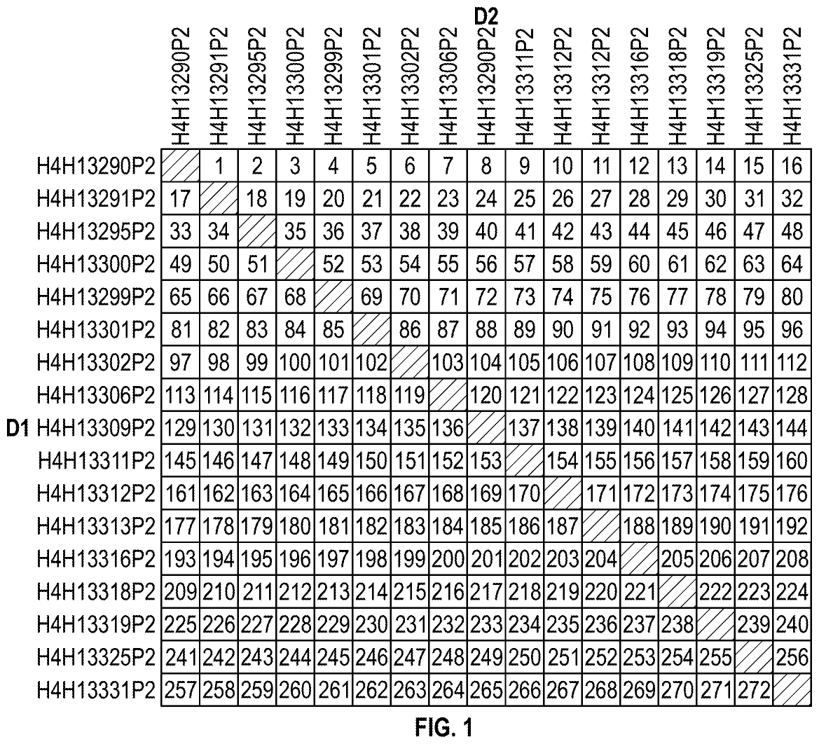

The MET×MET bispecific antigen-binding molecules useful in the methods provided herein may comprise a D1 antigen-binding domain derived from any of the anti-MET antibodies of Table 1, and a D2 antigen-binding domain derived from any other anti-MET antibody of Table 1. Non-limiting examples of MET×MET bispecific antibodies are depicted in FIG. 1. FIG. 1 is a matrix illustrating the components of 272 exemplary MET×MET bispecific antibodies. Each numbered cell of the matrix (numbered 1 through 272) identifies a unique bispecific antibody comprising a “D1” antigen binding domain and a “D2” antigen binding domain, wherein the D1 antigen binding domain comprises the immunoglobulin variable domain (HCVR/LCVR amino acid sequence pair) or CDRs from the corresponding anti-MET antibody listed along the Y-axis, and wherein the D2 antigen binding domain comprises the immunoglobulin variable domain (HCVR/LCVR amino acid sequence pair) or CDRs from the corresponding anti-MET antibody listed along the X-axis. Thus, for example, the MET×MET bispecific antigen-binding molecule “number 10” shown in the matrix comprises a D1 antigen-binding domain comprising an HCVR/LCVR pair, or 6-CDR set, from the exemplary anti-MET antibody H4H13290P2, and a D2 antigen-binding domain comprising an HCVR/LCVR pair, or 6-CDR set, from the exemplary anti-MET antibody H4H13321P2. Additional examples of MET×MET bispecific antibodies provided herein are described in Example 4 herein.

An exemplary MET×MET bispecific antigen binding molecule useful according to the methods provided herein comprises a D1 antigen-binding domain and a D2 antigen-binding domain, wherein the D1 antigen binding domain comprises an HCVR/LCVR amino acid sequence pair of SEQ ID NOs: 58/138, or a set of heavy and light chain CDRs (HCDR1-HCDR2-HCDR3-LCDR1-LCDR2-LCDR3) comprising SEQ ID NOs: 60-62-64-140-142-144, and wherein the D2 antigen-binding domain comprises an HCVR/LCVR amino acid sequence pair of SEQ ID NOs: 82/138, or a set of heavy and light chain CDRs (HCDR1-HCDR2-HCDR3-LCDR1-LCDR2-LCDR3) comprising SEQ ID NOs: 84-86-88-140-142-144. An exemplary MET×MET bispecific antibody having these sequence characteristics is the bispecific antibody designated H4H14639D, also referred to as bispecific antibody No. 122, which comprises a D1 derived from H4H13306P2 and a D2 derived from H4H13312P2 (see Example 4, Table 5 herein).

As a further non-limiting illustrative example, the MET×MET bispecific antigen binding molecules useful herein comprise a D1 antigen-binding domain and a D2 antigen-binding domain, wherein the D1 antigen binding domain comprises an HCVR/LCVR amino acid sequence pair of SEQ ID NOs: 18/138, or a set of heavy and light chain CDRs (HCDR1-HCDR2-HCDR3-LCDR1-LCDR2-LCDR3) comprising SEQ ID NOs: 20-22-24-140-142-144, and wherein the D2 antigen-binding domain comprises an HCVR/LCVR amino acid sequence pair of SEQ ID NOs: 82/138, or a set of heavy and light chain CDRs (HCDR1-HCDR2-HCDR3-LCDR1-LCDR2-LCDR3) comprising SEQ ID NOs: 84-86-88-140-142-144. An exemplary MET×MET bispecific antibody having these sequence characteristics is the bispecific antibody designated H4H14635D, also referred to as bispecific antibody No. 42, which comprises a D1 derived from H4H13295P2 and a D2 derived from H4H13312P2 (see Example 4, Table 5 herein).

### Multimerizing Components

The bispecific antigen-binding molecules useful according to the methods provided herein may, in certain embodiments, also comprise one or more multimerizing component(s). The multimerizing components can function to maintain the association between the antigen-binding domains (D1 and D2). As used herein, a “multimerizing component” is any macromolecule, protein, polypeptide, peptide, or amino acid that has the ability to associate with a second multimerizing component of the same or similar structure or constitution. For example, a multimerizing component may be a polypeptide comprising an immunoglobulin C_{H}3 domain. A non-limiting example of a multimerizing component is an Fc portion of an immunoglobulin, e.g., an Fc domain of an IgG selected from the isotypes IgG1, IgG2, IgG3, and IgG4, as well as any allotype within each isotype group. In certain embodiments, the multimerizing component is an Fc fragment or an amino acid sequence of 1 to about 200 amino acids in length containing at least one cysteine residues. In other embodiments, the multimerizing component is a cysteine residue, or a short cysteine-containing peptide. Other multimerizing domains include peptides or polypeptides comprising or consisting of a leucine zipper, a helix-loop motif, or a coiled-coil motif.

In certain embodiments, the bispecific antigen-binding molecules comprise two multimerizing domains, M1 and M2, wherein D1 is attached to M1 and D2 is attached to M2, and wherein the association of M1 with M2 facilitates the physical linkage of D1 and D2 to one another in a single bispecific antigen-binding molecule. In certain embodiments, M1 and M2 are identical to one another. For example, M1 can be an Fc domain having a particular amino acid sequence, and M2 is an Fc domain with the same amino acid sequence as M1. Alternatively, M1 and M2 may differ from one another at one or more amino acid position. For example, M1 may comprise a first immunoglobulin (Ig) C_{H}3 domain and M2 may comprise a second Ig C_{H}3 domain, wherein the first and second Ig C_{H}3 domains differ from one another by at least one amino acid, and wherein at least one amino acid difference reduces binding of the targeting construct to Protein A as compared to a reference construct having identical M1 and M2 sequences. In one embodiment, the Ig C_{H}3 domain of M1 binds Protein A and the Ig C_{H}3 domain of M2 contains a mutation that reduces or abolishes Protein A binding such as an H95R modification (by IMGT exon numbering; H435R by EU numbering). The C_{H}3 of M2 may further comprise a Y96F modification (by IMGT; Y436F by EU). Further modifications that may be found within the C_{H}3 of M2 include: D16E, L18M, N44S, K52N, V57M, and V82I (by IMGT; D356E, L358M, N384S, K392N, V397M, and V422I by EU) in the case of an IgG1 Fc domain; N44S, K52N, and V82I (IMGT; N384S, K392N, and V422I by EU) in the case of an IgG2 Fc domain; and Q15R, N44S, K52N, V57M, R69K, E79Q, and V82I (by IMGT; Q355R, N384S, K392N, V397M, R409K, E419Q, and V422I by EU) in the case of an IgG4 Fc domain.

The bispecific antigen-binding molecules useful according to the methods provided herein may be “isolated.” An “isolated bispecific antigen-binding molecule,” as used herein, means a bispecific antigen-binding molecule that has been identified and separated and/or recovered from at least one component of its natural environment. For example, a bispecific antibody that has been separated or removed from at least one component of an organism, or from a tissue or cell in which the antibody is produced, is an “isolated bispecific antibody” for purposes of the present disclosure. An isolated bispecific antigen-binding molecule also includes molecules in situ within a recombinant cell. Isolated bispecific antigen-binding molecules are molecules that have been subjected to at least one purification or isolation step. According to certain embodiments, an isolated bispecific antigen-binding molecule may be substantially free of other cellular material and/or chemicals.

The bispecific antigen-binding molecules useful according to the methods provided herein, or the antigen-binding domains thereof (D1 and/or D2) may comprise one or more amino acid substitutions, insertions and/or deletions in the framework and/or CDR regions of the heavy and light chain variable domains as compared to the corresponding germline sequences from which the antigen-binding proteins or antigen-binding domains were derived. Such mutations can be readily ascertained by comparing the amino acid sequences disclosed herein to germline sequences available from, for example, public antibody sequence databases. The bispecific antigen-binding molecules, or the antigen-binding domains thereof (D1 and/or D2), are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”).

A person of ordinary skill in the art, starting with the heavy and light chain variable region sequences disclosed herein, can easily produce numerous bispecific antigen-binding molecules, or antigen-binding domains thereof (D1 and/or D2), which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived).

Furthermore, the bispecific antigen-binding molecules, or the antigen-binding domains thereof (D1 and/or D2), useful herein, may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, bispecific antigen-binding molecules, or the antigen-binding domains thereof (D1 and/or D2), that contain one or more germline mutations can be easily tested for one or more desired property such as, improved binding specificity, increased binding affinity, improved or enhanced antagonistic or agonistic biological properties (as the case may be), reduced immunogenicity, etc. Bispecific antigen-binding molecules, or the antigen-binding domains thereof (D1 and/or D2), obtained in this general manner are contemplated as useful herein.

### Variants

Also useful herein are anti-MET antibodies and bispecific antigen-binding molecules comprising variants of any of the HCVR, LCVR, and/or CDR amino acid sequences disclosed herein. Exemplary variants included within this aspect include variants of any of the HCVR, LCVR, and/or CDR amino acid sequences disclosed herein having one or more conservative substitutions. For example, the present disclosure includes anti-MET antibodies and MET×MET bispecific antigen-binding molecules having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in Table 1 herein.

Exemplary variants include variants having substantial sequence identity to any of the HCVR, LCVR, and/or CDR amino acid sequences disclosed herein. As used herein in the context of amino acid sequences, the term “substantial identity” or “substantially identical” means that two amino acid sequences, when optimally aligned, such as by the programs GAP or BESTFIT using default gap weights, share at least 95%, 98% or 99% sequence identity. In certain embodiments, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence identity between two different amino acid sequences is typically measured using sequence analysis software. Sequence analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence provided herein to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

### Anti-MET Antibodies and MET×MET Bispecific Antigen-Binding Molecules Comprising FC Variants

According to certain embodiments provided herein, anti-MET antibodies and MET×MET bispecific antigen binding proteins useful herein comprise an Fc domain comprising one or more mutations which enhance or diminish antibody binding to the FcRn receptor, e.g., at acidic pH as compared to neutral pH. For example, such variants include anti-MET antibodies and MET×MET bispecific antigen binding proteins comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

Accordingly, useful herein are anti-MET antibodies and MET×MET bispecific antigen binding proteins comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). All possible combinations of the foregoing Fc domain mutations, and other mutations within the antibody variable domains disclosed herein, are contemplated within the scope of the present disclosure.

### Biological Characteristics of the Antigen-Binding Molecules Useful Herein

Useful according to the methods provided herein are antibodies and antigen-binding fragments thereof, as well as ADCs comprising the antibodies and antigen-binding fragments, that inhibit proliferation, inhibit invasion, cause apoptosis, and/or decrease viability of an uveal melanoma cell.

Also useful according to the methods provided herein are antibodies and antigen-binding fragments thereof, as well as ADCs comprising the antibodies and antigen-binding fragments, that affect the cell cycle of an uveal melanoma cell. In some aspects, the cell undergoes mitotic arrest. In some aspects, the cell remains in a SubG1 phase, indicative that the cell is undergoing apoptosis.

Also useful according to the methods provided herein are antibodies and antigen-binding fragments thereof, as well as ADCs comprising the antibodies and antigen-binding fragments, that cause apoptosis in a uveal melanoma cell. In some aspects, the uveal melanoma cell demonstrates PARP cleavage. In some aspects, the uveal melanoma cell demonstrates histone H3 phosphorylation.

Also useful according to the methods provided herein are antibodies and antigen-binding fragments thereof that bind monomeric human MET with high affinity. For example, the present disclosure includes anti-MET antibodies that bind monomeric human MET (e.g., hMET.mmh) with a K_{D} of less than about 230 nM as measured by surface plasmon resonance at 25° C. or 37° C., e.g., using an assay format as defined in Example 3 herein, or a substantially similar assay. According to certain embodiments, anti-MET antibodies are provided that bind monomeric human MET at 37° C. with a K_{D} of less than about 230 nM, less than about 200 nM, less than about 150 nM, less than about 100 nM, less than about 50 nM, less than about 25 nM, less than about 20 nM, less than about 10 nM, Less than about 8 nM, less than about 6 nM, less than about 5 nM, less than about 4 nM, or less than about 3 nM, as measured by surface plasmon resonance, e.g., using an assay format as defined in Example 3 herein, or a substantially similar assay.

Such antibodies and antigen-binding fragments thereof bind monomeric human MET (e.g., hMET.mmh) with a dissociative half-life (t1/2) of greater than about 1 minute as measured by surface plasmon resonance at 25° C. or 37° C., e.g., using an assay format as defined in Example 3 herein, or a substantially similar assay. Such anti-MET antibodies bind monomeric human MET at 37° C. with a t1/2 of greater than about 1 minute, greater than about 2 minutes, greater than about 4 minutes, greater than about 6 minutes, greater than about 8 minutes, greater than about 10 minutes, greater than about 12 minutes, greater than about 14 minutes, greater than about 16 minutes, greater than about 18 minutes, or greater than about 20 minutes, or longer, as measured by surface plasmon resonance, e.g., using an assay format as defined in Example 3 herein, or a substantially similar assay.

Such antibodies and antigen-binding fragments thereof bind dimeric human MET (e.g., hMET.mFc) with high affinity. For example, the anti-MET antibodies bind dimeric human MET with a K_{D} of less than about 3 nM as measured by surface plasmon resonance at 25° C. or 37° C., e.g., using an assay format as defined in Example 3 herein, or a substantially similar assay. According to certain embodiments, anti-MET antibodies bind dimeric human MET at 37° C. with a K_{D} of less than about 3 nM, less than about 2 nM, less than about 1 nM, less than about 0.9 nM, less than about 0.8 nM, less than about 0.7 nM, less than about 0.6 nM, less than about 0.5 nM, less than about 0.4 nM, less than about 0.3 nM, or less than about 0.25 nM, as measured by surface plasmon resonance, e.g., using an assay format as defined in Example 3 herein, or a substantially similar assay.

Such antibodies and antigen-binding fragments thereof may bind dimeric human MET (e.g., hMET.mFc) with a dissociative half-life (t1/2) of greater than about 4 minutes as measured by surface plasmon resonance at 25° C. or 37° C., e.g., using an assay format as defined in Example 3 herein, or a substantially similar assay. According to certain embodiments, anti-MET antibodies may bind dimeric human MET at 37° C. with a t1/2 of greater than about 4 minutes, greater than about 5 minutes, greater than about 10 minutes, greater than about 20 minutes, greater than about 30 minutes, greater than about 40 minutes, greater than about 50 minutes, greater than about 60 minutes, greater than about 70 minutes, greater than about 80 minutes, greater than about 90 minutes, greater than about 100 minutes, greater than about 105 minutes, or longer, as measured by surface plasmon resonance, e.g., using an assay format as defined in Example 3 herein, or a substantially similar assay.

Also useful according to the methods provided herein are MET×MET bispecific antigen-binding proteins that bind dimeric human MET (e.g., hMET.mFc) with a dissociative half-life (t1/2) of greater than about 10 minutes as measured by surface plasmon resonance at 25° C. or 37° C., e.g., using an assay format as defined in Example 5 herein, or a substantially similar assay. According to certain embodiments, MET×MET bispecific antigen-binding proteins bind dimeric human MET at 37° C. with a t1/2 of greater than about 10 minutes, greater than about 20 minutes, greater than about 30 minutes, greater than about 40 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, greater than about 1100 minutes, or longer, as measured by surface plasmon resonance, e.g., using an assay format as defined in Example 5 herein, or a substantially similar assay.

Also according to the methods provided herein are anti-MET antibodies and MET×MET bispecific antigen-binding proteins that block the interaction between HGF and MET, e.g., in an in vitro ligand-binding assay. According to certain embodiments provided herein, MET×MET bispecific antigen-binding proteins block HGF binding to cells expressing human MET, and induce minimal or no MET activation in the absence of HGF signaling. For example, the MET×MET bispecific antigen-binding proteins exhibit a degree of MET agonist activity in a cell-based MET activity reporter assay that is less than 50%, less than 40%, less than 30%, less than 20%, less than 10%, less than 5%, less than 3%, less than 2% or less than 1% of the MET agonist activity observed in an equivalent activity reporter assay using a monospecific antibody comprising D1 or D2 alone.

The antibodies and antigen-binding proteins useful according to the present disclosure may possess one or more of the aforementioned biological characteristics, or any combination thereof. The foregoing list of biological characteristics of the antibodies is not intended to be exhaustive. Other biological characteristics of the antibodies provided herein will be evident to a person of ordinary skill in the art from a review of the present disclosure including the working Examples herein.

### Antibody-Drug Conjugates (ADCs)

Useful according to the methods provided herein are antibody-drug conjugates (ADCs) comprising an anti-MET antibody or a MET×MET bispecific antigen-binding protein conjugated to a therapeutic moiety such as a cytotoxic agent, a chemotherapeutic drug, or a radioisotope.

Cytotoxic agents include any agent that is detrimental to the growth, viability or propagation of cells, including, but not limited to, tubulin-interacting agents and DNA-damaging agents. Examples of suitable cytotoxic agents and chemotherapeutic agents that can be conjugated to anti-MET antibodies in accordance with this aspect of the disclosure include, e.g., 1-(2chloroethyl)-1,2-dimethanesulfonyl hydrazide, 1,8-dihydroxy-bicyclo[7.3.1]trideca-4,9-diene-2,6-diyne-13-one, 1-dehydrotestosterone, 5-fluorouracil, 6-mercaptopurine, 6-thioguanine, 9-amino camptothecin, actinomycin D, amanitins, aminopterin, anguidine, anthracycline, anthramycin (AMC), auristatins, bleomycin, busulfan, butyric acid, calicheamicins (e.g., calicheamicin γ₁), camptothecin, carminomycins, carmustine, cemadotins, cisplatin, colchicin, combretastatins, cyclophosphamide, cytarabine, cytochalasin B, dactinomycin, daunorubicin, decarbazine, diacetoxypentyldoxorubicin, dibromomannitol, dihydroxy anthracin dione, disorazoles, dolastatin (e.g., dolastatin 10), doxorubicin, duocarmycin, echinomycins, eleutherobins, emetine, epothilones, esperamicin, estramustines, ethidium bromide, etoposide, fluorouracils, geldanamycins, gramicidin D, glucocorticoids, irinotecans, kinesin spindle protein (KSP) inhibitors, leptomycins, leurosines, lidocaine, lomustine (CCNU), maytansinoids, mechlorethamine, melphalan, mercatopurines, methopterins, methotrexate, mithramycin, mitomycin, mitoxantrone, N8-acetyl spermidine, podophyllotoxins, procaine, propranolol, pteridines, puromycin, pyrrolobenzodiazepines (PBDs), rhizoxins, streptozotocin, tallysomycins, taxol, tenoposide, tetracaine, thioepa chlorambucil, tomaymycins, topotecans, tubulysin, vinblastine, vincristine, vindesine, vinorelbines, and derivatives of any of the foregoing. According to certain embodiments, the cytotoxic agent that is conjugated to an anti-MET antibody is a maytansinoid such as DM1 or DM4, a tomaymycin derivative, or a dolastatin derivative. According to certain embodiments, the cytotoxic agent that is conjugated to an anti-MET antibody is an auristatin such as MMAE, MMAF, or derivatives thereof. Other cytotoxic agents known in the art are contemplated within the scope of the present disclosure, including, e.g., protein toxins such ricin, C. difficile toxin, pseudomonas exotoxin, ricin, diphtheria toxin, botulinum toxin, bryodin, saporin, pokeweed toxins (i.e., phytolaccatoxin and phytolaccigenin), and others such as those set forth in Sapra et al., Pharmacol. & Therapeutics, 2013, 138:452-469.

In certain embodiments, the cytotoxic agent is a maytansinoid, e.g., derivative of maytansine. Suitable maytansinoids include DM1, DM4, or derivatives, stereoisomers, or isotopologues thereof. Suitable maytansinoids also include, but are not limited to, those disclosed in WO 2014/145090A1, WO 2015/031396A1, US 2016/0375147A1, and US 2017/0209591A1, incorporated herein by reference in their entireties.

In some embodiments, the maytansinoid has the following structure:

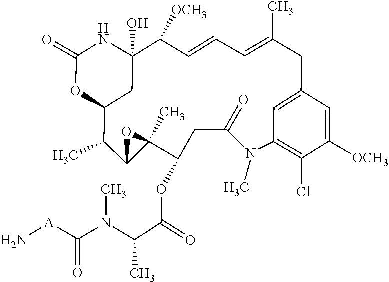

wherein A is an optionally substituted arylene or heteroarylene.

In some embodiments, the maytansinoid has the following structure:

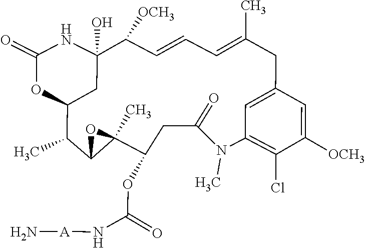

wherein A is an optionally substituted arylene or heteroarylene.

In some embodiments, the maytansinoid has the following structure:

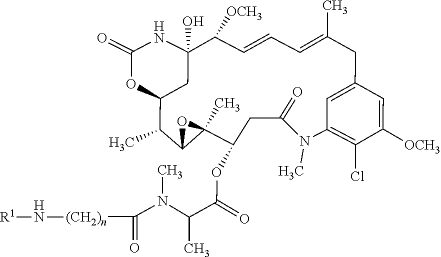

wherein n is an integer from 1-12 and R¹ is alkyl.

In some embodiments, the maytansinoid is:

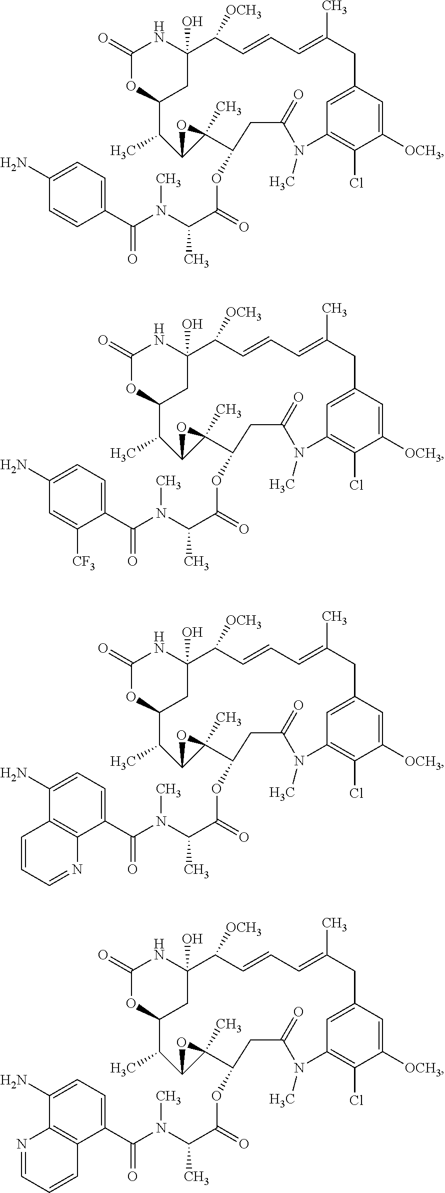

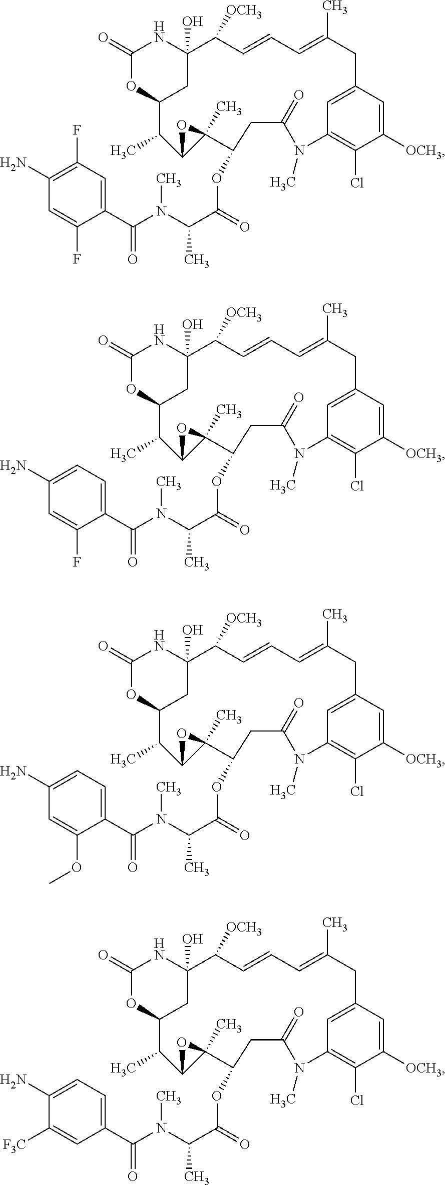

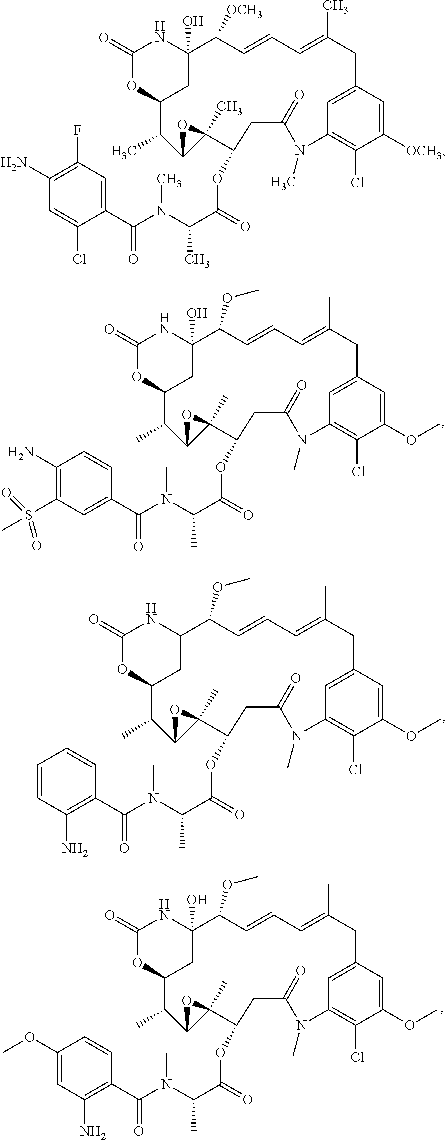

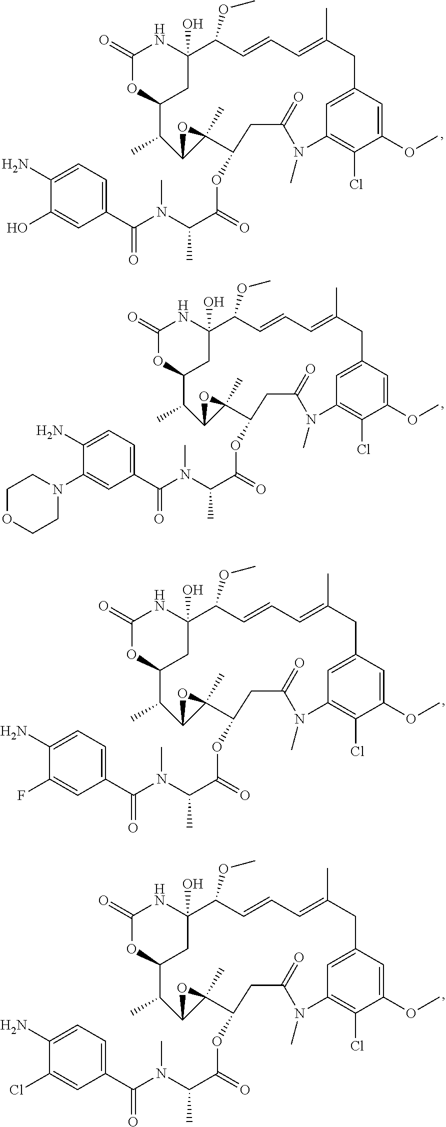

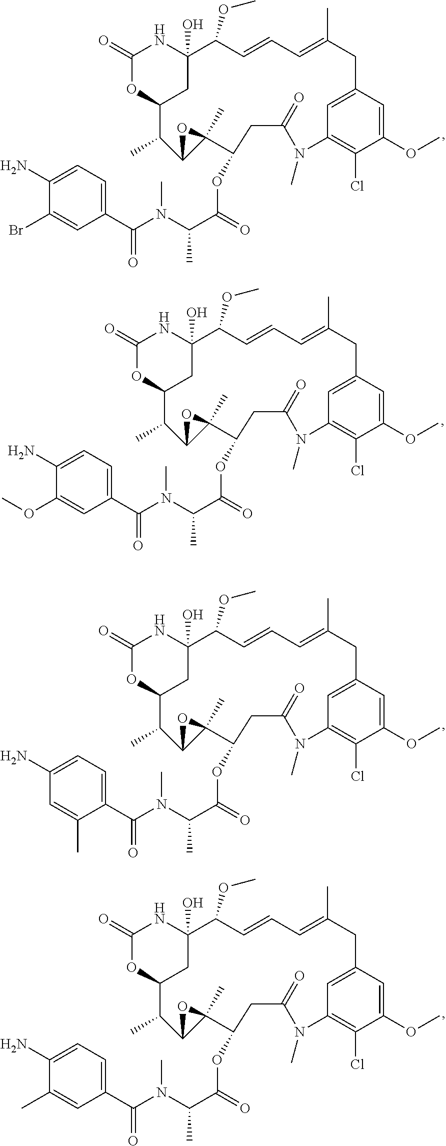

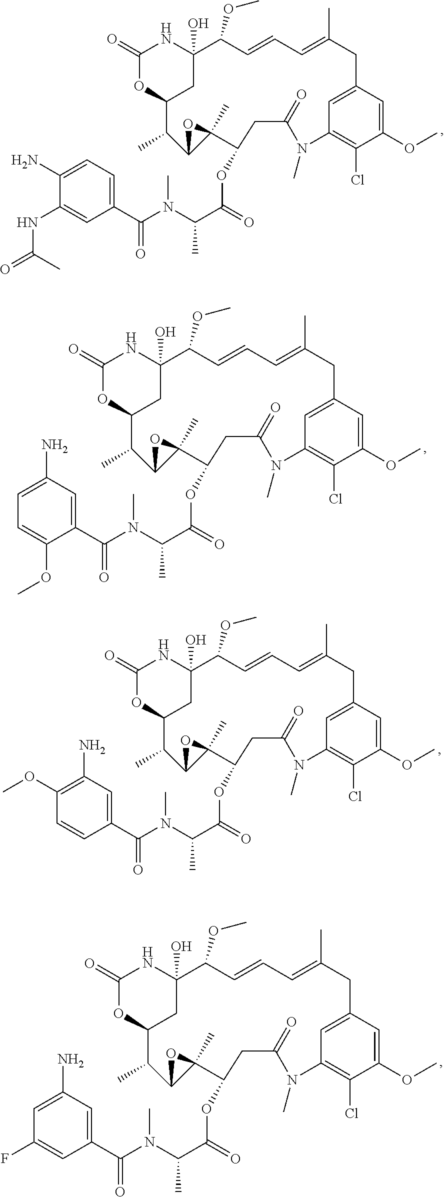

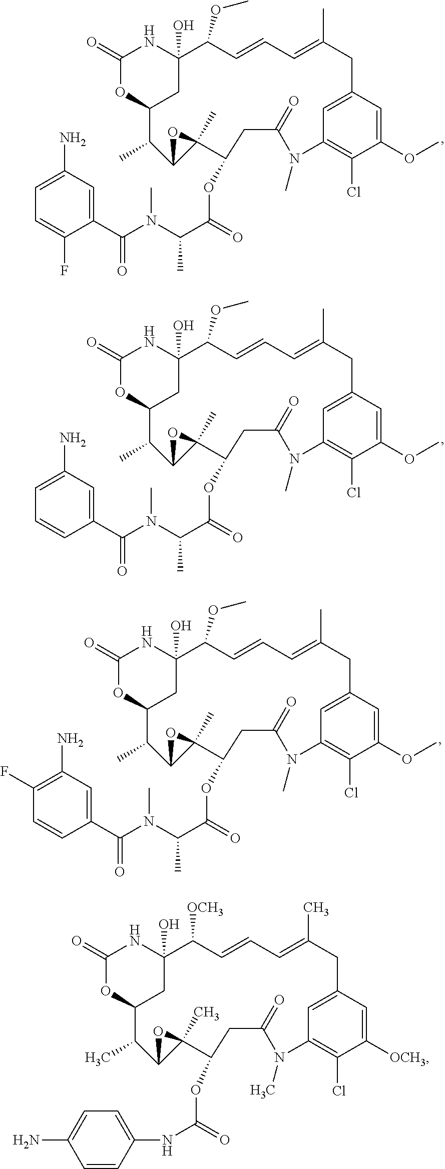

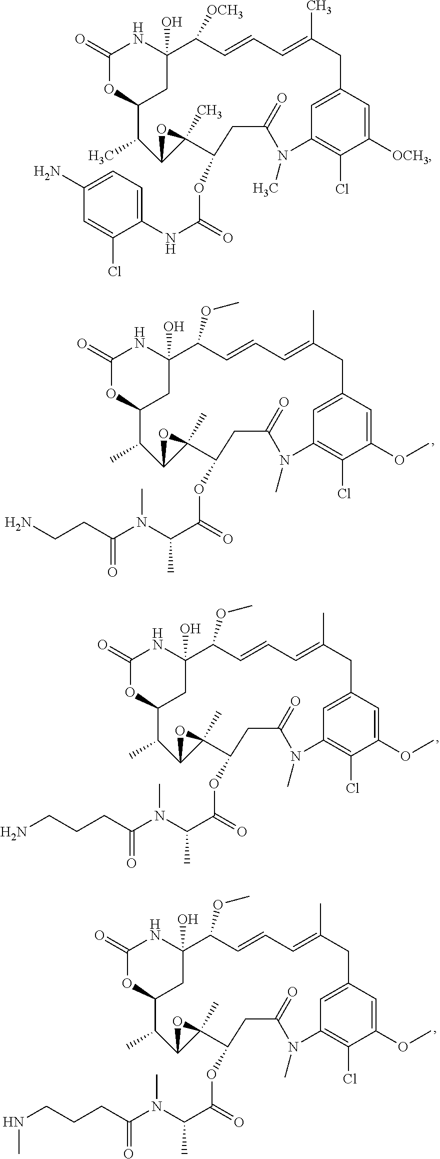

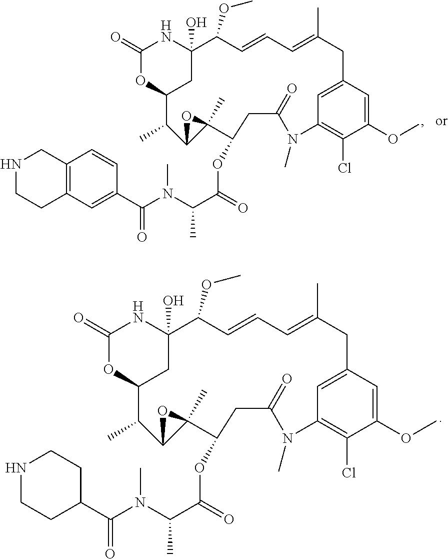 [SMILES: COc1cc2cc(c1Cl)N(C)C(=O)C[C@H](OC(=O)[C@H](C)N(C)C(=O)c1ccc3c(c1)CCNC3)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)C1CCNCC1)[C@]1(C)O[C@H]1[C@H](C)[C@@H]1C[C@@](O)(NC(=O)O1)[C@H](OC)/C=C/C=C(\C)C2]

In some embodiments, the maytansinoid is:

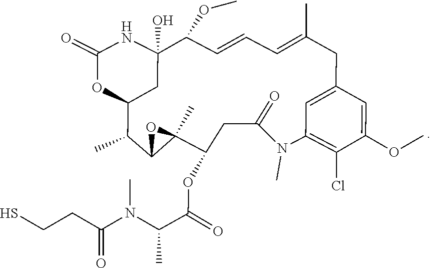 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2]

In some embodiments, the maytansinoid is:

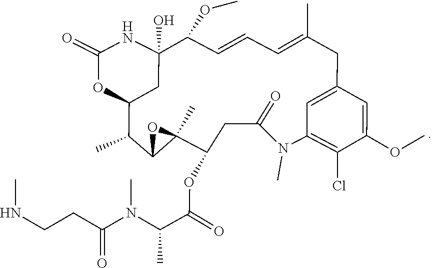 [SMILES: CNCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

Also useful according to the methods provided herein are antibody-radionuclide conjugates (ARCs) comprising anti-MET antibodies conjugated to one or more radionuclides. Exemplary radionuclides that can be used in the context of this aspect of the disclosure include, but are not limited to, e.g., ²²⁵Ac, ²¹²Bi, ²¹³Bi, ¹³¹I, ¹⁸⁶Re, ²²⁷Th, ²²²Rn, ²²³Ra, ²²⁴Ra, and ⁹⁰Y.

In certain embodiments, ADCs comprise an anti-MET antibody or a MET×MET bispecific antigen-binding protein conjugated to a cytotoxic agent (e.g., any of the cytotoxic agents disclosed above) via a linker molecule. Linkers are any group or moiety that links, connects, or bonds the antibody or antigen-binding proteins described herein with a therapeutic moiety, e.g. cytotoxic agent. Suitable linkers may be found, for example, in Antibody-Drug Conjugates and Immunotoxins; Phillips, G. L., Ed.; Springer Verlag: New York, 2013; Antibody-Drug Conjugates; Ducry, L., Ed.; Humana Press, 2013; Antibody-Drug Conjugates; Wang, J., Shen, W.-C., and Zaro, J. L., Eds.; Springer International Publishing, 2015, the contents of each incorporated herein in their entirety by reference. Generally, suitable binding agent linkers for the antibody conjugates described herein are those that are sufficiently stable to exploit the circulating half-life of the antibody and, at the same time, capable of releasing its payload after antigen-mediated internalization of the conjugate. Linkers can be cleavable or non-cleavable. Cleavable linkers include linkers that are cleaved by intracellular metabolism following internalization, e.g., cleavage via hydrolysis, reduction, or enzymatic reaction. Non-cleavable linkers include linkers that release an attached payload via lysosomal degradation of the antibody following internalization. Suitable linkers include, but are not limited to, acid-labile linkers, hydrolysis-labile linkers, enzymatically cleavable linkers, reduction labile linkers, self-immolative linkers, and non-cleavable linkers. Suitable linkers also include, but are not limited to, those that are or comprise peptides, glucuronides, succinimide-thioethers, polyethylene glycol (PEG) units, hydrazones, mal-caproyl units, dipeptide units, valine-citruline units, and para-aminobenzyl (PAB) units.

Any linker molecule or linker technology known in the art can be used to create or construct an ADC useful according to the present disclosure. In certain embodiments, the linker is a cleavable linker. According to other embodiments, the linker is a non-cleavable linker. Exemplary linkers that can be used in the context of the present disclosure include, linkers that comprise or consist of e.g., MC (6-maleimidocaproyl), MP (maleimidopropanoyl), val-cit (valine-citrulline), val-ala (valine-alanine), dipeptide site in protease-cleavable linker, ala-phe (alanine-phenylalanine), dipeptide site in protease-cleavable linker, PAB (p-aminobenzyloxycarbonyl), SPP (N-Succinimidyl 4-(2-pyridylthio) pentanoate), SMCC (N-Succinimidyl 4-(N-maleimidomethyl)cyclohexane-1 carboxylate), SIAB (N-Succinimidyl (4-iodo-acetyl)aminobenzoate), and variants and combinations thereof. Additional examples of linkers that can be used in the context of the present disclosure are provided, e.g., in U.S. Pat. No. 7,754,681 and in Ducry, Bioconjugate Chem., 2010, 21:5-13, and the references cited therein, the contents of which are incorporated by reference herein in their entireties.

In certain embodiments, the linkers are stable in physiological conditions. In certain embodiments, the linkers are cleavable, for instance, able to release at least the payload portion in the presence of an enzyme or at a particular pH range or value. In some embodiments, a linker comprises an enzyme-cleavable moiety. Illustrative enzyme-cleavable moieties include, but are not limited to, peptide bonds, ester linkages, hydrazones, and disulfide linkages. In some embodiments, the linker comprises a cathepsin-cleavable linker.

In some embodiments, the linker comprises a non-cleavable moiety.

Suitable linkers also include, but are not limited to, those that are chemically bonded to two cysteine residues of a single binding agent, e.g., antibody. Such linkers can serve to mimic the antibody's disulfide bonds that are disrupted as a result of the conjugation process.

In some embodiments, the linker comprises one or more amino acids. Suitable amino acids include natural, non-natural, standard, non-standard, proteinogenic, non-proteinogenic, and L- or D-a-amino acids. In some embodiments, the linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or combination thereof. In certain embodiments, one or more side chains of the amino acids is linked to a side chain group, described below. In some embodiments, the linker comprises valine and citrulline. In some embodiments, the linker comprises lysine, valine, and citrulline. In some embodiments, the linker comprises lysine, valine, and alanine. In some embodiments, the linker comprises valine and alanine.

In some embodiments, the linker comprises a self-immolative group. The self-immolative group can be any such group known to those of skill. In particular embodiments, the self-immolative group is p-aminobenzyl (PAB), or a derivative thereof. Useful derivatives include p-aminobenzyloxycarbonyl (PABC). Those of skill will recognize that a self-immolative group is capable of carrying out a chemical reaction which releases the remaining atoms of a linker from a payload.

In some embodiments, the linker is:

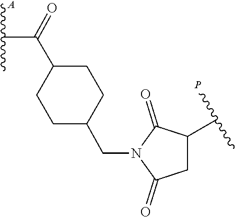

wherein [symbol] is a bond to the antibody or antigen-binding protein (e.g., via lysine residue) and [symbol] is a bond to the cytotoxic agent (e.g., DM1). In some embodiments, the linker is:

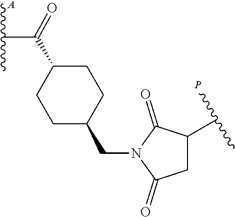

wherein [symbol] is a bond to the antibody or antigen-binding protein (e.g., via lysine residue) and [symbol] is a bond to the cytotoxic agent (e.g., DM1). In certain embodiments, the linker is:

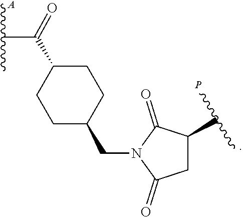

In certain embodiments, the linker is:

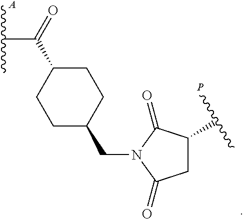

In some embodiments, the linker is derived from maleimidylmethyl-4-trans-cyclohexanecarboxysuccinate:

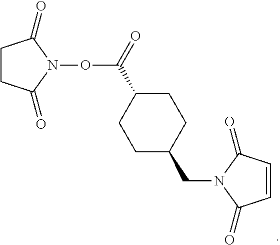 [SMILES: O=C1C=CC(=O)N1C[C@H]1CC[C@H](C(=O)ON2C(=O)CCC2=O)CC1]

In some embodiments, the linker is:

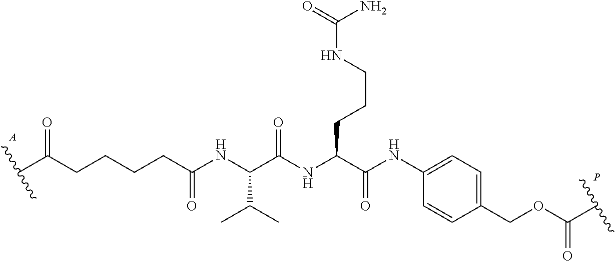

wherein [symbol] is a bond to the antibody or antigen-binding protein (e.g., via lysine residue) and [symbol] is a bond to the cytotoxic agent (e.g., a compound having the following formula:

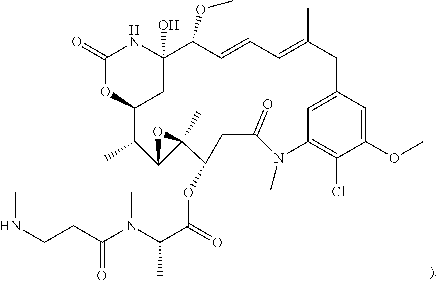 [SMILES: CNCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

Molecules useful according to the disclosed methods comprise ADCs in which a linker connects an anti-MET antibody or a MET×MET bispecific antigen-binding protein to a drug or cytotoxin through an attachment at a particular amino acid within the antibody or antigen-binding molecule. Exemplary amino acid attachments that can be used in the context of this aspect, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; US 2013/0101546; and US 2012/0585592), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenocysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Linkers can also be conjugated to an antigen-binding protein via attachment to carbohydrates (see, e.g., US 2008/0305497, WO 2014/065661, and Ryan et al., Food & Agriculture Immunol., 2001, 13:127-130) and disulfide linkers (see, e.g., WO 2013/085925, WO 2010/010324, WO 2011/018611, and Shaunak et al., Nat. Chem. Biol., 2006, 2:312-313). Site specific conjugation techniques can also be employed to direct conjugation to particular residues of the antibody or antigen binding protein (see, e.g., Schumacher et al. J Clin Immunol (2016) 36(Suppl 1): 100). Site specific conjugation techniques, include, but are not limited to glutamine conjugation via transglutaminase (see e.g., Schibli, Angew Chemie Inter Ed. 2010, 49, 9995).

According to certain embodiments, ADCs useful according to the methods provided herein comprise an anti-MET antibody or a MET×MET bispecific antigen-binding protein conjugated to a linker-drug composition as set forth in International Patent Publication WO2014/145090, (e.g., compound “7,” also referred to herein as “M0026” and depicted below), the disclosure of which is hereby incorporated by reference herein in its entirety:

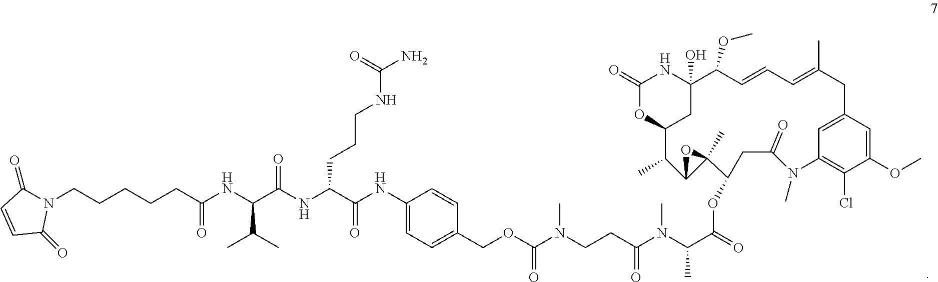 [SMILES: COc1cc2cc(c1Cl)N(C)C(=O)C[C@H](OC(=O)[C@H](C)N(C)C(=O)CCN(C)C(=O)OCc1ccc(NC(=O)[C@@H](CCCNC(N)=O)NC(=O)[C@H](NC(=O)CCCCCN3C(=O)C=CC3=O)C(C)C)cc1)[C@]1(C)O[C@H]1[C@H](C)[C@@H]1C[C@@](O)(NC(=O)O1)[C@H](OC)/C=C/C=C(\C)C2]

Also useful according to the methods provided herein are antibody-drug conjugates comprising the monospecific anti-MET antibodies and MET×MET bispecific antibodies, where said anti-MET antibody or MET×MET bispecific antibody is conjugated to a cytotoxic agent. In certain embodiments, the cytotoxic agent is a maytansinoid. In certain embodiments, the maytansinoid is a compound having the following formula:

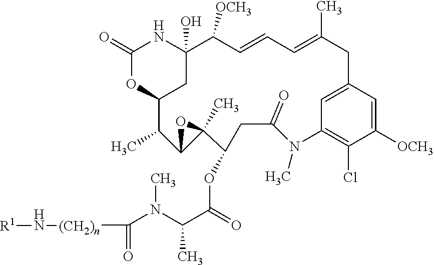

wherein n is an integer from 1-12 and R¹ is alkyl. In certain embodiments, the maytansinoid is

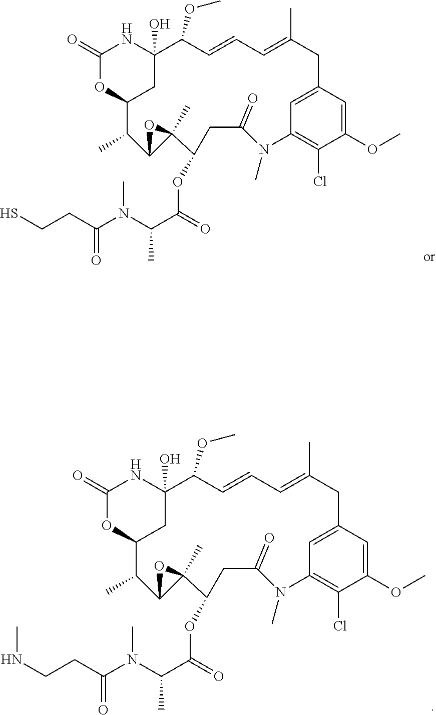 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2 ; CNCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In certain embodiments, the cytotoxic agent is a maytansinoid, and the maytansinoid is covalently attached to the antibody via non-cleavable linker. In certain embodiments, the cytotoxic agent is a maytansinoid, and the maytansinoid is covalently attached to the antibody via cleavable linker.

In one embodiment, the antibody is conjugated to:

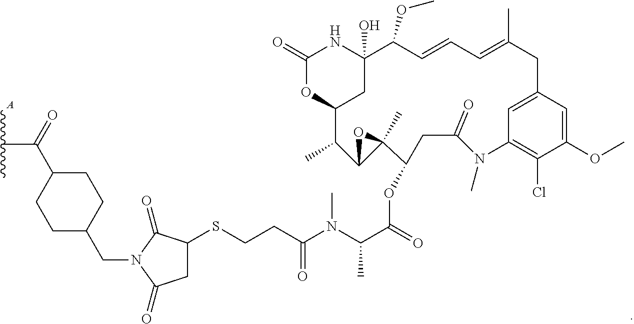

wherein [symbol] is a bond to the antibody.

In one embodiment, the antibody is conjugated to:

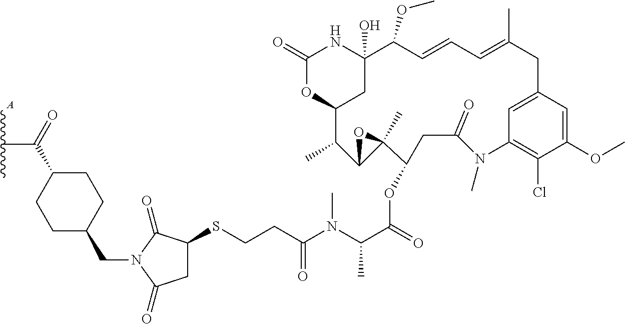

wherein [symbol] is a bond to the antibody.

In one embodiment, the antibody is conjugated to:

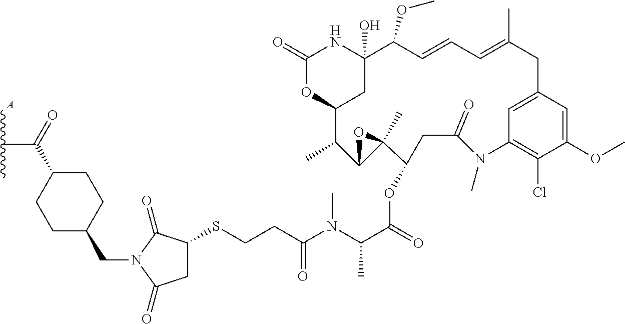

wherein [symbol] is a bond to the antibody.

In one embodiment, the antibody is conjugated to:

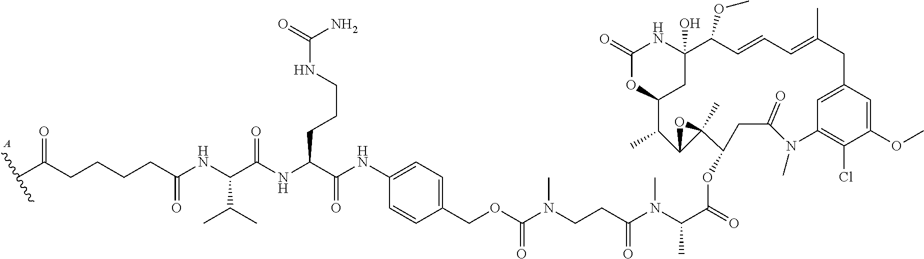

wherein [symbol] is a bond to the antibody.

In one embodiment, the antibody is conjugated to a diastereomer of a compound having the following structure

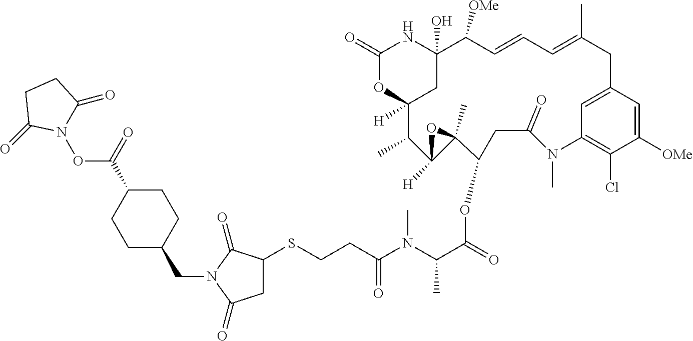 [SMILES: COc1cc2cc(c1Cl)N(C)C(=O)C[C@H](OC(=O)[C@H](C)N(C)C(=O)CCSC1CC(=O)N(C[C@H]3CC[C@H](C(=O)ON4C(=O)CCC4=O)CC3)C1=O)[C@]1(C)O[C@H]1[C@H](C)[C@@H]1C[C@@](O)(NC(=O)O1)[C@H](OC)/C=C/C=C(\C)C2]

wherein the diastereomer is characterized by a ¹H NMR characterized by delta shifts of (300 MHz, CDCl₃) δ 6.85 (d, 1H, J=4 Hz), 6.72 (m, 1H), 6.65 (d, 1H, J=4 Hz), 6.44 (dd, 1H, J=15 Hz, 11 Hz), 6.25 (s, 1H), 5.67 (dd, 1H, J=16 Hz, 9 Hz), 5.41 (m, 1H), 4.79 (d, 1H, J=11 Hz), 4.30 (t, 1H, J=11 Hz), 3.72 (m, 2H), 3.51 (d, 1H, J=9 Hz), 3.37 (m, 4H), 3.27 (m, 1H), 3.23 (s, 3H), 3.16-2.99 (m, 4H), 2.85 (m, 7H), 2.62 (m, 3H), 2.39 (ddd, 1H, J=19 Hz, 12 Hz, 4 Hz), 2.18 (br m, 2H), 1.77 (br m, 3H), 1.66 (s, 3H), 1.60-1.47 (m, 4H), 1.31 (m, 6H), 1.05 (m, 2H), and 0.82 (s, 3H).

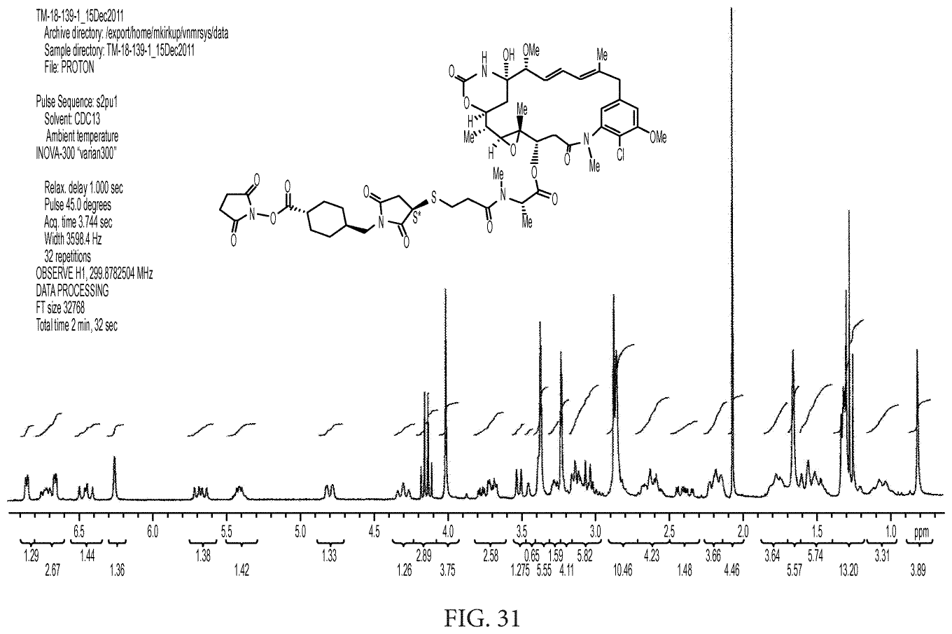

In one embodiment, the antibody is conjugated to a diastereomer of a compound having the following structure

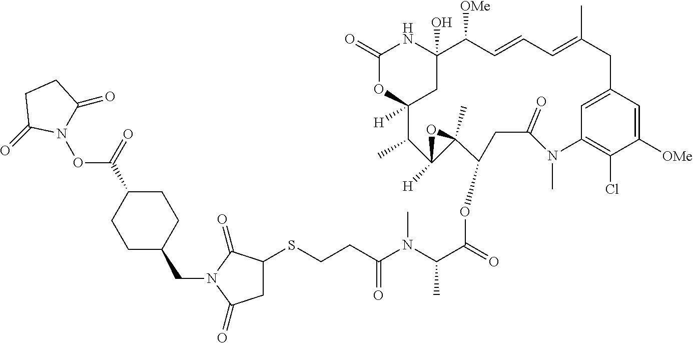 [SMILES: COc1cc2cc(c1Cl)N(C)C(=O)C[C@H](OC(=O)[C@H](C)N(C)C(=O)CCSC1CC(=O)N(C[C@H]3CC[C@H](C(=O)ON4C(=O)CCC4=O)CC3)C1=O)[C@]1(C)O[C@H]1[C@H](C)[C@@H]1C[C@@](O)(NC(=O)O1)[C@H](OC)/C=C/C=C(\C)C2]

wherein the diastereomer is characterized by a ¹H NMR substantially as shown in FIG. 31.

In one embodiment, the antibody is conjugated to a compound having the following structure:

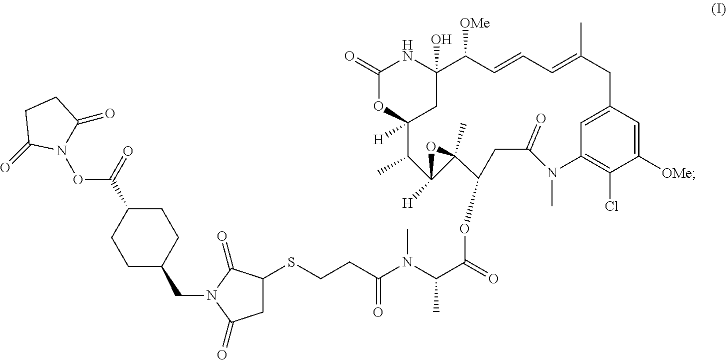 [SMILES: COc1cc2cc(c1Cl)N(C)C(=O)C[C@H](OC(=O)[C@H](C)N(C)C(=O)CCSC1CC(=O)N(C[C@H]3CC[C@H](C(=O)ON4C(=O)CCC4=O)CC3)C1=O)[C@]1(C)O[C@H]1[C@H](C)[C@@H]1C[C@@](O)(NC(=O)O1)[C@H](OC)/C=C/C=C(\C)C2]

prepared by a process comprising the steps of contacting:
(i) a compound of Formula III:

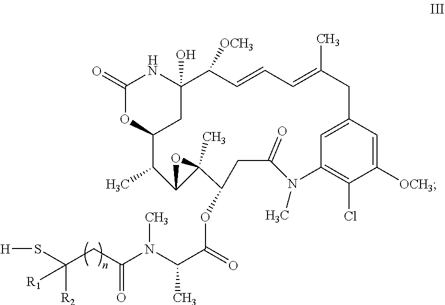

(ii) a compound of formula IV:

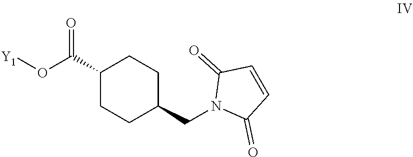

(iii) silica gel; and
(iv) a diluent comprising an organic solvent and water.

In some embodiments, the conjugates have the following structure:

Ab-[L-Pay]ₙ

wherein:
Ab is an anti-MET antibody or a MET×MET bispecific antigen-binding protein as described herein;
L is a linker;
Pay is a cytotoxic agent; and
n is an integer from 1-10.

In some embodiments, Ab is an anti-MET antibody comprising the CDRs within the HCVR/LCVR amino acid sequence pair of SEQ ID NOs: 82/138. In some embodiments, Ab is an anti-MET antibody comprising the HCVR amino acid sequence of SEQ ID NO: 82 and the LCVR amino acid sequence of SEQ ID NO: 138.

In some embodiments, Ab is a MET×MET bispecific antigen-binding protein comprising the CDRs within the D1-HCVR amino acid sequence of SEQ ID NO: 58 and the CDRs within the D2-HCVR amino acid sequence of SEQ ID NO: 82. In some aspects, the MET×MET bispecific antigen-binding protein further comprises the CDRs within the LCVR amino acid sequence of SEQ ID NO: 138. In some embodiments, Ab is a MET×MET bispecific antigen-binding protein comprising the D1-HCVR amino acid sequence of SEQ ID NO: 58 and the D2-HCVR amino acid sequence of SEQ ID NO: 82. In some aspects, the MET×MET bispecific antigen-binding protein further comprises the LCVR amino acid sequence of SEQ ID NO: 138.

In some embodiments, Ab is a MET×MET bispecific antigen-binding protein comprising the CDRs within the D1-HCVR amino acid sequence of SEQ ID NO: 18 and the CDRs within the D2-HCVR amino acid sequence of SEQ ID NO: 82. In some aspects, the MET×MET bispecific antigen-binding protein further comprises the CDRs within the LCVR amino acid sequence of SEQ ID NO: 138. In some embodiments, Ab is a MET×MET bispecific antigen-binding protein comprising the D1-HCVR amino acid sequence of SEQ ID NO: 18 and the D2-HCVR amino acid sequence of SEQ ID NO: 82.

In some embodiments, L is a cleavable linker. In some embodiments, L is a non-cleavable linker. In some embodiments, L comprises a dipeptide. In some embodiments, L comprises a PAB moiety.

In some embodiments, L comprises a moiety having the following structure:

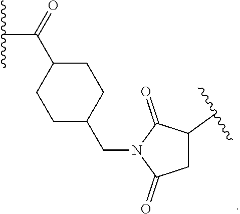 [SMILES: *C(=O)C1CCC(CN2C(=O)CC(*)C2=O)CC1]

In some embodiments, L comprises a moiety having the following structure:

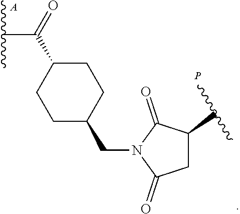

In some embodiments, L comprises a moiety having the following structure:

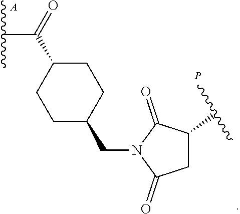

In some embodiments, L comprises a moiety having the following structure:

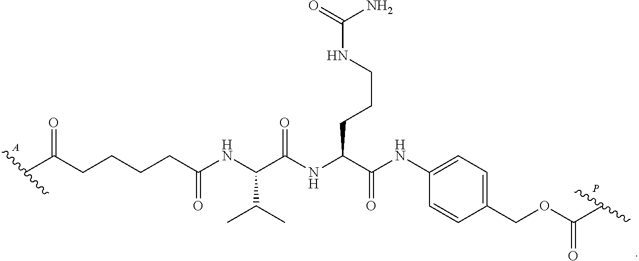

In some embodiments, Pay is a maytansinoid.

In some embodiments, Pay is:

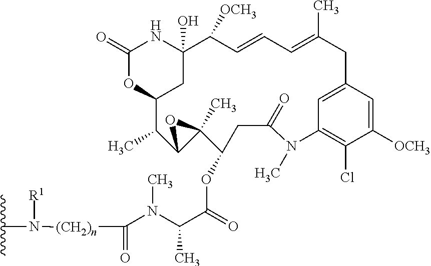

wherein R¹ is alkyl.

In some embodiments, Pay is:

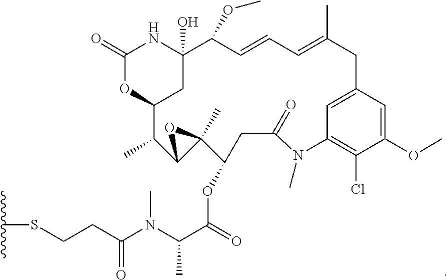 [SMILES: *SCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In some embodiments, Pay is:

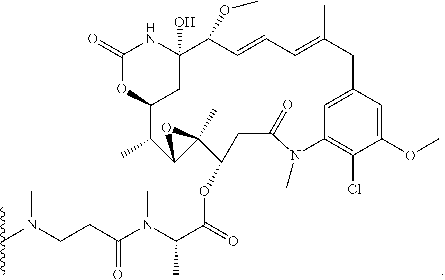 [SMILES: *N(C)CCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In some embodiments, n is an integer from 2 to 5.

In some embodiments, -L-Pay is:

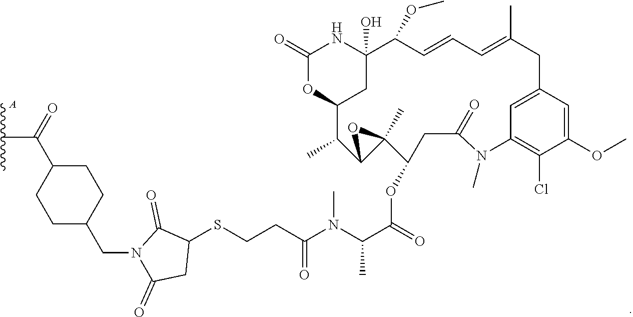

wherein [symbol] is a bond to the antibody.

In some embodiments, -L-Pay is:

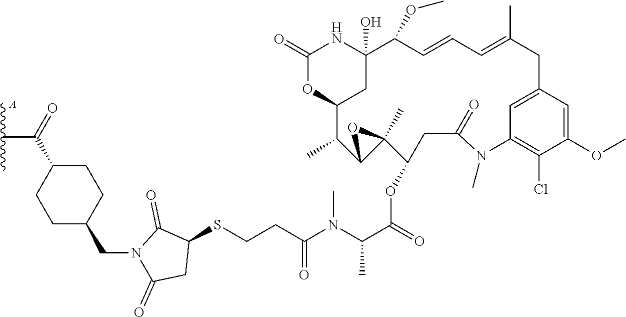

wherein [symbol] is a bond to the antibody.

In some embodiments, -L-Pay is

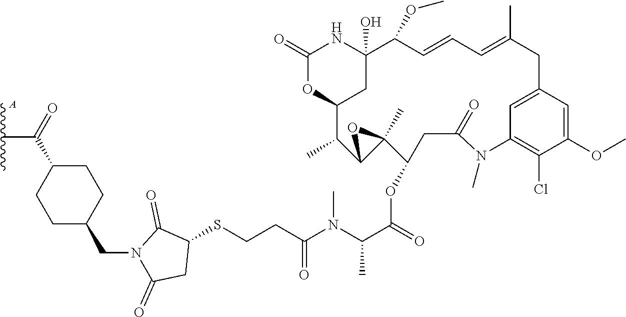

wherein [symbol] is a bond to the antibody.

In some embodiments, -L-Pay is:

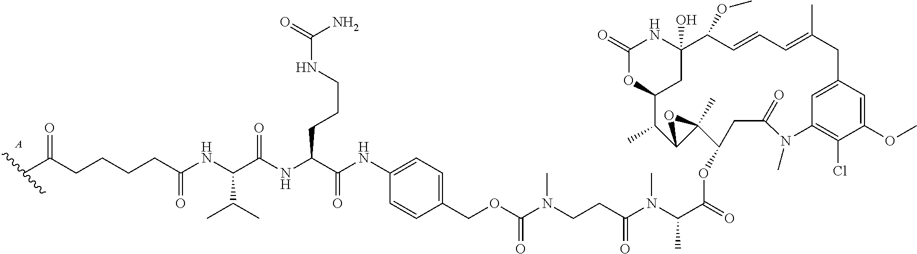

wherein [symbol] is a bond to the antibody.

In some embodiments, the conjugates have the following structure:

Ab-[L-Pay]ₙ

wherein:
Ab is an anti-MET antibody comprising the HCVR amino acid sequence of SEQ ID NO: 82 and the LCVR amino acid sequence of SEQ ID NO: 138;

### L-Pay is

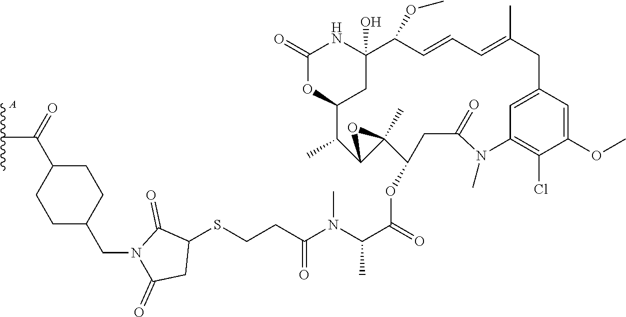

wherein [symbol] is a bond to the antibody; and n is an integer from 2-5.

In some embodiments, the conjugates have the following structure:

Ab-[L-Pay]ₙ

wherein:
Ab is an anti-MET antibody comprising the HCVR amino acid sequence of SEQ ID NO: 82 and the LCVR amino acid sequence of SEQ ID NO: 138;

### L-Pay is

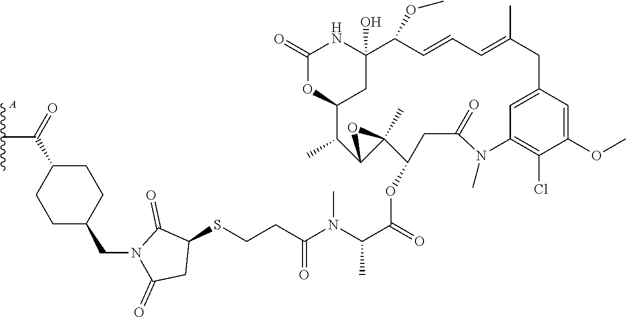

wherein [symbol] is a bond to the antibody; and n is an integer from 2-5.

In some embodiments, the conjugates have the following structure:

Ab-[L-Pay]ₙ

wherein:
Ab is an anti-MET antibody comprising the HCVR amino acid sequence of SEQ ID NO: 82 and the LCVR amino acid sequence of SEQ ID NO: 138;

### L-Pay is

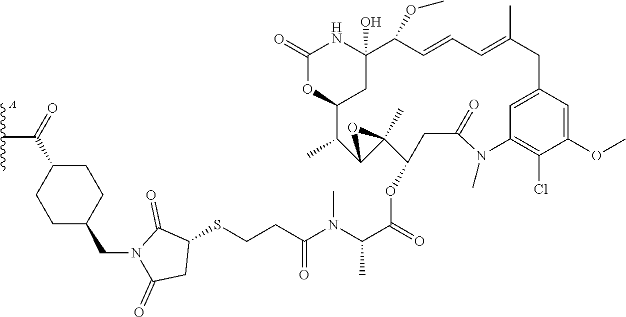

wherein [symbol] is a bond to the antibody; and n is an integer from 2-5.

In some embodiments, the conjugates have the following structure:

Ab-[L-Pay]ₙ

wherein:
Ab is an anti-MET antibody comprising the HCVR amino acid sequence of SEQ ID NO: 82 and the LCVR amino acid sequence of SEQ ID NO: 138;

### L-Pay is

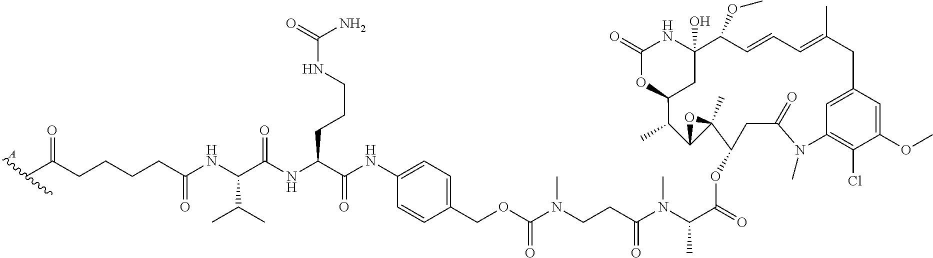

wherein [symbol] is a bond to the antibody; and n is an integer from 2-5.

In some embodiments, the conjugates have the following structure:

Ab-[L-Pay]ₙ

wherein:
Ab is a MET×MET bispecific antigen-binding protein comprising the D1-HCVR amino acid sequence of SEQ ID NO: 58 and the D2-HCVR amino acid sequence of SEQ ID NO: 82;

### L-Pay is

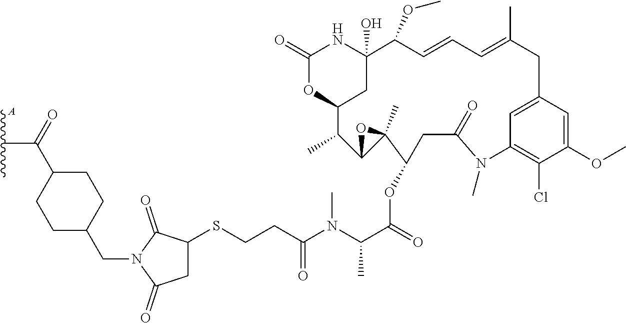

wherein [symbol] is a bond to the antigen binding protein; and n is an integer from 2-5.

In some embodiments, the conjugates have the following structure:

Ab-[L-Pay]ₙ

wherein:
Ab is a MET×MET bispecific antigen-binding protein comprising the D1-HCVR amino acid sequence of SEQ ID NO: 58 and the D2-HCVR amino acid sequence of SEQ ID NO: 82;

### L-Pay is

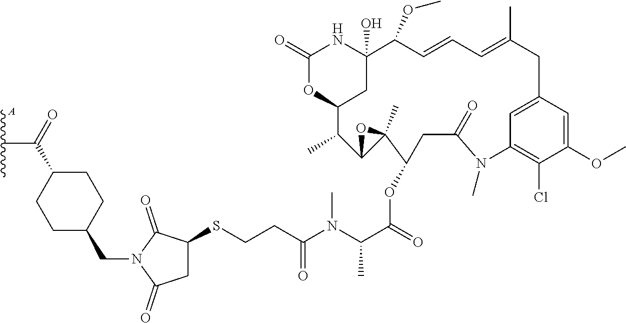

wherein [symbol] is a bond to the antigen-binding protein; and n is an integer from 2-5.

In some embodiments, the conjugates have the following structure:

Ab-[L-Pay]ₙ

wherein:
Ab is a MET×MET bispecific antigen-binding protein comprising the D1-HCVR amino acid sequence of SEQ ID NO: 58 and the D2-HCVR amino acid sequence of SEQ ID NO: 82;

### L-Pay is

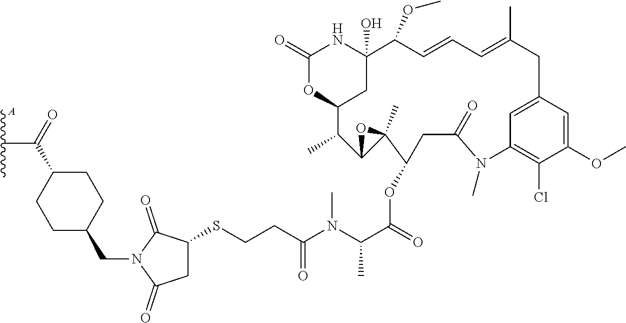

wherein [symbol] is a bond to the antigen-binding protein; and n is an integer from 2-5.

In some embodiments, the conjugates have the following structure:

Ab-[L-Pay]ₙ

wherein:
Ab is a MET×MET bispecific antigen-binding protein comprising the D1-HCVR amino acid sequence of SEQ ID NO: 58 and the D2-HCVR amino acid sequence of SEQ ID NO: 82;

### L-Pay is

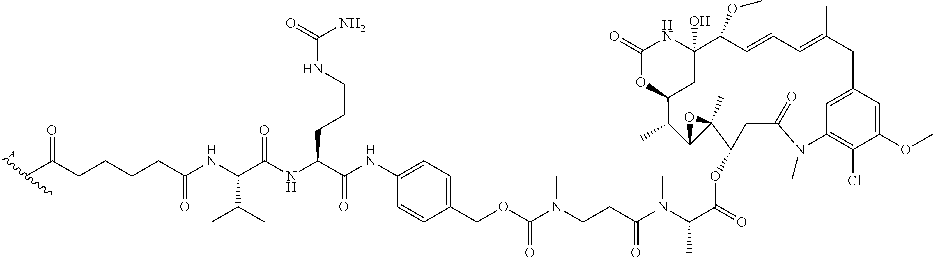

wherein [symbol] is a bond to the antigen-binding protein; and n is an integer from 2-5.

In some embodiments, the conjugates have the following structure:

Ab-[L-Pay]ₙ

wherein:
Ab is a MET×MET bispecific antigen-binding protein comprising the D1-HCVR amino acid sequence of SEQ ID NO: 18 and the D2-HCVR amino acid sequence of SEQ ID NO: 82;

### L-Pay is

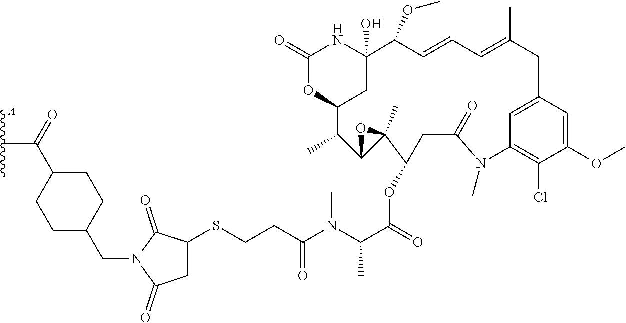

wherein [symbol] is a bond to the antigen-binding protein; and n is an integer from 2-5.

In some embodiments, the conjugates have the following structure:

Ab-[L-Pay]ₙ

wherein:
Ab is a MET×MET bispecific antigen-binding protein comprising the D1-HCVR amino acid sequence of SEQ ID NO: 18 and the D2-HCVR amino acid sequence of SEQ ID NO: 82;

### L-Pay is

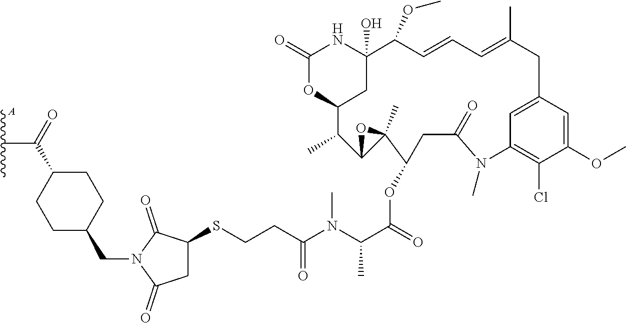

wherein [symbol] is a bond to the antigen-binding protein; and n is an integer from 2-5.

In some embodiments, the conjugates have the following structure:

Ab-[L-Pay]ₙ

wherein:
Ab is a MET×MET bispecific antigen-binding protein comprising the D1-HCVR amino acid sequence of SEQ ID NO: 18 and the D2-HCVR amino acid sequence of SEQ ID NO: 82;

### L-Pay is

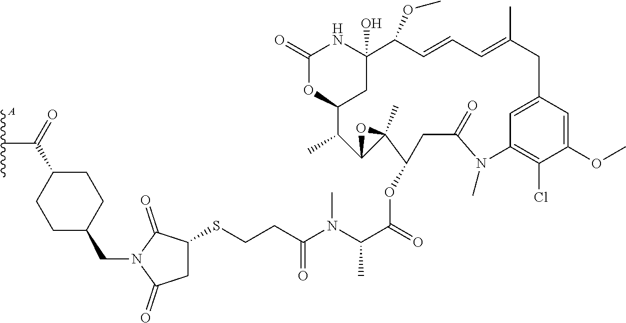

wherein [symbol] is a bond to the antigen-binding protein; and n is an integer from 2-5.

In some embodiments, the conjugates have the following structure:

Ab-[L-Pay]ₙ

wherein:
Ab is a MET×MET bispecific antigen-binding protein comprising the D1-HCVR amino acid sequence of SEQ ID NO: 18 and the D2-HCVR amino acid sequence of SEQ ID NO: 82;

### L-Pay is

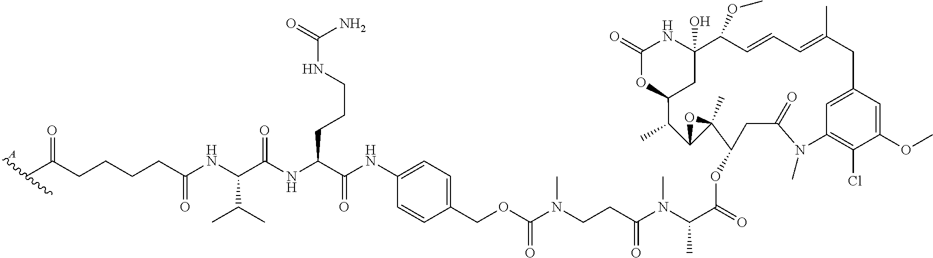

wherein [symbol] is a bond to the antigen-binding protein; and n is an integer from 2-5.

The antibody drug conjugates useful herein can be prepared using conjugation conditions known to those of ordinary skill in the art, (see, e.g., Doronina et al. Nature Biotechnology 2003, 21, 7, 778, which is incorporated herein by reference in its entirety). In some embodiments an anti-MET antibody or a MET×MET bispecific antigen-binding protein antibody drug conjugate is prepared by contacting an anti-MET antibody or a MET×MET bispecific antigen-binding protein described herein with a compound comprising the desired linker and cytotoxic agent, wherein said linker possesses a moiety that is reactive with the antibody or antigen-binding protein, e.g., at the desired residue of the antibody or antigen-binding protein.

In some embodiments, processes for preparing an antibody-drug conjugate useful according to the methods provided herein comprise contacting an anti-MET antibody or a MET×MET bispecific antigen-binding protein described herein with a compound having the following formula A¹:

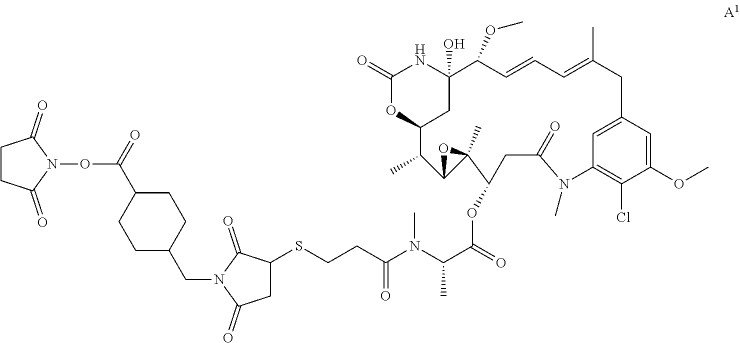 [SMILES: COc1cc2cc(c1Cl)N(C)C(=O)C[C@H](OC(=O)[C@H](C)N(C)C(=O)CCSC1CC(=O)N(CC3CCC(C(=O)ON4C(=O)CCC4=O)CC3)C1=O)[C@]1(C)O[C@H]1[C@H](C)[C@@H]1C[C@@](O)(NC(=O)O1)[C@H](OC)/C=C/C=C(\C)C2]

and aqueous diluent.

In some embodiments, the compound of formula A¹ is present in stoichiometric excess. In some embodiments, the compound of formula A¹ is present in 5-6 fold stoichiometric excess. In some embodiments, the aqueous diluent comprises HEPES. In some embodiments, the aqueous diluent comprises DMA.

In some embodiments, the compound of formula A¹ is a compound of formula A² or A³:

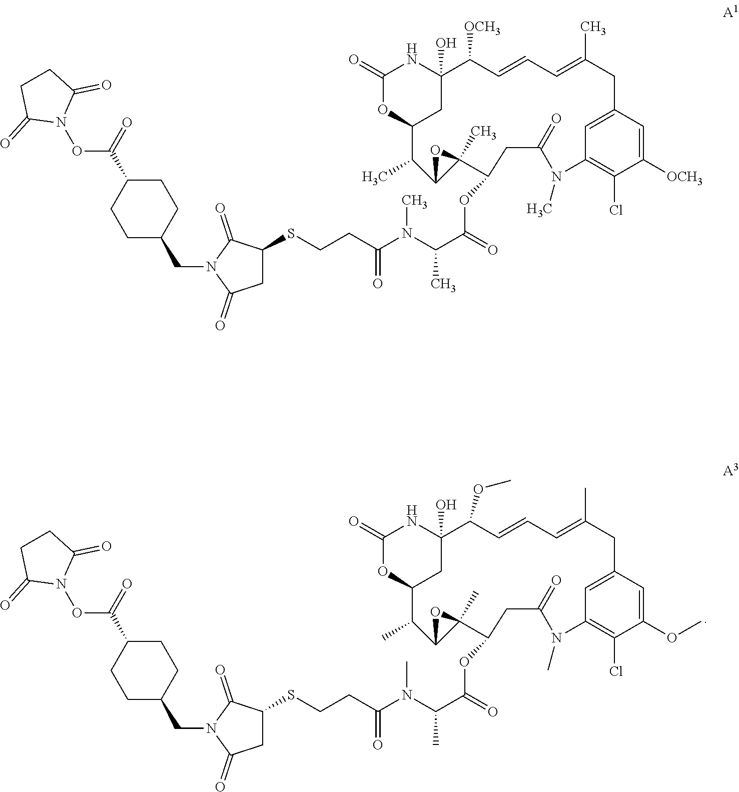 [SMILES: COc1cc2cc(c1Cl)N(C)C(=O)C[C@H](OC(=O)[C@H](C)N(C)C(=O)CCS[C@H]1CC(=O)N(C[C@H]3CC[C@H](C(=O)ON4C(=O)CCC4=O)CC3)C1=O)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)CCS[C@@H]1CC(=O)N(C[C@H]3CC[C@H](C(=O)ON4C(=O)CCC4=O)CC3)C1=O)[C@]1(C)O[C@H]1[C@H](C)[C@@H]1C[C@@](O)(NC(=O)O1)[C@H](OC)/C=C/C=C(\C)C2]

In some embodiments, the compound of formula A² or A³ is stereometrically pure. In some embodiments, the compound of formula A¹ comprises a compound of formula A² or A³, wherein the compound of A² or A³ is present in a diastereomeric excess of more than 50%. In certain embodiments, the diastereomeric excess is more than 70%. In certain embodiments, the diastereomeric excess is more than 90%. In certain embodiments, the diastereomeric excess is more than 95%.

The term “diastereomeric excess” refers to the difference between the mole fraction of the desired single diastereomer as compared to the remaining diastereomers in a composition. Diastereomeric excess is calculated as follows: (amount of single diastereomer)−(amount of other diastereomers)/1. For example, a composition that contains 90% of 1 and 10% of 2, 3, 4, or a mixture thereof has a diastereomeric excess of 80% [(90−10)/1]. A composition that contains 95% of 1 and 5% of 2, 3, 4, or a mixture thereof has a diastereomeric excess of 90% [(95−5)/1]. A composition that contains 99% of 1 and 1% of 2, 3, 4, or a mixture thereof has a diastereomeric excess of 98% [(99−1)/1]. The diastereomeric excess can similarly be calculated for any one of 1, 2, 3, or 4.

In some embodiments, the compound of formula A¹ is prepared by contacting a compound of formula (a):

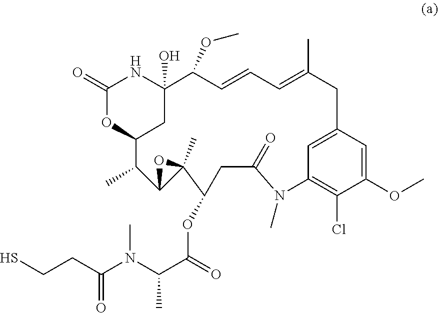 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2]

with a compound of formula (b)

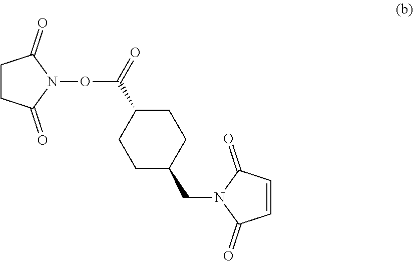 [SMILES: O=C1C=CC(=O)N1C[C@H]1CC[C@H](C(=O)ON2C(=O)CCC2=O)CC1]

in the presence of silica gel and diluent. In some embodiments, the diluent comprises an organic solvent and water.

Provided herein is also the product prepared by the process of:

(i) contacting a compound of formula (a):

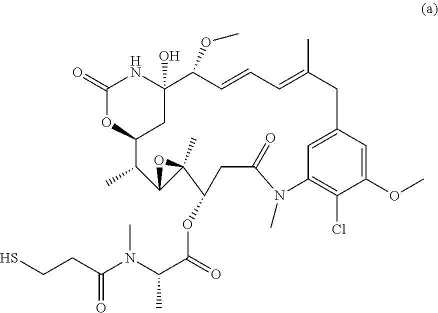 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2]

with a compound of formula (b):

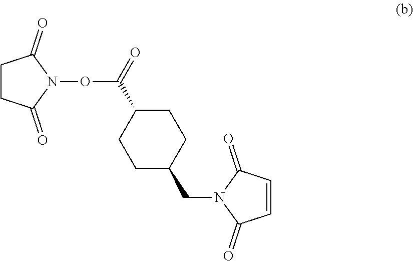 [SMILES: O=C1C=CC(=O)N1C[C@H]1CC[C@H](C(=O)ON2C(=O)CCC2=O)CC1]

in the presence of silica gel and diluent to synthesize an intermediate; and

(ii) contacting an anti-MET antibody or a MET×MET bispecific antigen-binding protein described herein with the intermediate and aqueous diluent.

In some embodiments, provided herein are processes for preparing an antibody-drug conjugate comprising contacting an anti-MET antibody or a MET×MET bispecific antigen-binding protein described herein with a compound having the following formula B:

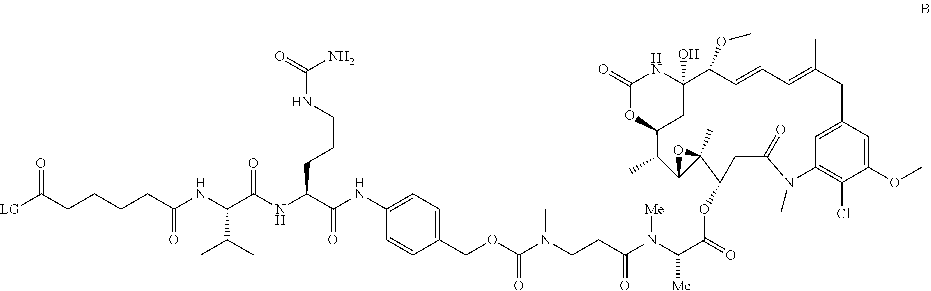

wherein LG is a leaving group, and aqueous diluent.

In some embodiments, the compound of formula B is present in stoichiometric excess. In some embodiments, the compound of formula B is present in 5-6 fold stoichiometric excess. In some embodiments, the aqueous diluent comprises HEPES. In some embodiments, the aqueous diluent comprises DMA. In some embodiments, the —C(O)-LG is an ester, e.g., NHS or trifluorophenyl ester.

In some embodiments, the compound of formula B is a compound of formula B¹:

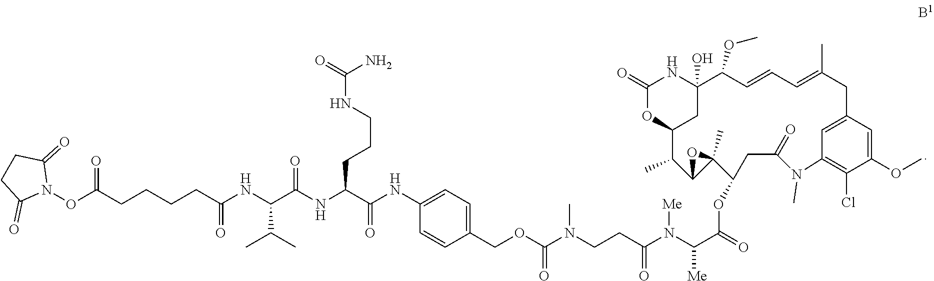 [SMILES: COc1cc2cc(c1Cl)N(C)C(=O)C[C@H](OC(=O)[C@H](C)N(C)C(=O)CCN(C)C(=O)OCc1ccc(NC(=O)[C@H](CCCNC(N)=O)NC(=O)[C@@H](NC(=O)CCCCC(=O)ON3C(=O)CCC3=O)C(C)C)cc1)[C@]1(C)O[C@H]1[C@H](C)[C@@H]1C[C@@](O)(NC(=O)O1)[C@H](OC)/C=C/C=C(\C)C2]

In some embodiments, the compound of formula B¹ is prepared by contacting a compound of formula C:

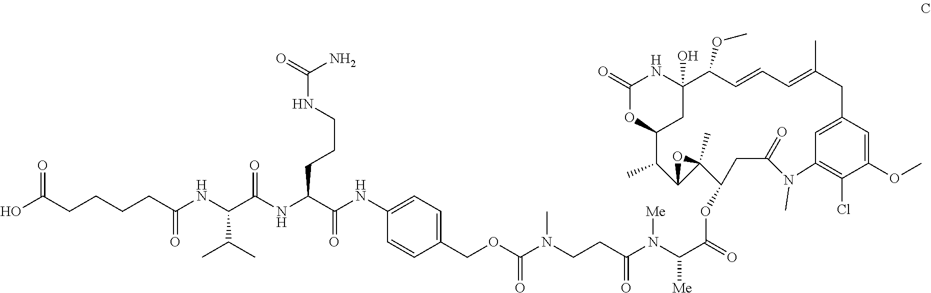 [SMILES: COc1cc2cc(c1Cl)N(C)C(=O)C[C@H](OC(=O)[C@H](C)N(C)C(=O)CCN(C)C(=O)OCc1ccc(NC(=O)[C@H](CCCNC(N)=O)NC(=O)[C@@H](NC(=O)CCCCC(=O)O)C(C)C)cc1)[C@]1(C)O[C@H]1[C@H](C)[C@@H]1C[C@@](O)(NC(=O)O1)[C@H](OC)/C=C/C=C(\C)C2]

with N-hydroxysuccinimide (NHS), a peptide coupling reagent, and an organic diluent. Suitable peptide coupling reagents include those that activate, i.e., render reactive, carboxylic acid moieties for reaction with a nucleophile. In certain embodiments, the peptide coupling reagent is N-(3-dimethylaminopropyl)-N′-ethylcarbodiimide hydrochloride (EDC). In some embodiments, the organic solvent is dichloromethane.

In some embodiments, the compound of formula C is prepared by contacting a compound of formula D:

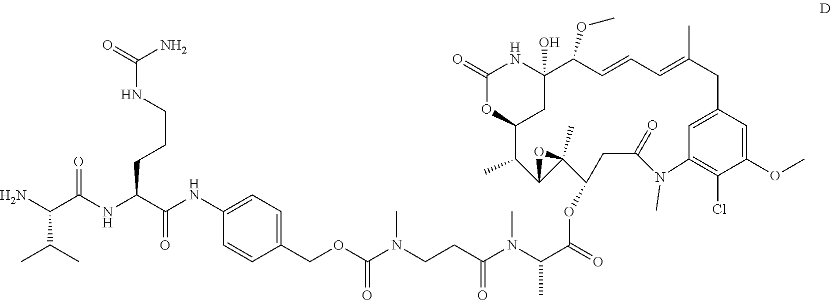 [SMILES: COc1cc2cc(c1Cl)N(C)C(=O)C[C@H](OC(=O)[C@H](C)N(C)C(=O)CCN(C)C(=O)OCc1ccc(NC(=O)[C@H](CCCNC(N)=O)NC(=O)[C@@H](N)C(C)C)cc1)[C@]1(C)O[C@H]1[C@H](C)[C@@H]1C[C@@](O)(NC(=O)O1)[C@H](OC)/C=C/C=C(\C)C2]

with adipic acid, a peptide coupling agent, and an organic solvent. In certain embodiments, the peptide coupling agent is 2-ethoxy-1-ethoxycarbonyl-1,2-dihydroquinoline (EEDQ). In certain embodiments, the organic solvent comprises dichloromethane. Compound D can be prepared as described in WO2014/145090.

### Epitope Mapping and Related Technologies

The epitope to which the antibodies and antigen-binding domains bind may consist of a single contiguous sequence of 3 or more (e.g., 3, 4, 5, 6, 7, 8, 9, 10, 11, 12, 13, 14, 15, 16, 17, 18, 19, 20 or more) amino acids of a MET protein. Alternatively, the relevant epitope may consist of a plurality of non-contiguous amino acids (or amino acid sequences) of MET. In some embodiments, the epitope is located on or near the ligand-binding domain of MET. In other embodiments, the epitope is located outside of the ligand-binding domain of MET, e.g., at a location on the surface of MET at which an antibody, when bound to such an epitope, does not interfere with HGF binding to MET.

As described elsewhere herein, the individual antigen binding domains (D1 and D2) of the MET×MET bispecific antigen-binding molecules may bind to distinct, or non-overlapping, or partially overlapping epitopes, relative to one another. As used herein, “partially overlapping epitopes” means that the first and second epitopes share less than 5, less than 4, less than 3, or only one common amino acid as determined by any epitope mapping methodology known in the art (e.g., X-ray crystallography, alanine-scan mutagenesis, hydrogen/deuterium exchange [HDX], domain swapping, etc.). The D1 and D2 domains may be non-competitive with one another. For example, in certain embodiments, the binding of a D1 domain of a particular MET×MET bispecific antigen-binding molecule to its epitope on MET does not inhibit (or only minimally inhibits) the binding of the D2 domain of the MET×MET bispecific antigen-binding molecule to its epitope on MET. Due to the non-overlapping (or at most, partially overlapping) nature of the respective epitopes of the D1 and D2 components, the MET×MET bispecific antigen-binding molecules are able to bind to a single MET molecule on a cell surface.

Various techniques known to persons of ordinary skill in the art can be used to determine the epitope on MET with which the antibodies and antigen-binding domains useful herein interact. Exemplary techniques that can be used to determine an epitope or binding domain of a particular antibody or antigen-binding domain include, e.g., point mutagenesis (e.g., alanine scanning mutagenesis, arginine scanning mutagenesis, etc.), peptide blots analysis (Reineke, 2004, Methods Mol Biol 248:443-463), protease protection, and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A. X-ray crystal structure analysis can also be used to identify the amino acids within a polypeptide with which an antibody interacts.

Useful according to the methods provided herein are anti-MET antibodies (including bispecific antibodies) that bind to the same epitope as any of the specific exemplary antibodies or antigen-binding domains described herein (e.g. antibodies comprising any of the amino acid sequences as set forth in Table 1 herein). Likewise, also provided herein are anti-MET antibodies that compete for binding to MET with any of the specific exemplary antibodies described herein (e.g. antibodies comprising any of the amino acid sequences as set forth in Table 1 herein). In some embodiments, the human MET epitope to which the anti-MET antibodies bind comprises amino acids 192-204, amino acids 305-315, and/or amino acids 421-455 of SEQ ID NO:155. In some embodiments, the first epitope of human MET comprises amino acids 192-204 of SEQ ID NO:155; and the second epitope of human MET comprises amino acids 305-315 and 421-455 of SEQ ID NO:155.

One can easily determine whether an antibody binds to the same epitope as, or competes for binding with, a reference anti-MET antibody by using routine methods known in the art and exemplified herein. For example, to determine if a test antibody binds to the same epitope as a reference anti-MET antibody provided herein, the reference antibody is allowed to bind to a MET protein. Next, the ability of a test antibody to bind to the MET molecule is assessed. If the test antibody is able to bind to MET following saturation binding with the reference anti-MET antibody, it can be concluded that the test antibody binds to a different epitope than the reference anti-MET antibody. On the other hand, if the test antibody is not able to bind to the MET molecule following saturation binding with the reference anti-MET antibody, then the test antibody may bind to the same epitope as the epitope bound by the reference anti-MET antibody. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990:50:1495-1502). Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if an antibody competes for binding (or cross-competes for binding) with a reference anti-MET antibody, the above-described binding methodology is performed in two orientations: In a first orientation, the reference antibody is allowed to bind to a MET protein under saturating conditions followed by assessment of binding of the test antibody to the MET molecule. In a second orientation, the test antibody is allowed to bind to a MET molecule under saturating conditions followed by assessment of binding of the reference antibody to the MET molecule. If, in both orientations, only the first (saturating) antibody is capable of binding to the MET molecule, then it is concluded that the test antibody and the reference antibody compete for binding to MET. As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

### Preparation of Human Antibodies

The anti-MET antibodies and MET×MET bispecific antibodies useful according to the methods provided herein can be fully human antibodies. Methods for generating monoclonal antibodies, including fully human monoclonal antibodies are known in the art. Any such known methods can be used in the context of the present disclosure to make human antibodies that specifically bind to human MET.

Using VELOCIMMUNE™ technology, for example, or any other similar known method for generating fully human monoclonal antibodies, high affinity chimeric antibodies to MET are initially isolated having a human variable region and a mouse constant region. As in the experimental section below, the antibodies are characterized and selected for desirable characteristics, including affinity, ligand blocking activity, selectivity, epitope, etc. If necessary, mouse constant regions are replaced with a desired human constant region, for example wild-type or modified IgG1 or IgG4, to generate a fully human anti-MET antibody. While the constant region selected may vary according to specific use, high affinity antigen-binding and target specificity characteristics reside in the variable region. In certain instances, fully human anti-MET antibodies are isolated directly from antigen-positive B cells.

### Bioequivalents

The anti-MET antibodies and antibody fragments useful according to the methods provided herein encompass proteins having amino acid sequences that vary from those of the described antibodies but that retain the ability to bind human MET. Such variant antibodies and antibody fragments comprise one or more additions, deletions, or substitutions of amino acids when compared to parent sequence, but exhibit biological activity that is essentially equivalent to that of the described antibodies. Likewise, the anti-MET antibody-encoding DNA sequences of the present disclosure encompass sequences that comprise one or more additions, deletions, or substitutions of nucleotides when compared to the disclosed sequence, but that encode an anti-MET antibody or antibody fragment that is essentially bioequivalent to an anti-MET antibody or antibody fragment of the disclosure. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-MET antibodies provided herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include anti-MET antibody variants comprising amino acid changes which modify the glycosylation characteristics of the antibodies, e.g., mutations which eliminate or remove glycosylation.

### Species Selectivity and Species Cross-Reactivity

The present disclosure, according to certain embodiments, provides anti-MET antibodies (and antigen-binding molecules comprising anti-MET antigen-binding domains) that bind to human MET but not to MET from other species, and are useful in treating eye cancers such as uveal melanoma, orbital lymphoma, retinoblastoma, and medulloepithelioma. The present disclosure also includes anti-MET antibodies (and antigen-binding molecules comprising anti-MET antigen-binding domains) that bind to human MET and to MET from one or more non-human species, and are useful in treating eye cancers such as uveal melanoma, orbital lymphoma, retinoblastoma, and medulloepithelioma. For example, the anti-MET antibodies and antigen-binding molecules may bind to human MET and may bind or not bind, as the case may be, to one or more of mouse, rat, guinea pig, hamster, gerbil, pig, cat, dog, rabbit, goat, sheep, cow, horse, camel, cynomologous, marmoset, rhesus or chimpanzee MET. According to certain exemplary embodiments, anti-MET antibodies and antigen-binding molecules are provided which specifically bind human MET and cynomolgus monkey (e.g., Macaca fascicularis) MET. Other anti-MET antibodies and antigen-binding molecules bind human MET but do not bind, or bind only weakly, to cynomolgus monkey MET.

### Multispecific Antibodies

As described elsewhere herein, useful according to the present disclosure are bispecific antigen-binding molecules comprising two different antigen-binding domains, wherein the first antigen-binding domain (D1) binds a first epitope on MET, and wherein the second antigen-binding domain (D2) binds a second epitope on MET. In certain embodiments, the first and second epitopes on MET to which the D1 and D2 domains bind are distinct, or non-overlapping, or partially overlapping. According to this aspect, the D1 domain can comprise any of the HCVR/LCVR or CDR amino acid sequences as set forth in Table 1 herein, and the D2 domain can comprise any other of the HCVR/LCVR or CDR amino acid sequences as set forth in Table 1 herein (so long as the binding specificity of the D1 domain is different from the binding specificity of the D2 domain, and/or the antigen-binding protein from which D1 was obtained does not compete for binding to MET with the antigen-binding protein from which D2 was obtained). In some embodiments, the human MET epitope to which the anti-MET antibodies bind comprises amino acids 192-204, amino acids 305-315, and/or amino acids 421-455 of SEQ ID NO:155. In some embodiments, the first epitope of human MET comprises amino acids 192-204 of SEQ ID NO:155; and the second epitope of human MET comprises amino acids 305-315 and 421-455 of SEQ ID NO:155.

According to a separate aspect, conventional bispecific antibodies are also provided as useful herein wherein one arm of the bispecific antibody binds to an epitope on human MET, and the other arm of the bispecific antibody binds to a second antigen other than MET. The MET-binding arm can comprise any of the HCVR/LCVR or CDR amino acid sequences as set forth in Table 1 herein. In certain embodiments, the MET-binding arm binds human MET and blocks HGF binding to MET. In other embodiments, the MET-binding arm binds human MET but does not block HGF binding to MET.

An exemplary bispecific antibody format that can be used in the context of the present disclosure involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bispecific antibody format described above are contemplated within the scope of the present disclosure.

Other exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab^{e} bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

### Therapeutic Formulation and Administration

Provided herein are pharmaceutical compositions comprising the anti-MET antibodies or MET×MET bispecific antigen-binding molecules useful according to the methods described herein. The pharmaceutical compositions may be formulated with suitable carriers, excipients, and other agents that provide improved transfer, delivery, tolerance, and the like.

In some aspects, the pharmaceutical compositions comprising the anti-MET antibodies or MET×MET bispecific antigen-binding molecules are formulated for administration to the eye to treat eye cancer such as uveal melanoma, orbital lymphoma, retinoblastoma, or medulloepithelioma.

Provided herein are methods in which the anti-MET antibodies or the MET×MET bispecific antigen-binding molecules that are administered to the patient are contained within a pharmaceutical formulation. The pharmaceutical formulation may comprise the anti-MET antibody or MET×MET bispecific antigen-binding molecule along with at least one inactive ingredient such as, e.g., a pharmaceutically acceptable carrier. Other agents may be incorporated into the pharmaceutical composition to provide improved transfer, delivery, tolerance, and the like. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the antibody is administered. A multitude of appropriate formulations can be found in the formulary known to all pharmaceutical chemists: Remington's Pharmaceutical Sciences (15th ed, Mack Publishing Company, Easton, Pa., 1975), particularly Chapter 87 by Blaug, Seymour, therein. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in the context of the methods of the present disclosure, provided that the anti-MET antibody or MET×MET bispecific antigen-binding molecule is not inactivated by the formulation and the formulation is physiologically compatible and tolerable with the route of administration. See also Powell et al. PDA (1998) J Pharm Sci Technol. 52:238-311 and the citations therein for additional information related to excipients and carriers well known to pharmaceutical chemists.

Pharmaceutical formulations useful for administration by injection in the context of the present disclosure may be prepared by dissolving, suspending or emulsifying an anti-MET antibody or MET×MET bispecific antigen-binding molecule in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there may be employed, e.g., sesame oil, soybean oil, etc., which may be used in combination with a solubilizing agent such as benzyl benzoate, benzyl alcohol, etc. The injection thus prepared can be filled in an appropriate ampoule if desired.

### Modes of Administration

The anti-MET antibodies and MET×MET bispecific antigen-binding molecules (or pharmaceutical formulation comprising the anti-MET antibodies and MET×MET bispecific antigen-binding molecules) may be administered to the patient by any known delivery system and/or administration method. In certain embodiments, the anti-MET antibodies and MET×MET bispecific antigen-binding molecules are administered to the patient by ocular, intraocular, intravitreal or subconjunctival injection. In other embodiments, the anti-MET antibodies and MET×MET bispecific antigen-binding molecules can be administered to the patient by topical administration, e.g., via eye drops or other liquid, gel, ointment or fluid which contains the anti-MET antibodies and MET×MET bispecific antigen-binding molecules and can be applied directly to the eye. Other possible routes of administration include, e.g., intradermal, intramuscular, intraperitoneal, intravenous, subcutaneous, intranasal, epidural, and oral.

### Combination Therapies and Formulations

Provided herein are compositions and therapeutic formulations comprising any of the anti-MET antibodies and MET×MET bispecific antigen-binding molecules described herein in combination with one or more additional therapeutically active components, and methods of treatment comprising administering such combinations to subjects in need thereof.

The anti-MET antibodies and MET×MET bispecific antigen-binding molecules may be co-formulated with and/or administered in combination with one or more additional therapeutically active component(s) selected from the group consisting of: a MET antagonist (e.g., an anti-MET antibody [e.g., onartuzumab, emibetuzumab, telisotuzumab, SAIT301, ARGX-111, Sym015, HuMax-cMet, CE-355621, and H4H14639D] or small molecule inhibitor of MET), an EGFR antagonist (e.g., an anti-EGFR antibody [e.g., cetuximab or panitumumab] or small molecule inhibitor of EGFR [e.g., gefitinib or erlotinib]), an antagonist of another EGFR family member such as Her2/ErbB2, ErbB3 or ErbB4 (e.g., anti-ErbB2 [e.g., trastuzumab or T-DM1 {KADCYLA®}], anti-ErbB3 or anti-ErbB4 antibody or small molecule inhibitor of ErbB2, ErbB3 or ErbB4 activity), an antagonist of EGFRvIII (e.g., an anti-EGFRvIII antibody), an IGF1R antagonist (e.g., an anti-IGF1R antibody), a B-raf inhibitor (e.g., vemurafenib, sorafenib, GDC-0879, PLX-4720), a PDGFR-α inhibitor (e.g., an anti-PDGFR-α antibody), a PDGFR-β inhibitor (e.g., an anti-PDGFR-β antibody or small molecule kinase inhibitor such as, e.g., imatinib mesylate or sunitinib malate), a PDGF ligand inhibitor (e.g., anti-PDGF-A, —B, —C, or -D antibody, aptamer, siRNA, etc.), a VEGF antagonist (e.g., a VEGF-Trap such as aflibercept, see, e.g., U.S. Pat. No. 7,087,411 (also referred to herein as a “VEGF-inhibiting fusion protein”), anti-VEGF antibody (e.g., bevacizumab), a small molecule kinase inhibitor of VEGF receptor (e.g., sunitinib, sorafenib or pazopanib)), a DLL4 antagonist (e.g., an anti-DLL4 antibody disclosed in US 2009/0142354 such as REGN421), an Ang2 antagonist (e.g., an anti-Ang2 antibody disclosed in US 2011/0027286 such as H1H685P), a FOLH1 antagonist (e.g., an anti-FOLH1 antibody), a STEAP1 or STEAP2 antagonist (e.g., an anti-STEAP1 antibody or an anti-STEAP2 antibody), a TMPRSS2 antagonist (e.g., an anti-TMPRSS2 antibody), a MSLN antagonist (e.g., an anti-MSLN antibody), a CA9 antagonist (e.g., an anti-CA9 antibody), a uroplakin antagonist (e.g., an anti-uroplakin [e.g., anti-UPK3A] antibody), a MUC16 antagonist (e.g., an anti-MUC16 antibody), a Tn antigen antagonist (e.g., an anti-Tn antibody), a CLEC12A antagonist (e.g., an anti-CLEC12A antibody), a TNFRSF17 antagonist (e.g., an anti-TNFRSF17 antibody), a LGR5 antagonist (e.g., an anti-LGR5 antibody), a monovalent CD20 antagonist (e.g., a monovalent anti-CD20 antibody such as rituximab), a CD20×CD3 bispecific antibody, a PD-1 blocking agent (e.g., an anti-PD-1 antibody such as pembrolizumab or nivolumab), etc. Other agents that may be beneficially administered in combination with antibodies provided herein include, e.g., tamoxifen, aromatase inhibitors, and cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors.

Illustratively, a PD-1 inhibitor such as an anti-PD-1 antibody can be combined with an anti-Met antibody-drug conjugate as described herein. The target patient population includes specifically those patients with tumors that overexpress the c-Met mutation, such as a patient with a c-Met-expressing uveal melanoma or a c-Met-expressing non-small cell lung cancer.

Provided herein are compositions and therapeutic formulations comprising any of the anti-MET antibodies and MET×MET bispecific antigen-binding molecules described herein in combination with one or more chemotherapeutic agents. Examples of chemotherapeutic agents include alkylating agents such as thiotepa and cyclosphosphamide (Cytoxan™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (Taxol™, Bristol-Myers Squibb Oncology, Princeton, N.J.) and docetaxel (Taxotere™; Aventis Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

The anti-MET antibodies and MET×MET bispecific antigen-binding molecules may also be administered and/or co-formulated in combination with antivirals, antibiotics, analgesics, corticosteroids, steroids, oxygen, antioxidants, COX inhibitors, cardioprotectants, metal chelators, IFN-gamma, and/or NSAIDs.

The additional therapeutically active component(s), e.g., any of the agents listed above or derivatives thereof, may be administered just prior to, concurrent with, or shortly after the administration of an anti-MET antibody or MET×MET bispecific antigen-binding molecule; (for purposes of the present disclosure, such administration regimens are considered the administration of an antibody “in combination with” an additional therapeutically active component). The present disclosure includes pharmaceutical compositions in which an anti-MET antibody or MET×MET bispecific antigen-binding molecule is co-formulated with one or more of the additional therapeutically active component(s) as described elsewhere herein.

### Administration Regimens

According to certain embodiments, multiple doses of an anti-MET antibody or MET×MET bispecific antigen-binding molecule (or a pharmaceutical composition comprising a combination of an anti-MET antibody or MET×MET bispecific antigen-binding molecule and any of the additional therapeutically active agents mentioned herein) may be administered to a subject over a defined time course. The methods according to this aspect comprise sequentially administering to a subject multiple doses of an anti-MET antibody or MET×MET bispecific antigen-binding molecule provided herein. As used herein, “sequentially administering” means that each dose of antibody is administered to the subject at a different point in time, e.g., on different days separated by a predetermined interval (e.g., hours, days, weeks or months). The present disclosure includes methods which comprise sequentially administering to the patient a single initial dose of an anti-MET antibody or MET×MET bispecific antigen-binding molecule, followed by one or more secondary doses of the anti-MET antibody or MET×MET bispecific antigen-binding molecule, and optionally followed by one or more tertiary doses of the anti-MET antibody or MET×MET bispecific antigen-binding molecule.

The terms “initial dose,” “secondary doses,” and “tertiary doses,” refer to the temporal sequence of administration of the anti-MET antibody or MET×MET bispecific antigen-binding molecule. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-MET antibody or MET×MET bispecific antigen-binding molecule, but generally may differ from one another in terms of frequency of administration. In certain embodiments, however, the amount of antibody contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

### Diagnostic Uses of the Antibodies

The anti-MET antibody or MET×MET bispecific antigen-binding molecule of the present disclosure may also be used to detect and/or measure MET, or MET-expressing cells in a sample, e.g., for diagnostic purposes. For example, an anti-MET antibody, or fragment thereof, may be used to diagnose a condition or disease characterized by aberrant expression (e.g., over-expression, under-expression, lack of expression, etc.) of MET. Exemplary diagnostic assays for MET may comprise, e.g., contacting a sample, obtained from a patient, with an anti-MET antibody or MET×MET bispecific antigen-binding molecule, wherein the antibody is labeled with a detectable label or reporter molecule. Alternatively, an unlabeled anti-MET antibody or MET×MET bispecific antigen-binding molecule can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein, or rhodamine; or an enzyme such as alkaline phosphatase, beta-galactosidase, horseradish peroxidase, or luciferase. Specific exemplary assays that can be used to detect or measure MET in a sample include enzyme-linked immunosorbent assay (ELISA), radioimmunoassay (RIA), immuno-PET (e.g., ⁸⁹Zr, ⁶⁴Cu, etc.), and fluorescence-activated cell sorting (FACS).

Samples that can be used in MET diagnostic assays according to the present disclosure include any tissue or fluid sample obtainable from a patient, particularly tissue or fluid found in the eye or ocular cavity. Generally, levels of MET in a particular sample obtained from a healthy patient (e.g., a patient not afflicted with a disease or condition associated with abnormal MET levels or activity) will be measured to initially establish a baseline, or standard, level of MET. This baseline level of MET can then be compared against the levels of MET measured in samples obtained from individuals suspected of having a MET-related disease or condition.

### EXAMPLES

The following examples are put forth so as to provide those of ordinary skill in the art with a complete disclosure and description of how to make and use the methods and compositions provided herein, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Generation of Anti-MET Antibodies

Anti-MET antibodies were obtained by immunizing a genetically engineered mouse comprising DNA encoding human immunoglobulin heavy and kappa light chain variable regions with an immunogen comprising recombinant human MET extracellular domain fused to human Fc (R&D Systems, Catalog #358-MT, Minneapolis, Minn.). The mice used for the immunizations express a “universal light chain.” That is, the antibodies produced in this mouse have different heavy chain variable regions but essentially identical light chain variable domains.

The antibody immune response was monitored by a MET-specific immunoassay. When a desired immune response was achieved splenocytes were harvested and fused with mouse myeloma cells to preserve their viability and form hybridoma cell lines. The hybridoma cell lines were screened and selected to identify cell lines that produce MET-specific antibodies. Using this technique several anti-MET chimeric antibodies (i.e., antibodies possessing human variable domains and mouse constant domains) were obtained. In addition, several fully human anti-MET antibodies were isolated directly from antigen-positive B cells without fusion to myeloma cells, as described in US 2007/0280945A1.

Certain biological properties of the exemplary anti-MET antibodies generated in accordance with the methods of this Example, and bispecific antibodies constructed therefrom, are described in detail in the Examples set forth below.

### Example 2. Heavy and Light Chain Variable Region Amino Acid and Nucleic Acid Sequences

Table 1 sets forth the amino acid sequence identifiers of the heavy and light chain variable regions and CDRs of selected anti-MET antibodies described herein. (As noted above, all antibodies generated in Example 1 possess the same light chain variable region, and thus the same light chain CDR sequences as well). The corresponding nucleic acid sequence identifiers are set forth in Table 2.

TABLE 1
Amino Acid Sequence Identifiers

| | SEQ ID NOs: | | | | | | | |
|---|---|---|---|---|---|---|---|---|
| Antibody Designation | HCVR | HCDR1 | HCDR2 | HCDR3 | LCVR | LCDR1 | LCDR2 | LCDR3 |
| H4H13290P2 | 2 | 4 | 6 | 8 | 138 | 140 | 142 | 144 |
| H4H13291P2 | 10 | 12 | 14 | 16 | 138 | 140 | 142 | 144 |
| H4H13295P2 | 18 | 20 | 22 | 24 | 138 | 140 | 142 | 144 |
| H4H13299P2 | 26 | 28 | 30 | 32 | 138 | 140 | 142 | 144 |
| H4H13300P2 | 34 | 36 | 38 | 40 | 138 | 140 | 142 | 144 |
| H4H13301P2 | 42 | 44 | 46 | 48 | 138 | 140 | 142 | 144 |
| H4H13302P2 | 50 | 52 | 54 | 56 | 138 | 140 | 142 | 144 |
| H4H13306P2 | 58 | 60 | 62 | 64 | 138 | 140 | 142 | 144 |
| H4H13309P2 | 66 | 68 | 70 | 72 | 138 | 140 | 142 | 144 |
| H4H13311P2 | 74 | 76 | 78 | 80 | 138 | 140 | 142 | 144 |
| H4H13312P2 | 82 | 84 | 86 | 88 | 138 | 140 | 142 | 144 |
| H4H13313P2 | 90 | 92 | 94 | 96 | 138 | 140 | 142 | 144 |
| H4H13316P2 | 98 | 100 | 102 | 104 | 138 | 140 | 142 | 144 |
| H4H13318P2 | 106 | 108 | 110 | 112 | 138 | 140 | 142 | 144 |
| H4H13319P2 | 114 | 116 | 118 | 120 | 138 | 140 | 142 | 144 |
| H4H13325P2 | 122 | 124 | 126 | 128 | 138 | 140 | 142 | 144 |
| H4H13331P2 | 130 | 132 | 134 | 136 | 138 | 140 | 142 | 144 |

TABLE 2
Nucleic Acid Sequence Identifiers

| | SEQ ID NOs: | | | | | | | |
|---|---|---|---|---|---|---|---|---|
| Antibody Designation | HCVR | HCDR1 | HCDR2 | HCDR3 | LCVR | LCDR1 | LCDR2 | LCDR3 |
| H4H13290P2 | 1 | 3 | 5 | 7 | 137 | 139 | 141 | 143 |
| H4H13291P2 | 9 | 11 | 13 | 15 | 137 | 139 | 141 | 143 |
| H4H13295P2 | 17 | 19 | 21 | 23 | 137 | 139 | 141 | 143 |
| H4H13299P2 | 25 | 27 | 29 | 31 | 137 | 139 | 141 | 143 |
| H4H13300P2 | 33 | 35 | 37 | 39 | 137 | 139 | 141 | 143 |
| H4H13301P2 | 41 | 43 | 45 | 47 | 137 | 139 | 141 | 143 |
| H4H13302P2 | 49 | 51 | 53 | 55 | 137 | 139 | 141 | 143 |
| H4H13306P2 | 57 | 59 | 61 | 63 | 137 | 139 | 141 | 143 |
| H4H13309P2 | 65 | 67 | 69 | 71 | 137 | 139 | 141 | 143 |
| H4H13311P2 | 73 | 75 | 77 | 79 | 137 | 139 | 141 | 143 |
| H4H13312P2 | 81 | 83 | 85 | 87 | 137 | 139 | 141 | 143 |
| H4H13313P2 | 89 | 91 | 93 | 95 | 137 | 139 | 141 | 143 |
| H4H13316P2 | 97 | 99 | 101 | 103 | 137 | 139 | 141 | 143 |
| H4H13318P2 | 105 | 107 | 109 | 111 | 137 | 139 | 141 | 143 |
| H4H13319P2 | 113 | 115 | 117 | 119 | 137 | 139 | 141 | 143 |
| H4H13325P2 | 121 | 123 | 125 | 127 | 137 | 139 | 141 | 143 |
| H4H13331P2 | 129 | 131 | 133 | 135 | 137 | 139 | 141 | 143 |

Antibodies are typically referred to herein according to the following nomenclature: Fc prefix (e.g. “H4H”), followed by a numerical identifier (e.g. “13290,” “13291,” “13295,” etc.), followed by a “P2” suffix, as shown in Tables 1 and 2. Thus, according to this nomenclature, an antibody may be referred to herein as, e.g., “H4H13290P2,” “H4H13291P2,” “H4H13295P2,” etc. The prefix on the antibody designations used herein indicate the particular Fc region isotype of the antibody. In particular, an “H4H” antibody has a human IgG4 Fc (all variable regions are fully human as denoted by the first ‘H’ in the antibody designation). As will be appreciated by a person of ordinary skill in the art, an antibody having a particular Fc isotype can be converted to an antibody with a different Fc isotype (e.g., an antibody with a mouse IgG4 Fc can be converted to an antibody with a human IgG1, etc.), but in any event, the variable domains (including the CDRs)—which are indicated by the numerical identifiers shown in Tables 1 and 2—will remain the same, and the binding properties are expected to be identical or substantially similar regardless of the nature of the Fc domain.

### Example 3. Surface Plasmon Resonance Derived Binding Affinities and Kinetic Constants of Human Monoclonal Anti-MET (Monospecific) Antibodies

Binding affinities and kinetic constants of human anti-MET antibodies were determined by surface plasmon resonance (Biacore 4000 or T-200) at 37° C. The anti-Met antibodies tested in this example were bivalent monospecific binders of MET. The antibodies, expressed as human IgG4 (designated “H4H”), were captured onto a CM4 or CM5 Biacore sensor surface derivatized via amine coupling with a monoclonal mouse anti-human Fc antibody (GE, BR-1008-39). Various concentrations of soluble monomeric (human (h) Met.mmh; SEQ ID NO: 152; Macaca fascicularis (mf) Met.mmh; SEQ ID NO: 154) or dimeric (hMet.mFc; SEQ ID NO: 153) Met proteins were injected over the anti-MET-antibody captured surface at a flow rate of 30 or 50 μL/minute. Association of hMET.mmh or hMET.mFc to the captured monoclonal antibody was monitored for 4 or 5 minutes and the dissociation of hMET.mmh or hMET.mFc in HBS-ET (0.01M HEPES pH 7.4, 0.15M NaCl, 3 mM EDTA, 0.05% v/v Surfactant P20) or PBS-P (0.01M Sodium Phosphate pH 7.4, 0.15M NaCl, 0.05% v/v Surfactant P20) running buffer was monitored for 10 minutes.

Kinetic association (kₐ) and dissociation (k_{d}) rate constants were determined by fitting the real-time sensorgrams to a 1:1 binding model using Scrubber 2.0c curve fitting software. Binding dissociation equilibrium constant (K_{D}) and dissociative half-life (t1/2) were calculated from the kinetic rate constants as:

$${K}_{D} ( M ) = \frac{kd}{ka} , \\ and$$

$$t ½ ( min ) = \frac{ln ( 2 )}{60 * kd}$$

Binding kinetic parameters for the monospecific anti-Met antibodies to monomeric and dimeric Met protein are shown below in Table 3.

TABLE 3
Biacore Binding Affinities of Monospecific Anti-MET mAbs at 37° C.
Binding at 37° C./Antibody-Capture Format

| | | ka | kd | K_{D} | T½ |
|---|---|---|---|---|---|
| Antibody | Analyte | (Ms⁻¹) | (s⁻¹) | (Molar) | (min) |
| H4H13290P2 | hMet.mmh | 2.53E+05 | 8.03E−04 | 3.17E−09 | 14.4 |
| | hMET.mFc | 6.15E+05 | 3.15E−04 | 5.13E−10 | 36.6 |
| | mfMet.mmh | 1.23E+05 | 6.33E−04 | 5.16E−09 | 18.2 |
| H4H13291P2 | hMet.mmh | 2.55E+04 | 2.38E−03 | 9.34E−08 | 4.8 |
| | hMET.mFc | 3.33E+05 | 3.39E−04 | 1.02E−09 | 34 |
| | mfMet.mmh | 3.70E+04 | 1.39E−03 | 3.76E−08 | 8.3 |
| H4H13295P2 | hMet.mmh | 1.67E+04 | 5.40E−04 | 3.24E−08 | 21.4 |
| | hMET.mFc | 2.28E+05 | 2.64E−04 | 1.16E−09 | 43.8 |
| | mfMet.mmh | 1.65E+04 | 9.79E−04 | 5.93E−08 | 11.8 |
| H4H13299P2 | hMet.mmh | 9.10E+04 | 7.80E−04 | 8.57E−09 | 14.8 |
| | hMET.mFc | 3.57E+05 | 3.14E−04 | 8.78E−10 | 36.8 |
| | mfMet.mmh | 1.13E+05 | 8.84E−04 | 7.86E−09 | 13.1 |
| H4H13300P2 | hMet.mmh | 3.35E+04 | 2.43E−03 | 7.25E−08 | 4.8 |
| | hMET.mFc | 2.65E+05 | 2.95E−04 | 1.12E−09 | 39.1 |
| | mfMet.mmh | 5.13E+04 | 1.94E−03 | 3.77E−08 | 6.0 |
| H4H13301P2 | hMet.mmh | 7.57E+04 | 6.22E−03 | 8.22E−08 | 1.9 |
| | hMET.mFc | 7.05E+05 | 1.14E−03 | 1.62E−09 | 10.1 |
| | mfMet.mmh | 6.85E+04 | 5.30E−03 | 7.74E−08 | 2.2 |
| H4H13302P2 | hMet.mmh | 5.24E+04 | 2.46E−03 | 4.70E−08 | 4.7 |
| | hMET.mFc | 2.51E+05 | 5.84E−04 | 2.33E−09 | 19.8 |
| | mfMet.mmh | 3.56E+04 | 2.92E−03 | 8.20E−08 | 4.0 |
| H4H13306P2 | hMet.mmh | 1.52E+05 | 1.66E−02 | 1.09E−07 | 0.7 |
| | hMET.mFc | 1.21E+06 | 2.60E−03 | 2.15E−09 | 4.4 |
| | mfMet.mmh | 1.21E+06 | 3.11E−02 | 2.58E−08 | 0.4 |
| H4H13309P2 | hMet.mmh | 9.20E+04 | 5.87E−04 | 6.38E−09 | 19.7 |
| | hMET.mFc | 4.06E+05 | 2.67E−04 | 6.57E−10 | 43.3 |
| | mfMet.mmh | 1.23E+05 | 6.33E−04 | 5.16E−09 | 18.2 |
| H4H13311P2 | hMet.mmh | 4.48E+04 | 5.19E−03 | 1.16E−07 | 2.2 |
| | hMET.mFc | 3.02E+05 | 4.68E−04 | 1.55E−09 | 24.7 |
| | mfMet.mmh | 7.61E+04 | 6.04E−03 | 7.94E−08 | 1.9 |
| H4H13312P2 | hMet.mmh | 7.19E+04 | 1.63E−02 | 2.27E−07 | 0.7 |
| | hMET.mFc | 6.14E+05 | 1.71E−03 | 2.79E−09 | 6.7 |
| | mfMet.mmh | 1.47E+05 | 7.72E−03 | 5.24E−08 | 1.5 |
| H4H13313P2 | hMet.mmh | 8.78E+04 | 5.70E−03 | 6.49E−08 | 2 |
| | hMET.mFc | 7.50E+05 | 8.93E−04 | 1.19E−09 | 12.9 |
| | mfMet.mmh | 5.10E+04 | 4.08E−03 | 8.00E−08 | 2.8 |
| H4H13316P2 | hMet.mmh | 7.82E+04 | 1.51E−03 | 1.93E−08 | 7.6 |
| | hMET.mFc | 2.93E+05 | 1.08E−04 | 3.67E−10 | 107.4 |
| | mfMet.mmh | NB | NB | NB | NB |
| H4H13318P2 | hMet.mmh | 3.30E+04 | 2.92E−03 | 8.83E−08 | 4 |
| | hMET.mFc | 3.52E+05 | 1.65E−04 | 4.67E−10 | 70.2 |
| | mfMet.mmh | NB | NB | NB | NB |
| H4H13319P2 | hMet.mmh | 3.11E+04 | 2.38E−03 | 7.65E−08 | 4.9 |
| | hMET.mFc | 3.82E+05 | 5.42E−04 | 1.42E−09 | 21.3 |
| | mfMet.mmh | 2.66E+04 | 1.15E−03 | 4.33E−08 | 10.0 |
| H4H13325P2 | hMet.mmh | 9.53E+04 | 2.36E−03 | 2.48E−08 | 4.9 |
| | hMET.mFc | 3.06E+05 | 1.85E−04 | 6.05E−10 | 62.4 |
| | mfMet.mmh | NB | NB | NB | NB |
| H4H13331P2 | hMet.mmh | 2.61E+05 | 8.73E−04 | 3.35E−09 | 13.2 |
| | hMET.mFc | 6.39E+05 | 1.56E−04 | 2.44E−10 | 74.1 |
| | mfMet.mmh | 1.61E+05 | 1.04E−03 | 6.47E−09 | 11.1 |

NB = No binding observed under conditions used

As shown in Table 3, several antibodies displayed high affinity binding to human and monkey MET protein.

### Example 4. Anti-Met Antibodies Bind to Distinct Epitopes on Met Receptor

To assess whether two anti-Met antibodies are able to compete with one another for binding to their respective epitopes on MET, a binding competition assay was conducted using real time, label-free bio-layer interferometry (BLI) on an OCTET® HTX biosensor (FortéBio Corp., Menlo Park, Calif.).

Briefly, approximately 0.25 nM of human MET extracellular domain expressed with a C-terminal myc-myc-hexahistidine tag (hMet.mmh) was first captured onto anti-penta-His antibody coated OCTET® biosensors (FortéBio Corp., #18-5079) by submerging the biosensors for 5 minutes into wells containing a 20 μg/mL solution of hMET.mmh. The antigen-captured biosensors were then saturated with the first anti-MET monoclonal antibody (subsequently referred to as mAb-1) by immersion into wells containing a 50 μg/mL solution of mAb-1 for 5 minutes. The biosensors were then submerged into wells containing a 50 μg/mL solution of a second anti-MET monoclonal antibody (subsequently referred to as mAb-2) for 3 minutes. All of the biosensors were washed in OCTET® HEPES-buffered saline-EDTA polysorbate 20 (HBS-EP) buffer in between each step of the experiment. The real-time binding response was monitored during the course of the experiment and the binding response at the end of each step was recorded. The response of mAb-2 binding to anti-MET pre-complexed with mAb-1 was compared and the competitive/non-competitive behavior of the different anti-MET monoclonal antibodies was determined using a 50% inhibition threshold. Table 4 explicitly defines the relationships of antibodies competing in both directions, independent of the order of binding.

TABLE 4
Cross-competition of anti-MET antibodies
for binding to hMET.mmh

| First mAb | | First mAb | |
|---|---|---|---|
| (mAb-1) | mAb-2 | (mAb-1) | mAb-2 |
| Captured using | antibodies | Captured using | antibodies |
| Anti-Penta-His | which Compete | Anti-Penta-His | which Compete |
| Octet Biosensors | with mAb-1 | Octet Biosensors | with mAb-1 |
| H4H13301P2 | H4H13302P2 | H4H13300P2 | H4H13291P2 |
| H4H13302P2 | H4H13301P2 | | H4H13295P2 |
| H4H13290P2 | H4H13306P2 | | H4H13311P2 |
| | H4H13316P2 | | H4H13318P2 |
| H4H13306P2 | H4H13290P2 | | H4H13319P2 |
| | H4H13316P2 | H4H13311P2 | H4H13291P2 |
| H4H13316P2 | H4H13290P2 | | H4H13295P2 |
| | H4H13306P2 | | H4H13300P2 |
| | H4H13325P2 | | H4H13318P2 |
| | H4H13331P2 | | H4H13319P2 |
| H4H13325P2 | H4H13316P2 | H4H13318P2 | H4H13291P2 |
| | H4H13331P2 | | H4H13295P2 |
| H4H13312P2 | H4H13331P2 | | H4H13300P2 |
| H4H13291P2 | H4H13295P2 | | H4H13311P2 |
| | H4H13300P2 | | H4H13319P2 |
| | H4H13311P2 | H4H13319P2 | H4H13291P2 |
| | H4H13318P2 | | H4H13295P2 |
| | H4H13319P2 | | H4H13300P2 |
| H4H13295P2 | H4H13291P2 | | H4H13311P2 |
| | H4H13300P2 | | H4H13318P2 |
| | H4H13311P2 | H4H13331P2 | H4H13316P2 |
| | H4H13318P2 | | H4H13325P2 |
| | H4H13319P2 | | H4H13312P2 |

### Example 5. Construction of Bispecific Antibodies Having Two Different Antigen-Binding Domains Specific for Different Epitopes of MET

This example describes the construction of bispecific antibodies comprising two different antigen-binding domains (D1 and D2), wherein D1 and D2 are derived from different anti-MET antibodies and, consequently, bind to separate epitopes on the MET extracellular domain.

The individual anti-MET antigen-binding domains used to construct the bispecific antibodies of this Example were derived from various bivalent, monospecific anti-MET antibodies described in Examples 1 through 3, herein. All anti-MET antibodies described herein comprise the same (“common”) light chain (comprising the light chain variable region [LCVR] amino acid sequence of SEQ ID NO:138, and light chain CDR [LCDR1, LCDR2 and LCDR3] amino acid sequences of SEQ ID NOs: 140, 142 and 144). In addition, all of the bispecific antibodies illustrated in this Example contain a “D2” arm derived from the exemplary anti-MET antibody H4H13312P2. Thus, both antigen-binding domains (D1 and D2) of all of the bispecific antibodies described in this example comprise this common light chain variable region, and all D2 binding arms comprise the heavy chain variable region from H4H13312P2; however, the bispecific antibodies differ from one another in terms of their D1 heavy chain variable regions (HCVRs) and heavy chain CDRs (HCDRs). The components of the bispecific antibodies of this Example are summarized in Table 5.

TABLE 5
MET x MET Bispecific Antibody Components Summary

| | SEQ ID NOs: (Amino Acid Sequences) | | | | | | | |
|---|---|---|---|---|---|---|---|---|
| | First Antigen-Binding Domain (D1) | | | | Second Antigen-Binding Domain (D2) | | | |
| Bispecific | D1- | D1- | D1- | D1- | D2- | D2- | D2- | D2- |
| Antibody | HCVR | HCDR1 | HCDR2 | HCDR3 | HCVR | HCDR1 | HCDR2 | HCDR3 |
| H4H14634D | H4H13290P2 | | | | H4H13312P2 | | | |
| (No. 10) | 2 | 4 | 6 | 8 | 82 | 84 | 86 | 88 |
| H4H14635D | H4H13295P2 | | | | H4H13312P2 | | | |
| (No. 42) | 18 | 20 | 22 | 24 | 82 | 84 | 86 | 88 |
| H4H14636D | H4H13299P2 | | | | H4H13312P2 | | | |
| (No. 74) | 26 | 28 | 30 | 32 | 82 | 84 | 86 | 88 |
| H4H14637D | H4H13301P2 | | | | H4H13312P2 | | | |
| (No. 90) | 42 | 44 | 46 | 48 | 82 | 84 | 86 | 88 |
| H4H14638D | H4H13302P2 | | | | H4H13312P2 | | | |
| (No. 106) | 50 | 52 | 54 | 56 | 82 | 84 | 86 | 88 |
| H4H14639D | H4H13306P2 | | | | H4H13312P2 | | | |
| (No. 122) | 58 | 60 | 62 | 64 | 82 | 84 | 86 | 88 |
| H4H14640D | H4H13309P2 | | | | H4H13312P2 | | | |
| (No. 138) | 66 | 68 | 70 | 72 | 82 | 84 | 86 | 88 |
| H4H14641D | H4H13313P2 | | | | H4H13312P2 | | | |
| (No. 187) | 90 | 92 | 94 | 96 | 82 | 84 | 86 | 88 |
| H4H16445D | H4H13291P2 | | | | H4H13312P2 | | | |
| (No. 26) | 10 | 12 | 14 | 16 | 82 | 84 | 86 | 88 |
| H4H16446D | H4H13300P2 | | | | H4H13312P2 | | | |
| (No. 58) | 34 | 36 | 38 | 40 | 82 | 84 | 86 | 88 |
| H4H16447D | H4H13311P2 | | | | H4H13312P2 | | | |
| (No. 154) | 74 | 76 | 78 | 80 | 82 | 84 | 86 | 88 |
| H4H16448D | H4H13318P2 | | | | H4H13312P2 | | | |
| (No. 219) | 106 | 108 | 110 | 112 | 82 | 84 | 86 | 88 |
| H4H16449D | H4H13319P2 | | | | H4H13312P2 | | | |
| (No. 235) | 114 | 116 | 118 | 120 | 82 | 84 | 86 | 88 |

* The number designation in parentheses under the bispecific antibody identifiers (e.g., “No. 10”) indicates the bispecific antibody number depicted in the MET x MET bispecific antibody matrix of FIG. 1.

### Example 6. Surface Plasmon Resonance Derived Binding Affinities and Kinetic Constants of MET×MET Human Bispecific Monoclonal Antibodies

Binding affinities and kinetic constants of the MET×MET bispecific antibodies constructed in accordance with Example 4 herein were determined by surface plasmon resonance (Biacore 4000 or T-200) at 37° C. The bispecific antibodies, expressed as human IgG4 (designated “H4H”), were captured onto a CM4 or CM5 Biacore sensor surface derivatized via amine coupling with a monoclonal mouse anti-human Fc antibody (GE, BR-1008-39). Various concentrations of soluble monomeric MET protein (hMet.mmh, SEQ ID NO: 152) were injected over the anti-MET×MET bispecific antibody-captured surface at a flow rate of 30 or 50 μL/minute. Association of the analyte to the captured bispecific antibody was monitored for 4 or 5 minutes and the dissociation of the analyte in HBS-ET (0.01M HEPES pH 7.4, 0.15M NaCl, 3 mM EDTA, 0.05% v/v Surfactant P20) or PBS-P (0.01M Sodium Phosphate pH 7.4, 0.15M NaCl, 0.05% v/v Surfactant P20) running buffer was monitored for 10 minutes.

Kinetic association (kₐ) and dissociation (k_{d}) rate constants were determined as described in Example 3.

Binding kinetic parameters for the bispecific anti-Met antibodies to monomeric Met protein (hMET.mmh) are shown in Table 6.

TABLE 6
Biacore Binding Affinities of Bispecific Anti-MET mAbs at 37° C.
Binding at 37° C./Antibody-Capture Format

| Bispecific | | ka | kd | K_{D} | T½ |
|---|---|---|---|---|---|
| Antibody | Analyte | (Ms⁻¹) | (s⁻¹) | (Molar) | (min) |
| H4H14634D | hMet.mmh | N/A | ≤1E−5 | N/A | ≥1155 |
| H4H14635D | hMet.mmh | N/A | 8.21E−05 | N/A | 140.6 |
| H4H14636D | hMet.mmh | N/A | ≤1E−5 | N/A | ≥1155 |
| H4H14637D | hMet.mmh | N/A | 3.26E−04 | N/A | 35.4 |
| H4H14638D | hMet.mmh | N/A | 1.65E−04 | N/A | 70.2 |
| H4H14639D | hMet.mmh | N/A | 1.63E−04 | N/A | 70.8 |
| H4H14640D | hMet.mmh | N/A | ≤1E−5 | N/A | ≥1155 |
| H4H14641D | hMet.mmh | N/A | 3.27E−04 | N/A | 35.3 |
| H4H16445D | hMet.mmh | N/A | 3.93E−04 | N/A | 29.4 |
| H4H16446D | hMet.mmh | N/A | 1.03E−04 | N/A | 111.8 |
| H4H16447D | hMet.mmh | N/A | 8.48E−04 | N/A | 13.6 |
| H4H16448D | hMet.mmh | N/A | 5.92E−04 | N/A | 19.5 |
| H4H16449D | hMet.mmh | N/A | 2.94E−04 | N/A | 39.3 |

As shown in Table 6, the bispecific “MET×MET” antibodies described herein exhibited T 1/2 values of up to greater than 1155 minutes.

As shown in Table 7, the dissociation rate for the bispecific antibody H4H14639D is significantly lower than the dissociation rates of each of its parental antibodies, H4H13306P2 and H4H13312P2.

TABLE 7
Biacore Binding Affinities of Bispecific
Anti-MET mAb and Monospecific Parents at 37° C.
Binding at 37° C./Antibody-Capture Format

| Antibody | Analyte | kd (s⁻¹) | T½ (min) |
|---|---|---|---|
| H4H13306P2 | hMet.mmh | 1.66E−02 | 0.7 |
| H4H13312P2 | hMet.mmh | 8.40E−03 | 1.4 |
| H4H14639D | hMet.mmh | 1.63E−04 | 70.8 |

### Example 7. Anti-Met Antibodies Block HFG-Mediated Met Activation in SRE-Luciferase Reporter Bioassay

The ability of anti-MET antibodies to block hepatocyte growth factor (HGF)-mediated MET activation was examined in a luciferase-based reporter assay. The growth factor HGF binds to the extracellular domain of its receptor c-Met (MET), triggering rapid homodimerization and activating several downstream signaling cascades. The anti-MET antibodies tested in this example were bivalent monospecific binders of MET, or anti-MET “bispecifics”, in which each arm of the bispecific antibody bound to a different and distinct epitope on MET.

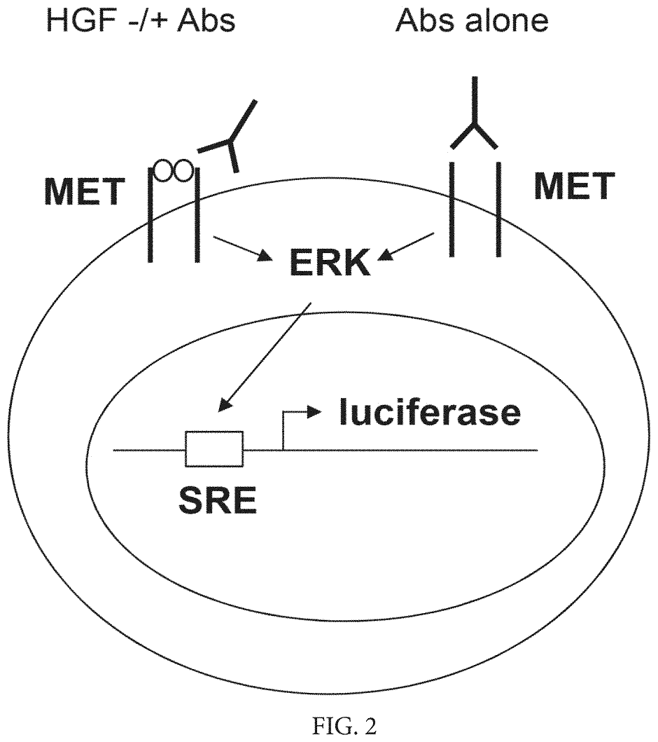

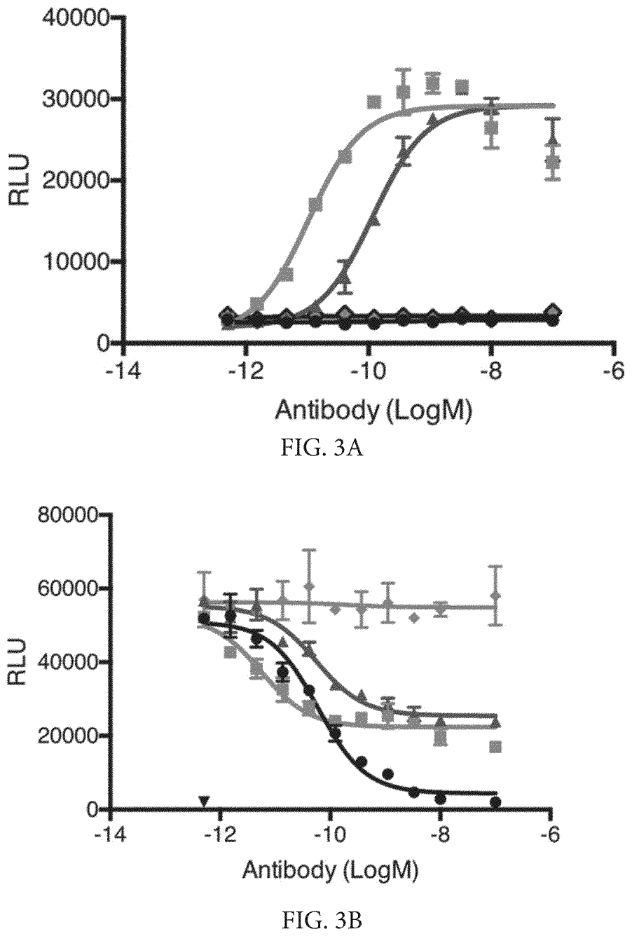

An engineered cell-based luciferase reporter assay (FIG. 2) was used to determine the ability of anti-MET antibodies to activate MET signaling (FIG. 3, panel A; Table 8, columns 3 and 4) and to block ligand-mediated activation of MET (FIG. 3, panel B; Table 8, columns 1 and 2). Briefly, the CIGNAL™ Lenti SRE Reporter (luc) Kit (SABiosciences, Hilden, Del.) was used to generate HEK293/SRE-Luc cells. HEK293 (human embryonic kidney) cells were selected because they endogenously express c-Met. The HEK293/SRE-Luc cells stably incorporated the serum response element (SRE)-dependent luciferase (Luc) reporter (see Dinter et al., PLoS ONE 10(2): e0117774, 2015). HEK293/SRE-Luc cells were cultured in DMEM supplemented with 10% fetal bovine serum (FBS), penicillin/streptomycin/glutamine, and 1 μg/ml puromycin.

Next, 2.0×10⁵ HEK293/SRE-Luc cells were seeded in luciferase assay media in 96 well plates and incubated overnight at 37° C. in 5% CO₂. Hepatocyte growth factor (HGF) dose response curves were generated by adding serially diluted HGF (0.01 μM to 1.0 nM) to cells and recording the luciferase signal after incubation at 37° C. for four to six hours in the absence of antibodies. To generate antibody inhibition curves, cells were pre-incubated for one hour at 37° C. with serially diluted anti-human MET antibodies (1.1 μM to 200 nM). HGF at a concentration of 73 μM or 100 μM was then added for an additional four to six hours before recording the signal. Separately, the ability of the antibodies to activate c-Met in the absence of ligand was also assessed.

Luciferase activity was detected using the ONE-Glo™ Luciferase Assay System (Promega, Madison, Wis.), and emitted light was measured on a Victor or Envision luminometer (Perkin Elmer, Shelton, Conn.) and expressed as relative light units (RLUs). EC50/1050 values were determined from a four-parameter logistic equation over a 12-point response curve using GRAPHPAD PRISM®. Percent HGF blocking and fold MET activation (mAbs alone) were reported for the highest antibody dose. The results are shown in Table 8.

TABLE 8
Anti-Met Antibody Blocking of HGF-Mediated Signaling
and Activation of SRE-Luc in the Absence of Ligand

| | HEK293/SRE-Luc | | Ligand (HGF)- | |
|---|---|---|---|---|
| | Blocking Activity | | Independent HEK293/ | |
| | (1 h mAb pre-bind) | | SRE-Luc Activation | |
| | % Inhi- | IC₅₀ | Fold | EC₅₀ |
| Antibody ID | bition | (M) | Response | (M) |

Anti-MET Bivalent Monospecific antibodies
Antibodies expressed with a hIgG1-Fc

| | | | | |
|---|---|---|---|---|
| H1H13301P2 | 42 | 3.3E−09 | 1.4 | ND |
| H1H13316P2 | 86 | 4.0E−11 | 1.7 | ND |

Antibodies expressed with a hIgG4-Fc

| | | | | |
|---|---|---|---|---|
| H4H13312P2 | 48 | 7.7E−11 | 10.9 | 1.2E−10 |
| H4H13325P2 | 69 | 1.3E−11 | 4.3 | 1.9E−10 |
| H4H13316P2 | 74 | 7.8E−12 | 2.3 | 4.7E−11 |
| H4H13302P2 | 45 | 1.6E−09 | 1.8 | ND |
| H4H13313P2 | 47 | 2.3E−09 | 1.2 | ND |
| H4H13301P2 | 40 | 1.5E−09 | 1.6 | ND |
| H4H13295P2 | 70 | 5.5E−11 | 2.8 | 3.0E−10 |
| H4H13306P2 | 67 | ND | 9.8 | 1.3E−11 |
| H4H13291P2 | 61 | 1.3E−10 | 2.7 | 3.9E−10 |
| H4H13319P2 | 67 | 5.2E−11 | 4.8 | 1.8E−10 |
| H4H13309P2 | 77 | 2.0E−10 | 9.2 | 3.9E−10 |
| H4H13318P2 | 77 | 1.0E−10 | 3.1 | ND |
| H4H13300P2 | 69 | 1.2E−10 | 2.8 | 4.8E−10 |
| H4H13290P2 | 56 | <2.0E−12 | 9.8 | <2.0E−12 |
| H4H13311P2 | 62 | 3.5E−11 | 5.2 | 3.0E−10 |
| H4H13331P2 | 75 | <1.0E−11 | 7.1 | 2.3E−12 |
| H4H13299P2 | 51 | ND | 14.4 | 3.7E−12 |

Anti-MET Bispecific Antibodies (hIgG4-Fc)

| | | | | |
|---|---|---|---|---|
| H4H14639D | 95 | 2.4E−11 | 1.8 | 5.7E−11 |
| H4H14640D | 89 | 5.2E−10 | 2.5 | 6.8E−09 |
| H4H14634D | 85 | 9.7E−12 | 3.4 | 9.0E−11 |
| H4H14635D | 85 | 1.9E−10 | 2.2 | 1.4E−09 |
| H4H14638D | 79 | 1.1E−09 | 2.6 | 5.9E−09 |
| H4H14641D | 75 | 2.7E−09 | 4.4 | 8.4E−08 |
| H4H14636D | 74 | ND | 2.8 | 2.8E−10 |
| H4H14637D | 73 | ND | 2.1 | 4.1E−09 |
| H4H16445D | 81 | 5.2E−10 | 4.3 | 1.0E−09 |
| H4H16446D | 83 | 1.0E−09 | 4.0 | 1.4E−09 |
| H4H16447D | 76 | 8.6E−10 | 5.8 | 1.4E−09 |
| H4H16448D | 87 | 6.2E−10 | 4.3 | 9.1E−10 |
| H4H16449D | 85 | 3.2E−10 | 4.2 | 4.2E−10 |

NT = not tested; ND = EC50/IC50 not determined due to non-sigmoidal curves or incomplete blocking.

As summarized in Table 8, a majority of the antibodies inhibited activation of the SRE reporter, with IC50 values ranging from <2.0 μM to about 1.0 nM. Several exemplary monospecific bivalent anti-MET antibodies, such as H4H13306P2 and H4H13309P2, were potent inhibitors of SRE-luc activation, with percent inhibition values of 67% and 77%, respectively. Anti-MET bispecific antibodies (MET×MET) exhibited greater inhibition of SRE-luc activation overall. For example, MET×MET bispecific antibody H4H14639D displayed 95 percent inhibition. Additionally, several blocking antibodies were weakly activating in the absence of ligand with fold activation responses ranging from 0.8 to 14.4 above baseline levels.

Also as shown in FIG. 3, the bivalent monospecific antibodies H41413306P2 and H4H13312P2 each activate the Met pathway in the absence of HGF ligand (panel A) and also block HGF activation of the Met (panel B).

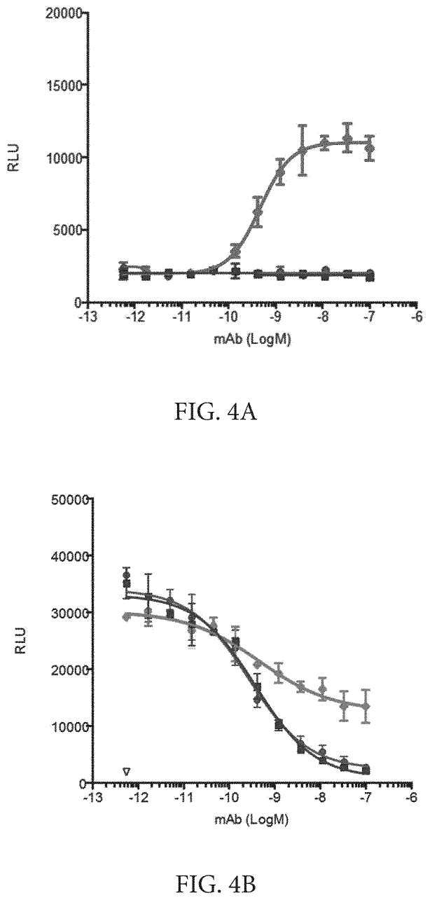

The effect of a bispecific MET×MET antibody (e.g., H4H14639D) on HGF-dependent and HGF-independent MET activation was also assessed using the HEK293/SRE-Luc system. SRE-driven Luciferase activity was measured in HEK293T cells treated with the MET antibodies H4H14639D (the MET×MET bispecific antibody), a monovalent anti-MET antibody, and the H4H14639D parental antibody H4H13312P2 at various concentrations to ascertain the level of HGF-independent MET agonism. While the parental anti-MET monospecific bivalent antibody showed MET agonist activity, neither the monovalent nor the MET×MET bispecific antibody showed MET agonist activity (FIG. 4, panel A).

SRE-driven Luciferase activity was measured in HEK293T cells treated with the MET antibodies H4H14639D (the MET×MET bispecific antibody), a monovalent anti-MET antibody, and the H4H14639D parental antibody H4H13312P2 at various concentrations to ascertain the level of inhibition or blocking of HGF-dependent MET agonism. While the parental anti-MET monospecific bivalent antibody showed some HGF blocking activity, both the monovalent and the MET×MET bispecific antibody showed greater HGF blocking (FIG. 4, panel B).

The MET×MET bispecific antibody blocks HGF signaling and exhibits low MET agonist activity.

### Example 8. Anti-Met Antibodies Inhibit Growth of Met-Amplified Cells

Next, selected anti-Met antibodies were tested for their ability to inhibit the growth of MET-amplified SNU5 cells. Briefly, 2.5×10³ human gastric carcinoma (SNU5) cells were seeded in complete growth media in the presence of anti-MET antibodies at concentrations ranging from 1.5 μM to 100 nM. The SNU5 complete growth media contained Iscove's Modified Dulbecco's Medium, 10% FBS, and penicillin/streptomycin/glutamine. Cells were incubated for 5 days and the number of viable cells was determined using the CELLTITER-GLO® Luminescent Cell Viability Assay kit (Promega, Madison, Wis.) according to manufacturer instructions.

As summarized in Table 9, several anti-MET antibodies, such as H4H13312P2 and H4H13325P2 blocked SNU5 growth by more than 50%, with overall IC50s ranging from 44 μM to 780 μM.

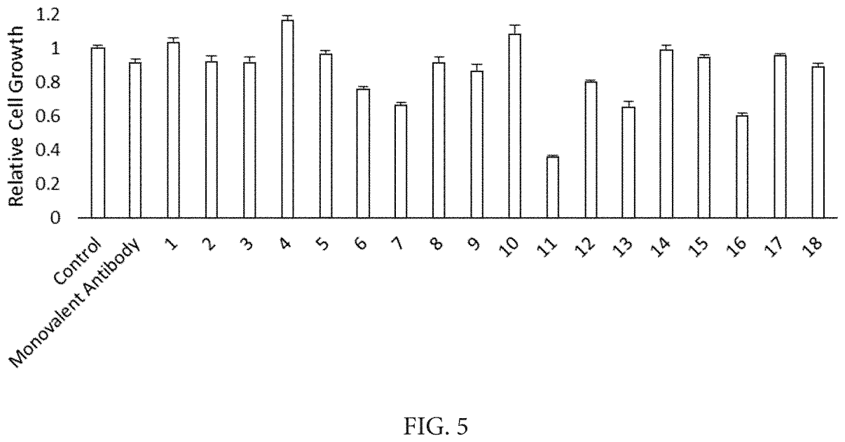

FIG. 5 depicts the relative cell growth of SNU5 cells treated with various anti-MET bivalent monospecific antibodies (i.e., conventional antibodies). A subset of conventional MET antibodies inhibit the growth of SNU5 MET-amplified gastric cancer cells (FIG. 5). SNU5 cells in 96 well plates were treated with each antibody at 10 μg/ml and cell growth was determined after 5 days by reduction of ALAMARBLUE® reagent (Thermo Fisher Scientific, Waltham, Mass.). The monovalent MET antibody (column 2, FIG. 5) was generated using the heavy and light chain variable sequences of MetMab as set forth in U.S. Pat. No. 7,892,550 B2, which is herein incorporated by reference in its entirety. Conventional antibody 8 is H4H13306P2, and conventional antibody 11 is H4H13312P2, which were used to construct the MET×MET bispecific antibody H4H14639D.

In a separate growth assay, the blocking activity of a MET×MET bispecific antibody (i.e., H4H14639D) was assessed in both SNU5 and the non-small cell lung cancer (NSCLC) cell line EBC-1, which also exhibits amplified Met gene and overexpresses MET (Lutterbach et al., Cancer Res. 67(5): 2081-2088, 2007). Complete growth media for the EBC-1 cells contained MEM Earle's Salts, 10% fetal bovine serum (FBS), penicillin/streptomycin/glutamine, and non-essential amino acids for MEM. H4H14369D exhibited the greatest percent inhibition in MET activity according to the SRE-Luciferase read-out. In the current experiment, 3.0×10³ SNU5 or EBC-1 cells were seeded in complete growth media in the presence of H4H14639D at concentrations ranging from 15 μM to 100 nM. Cells were incubated for 3 days at 37° C. in 5% CO₂. The cells were then fixed in 4% formaldehyde and stained with 3 μg/ml Hoechst 33342 to label the nuclei. Images were acquired on the IMAGEXPRESS® Micro XL (Molecular Devices, Sunnyvale, Calif.) and nuclear counts were determined via METAXPRESS® Image Analysis software (Molecular Devices, Sunnyvale, Calif.). Background nuclear counts from cells treated with 40 nM digitonin were subtracted from all wells and viability was expressed as a percentage of the untreated controls. IC50 values were determined from a four-parameter logistic equation over a 10-point response curve (GRAPHPAD PRISM®). IC50 values and percent cell killing are shown in Table 9.

TABLE 9
Anti-MET Antibody Blocking of SNU5 Growth

| Antibody | % Growth Inhibition | IC₅₀ (M) |
|---|---|---|
| H4H13312P2 | 69 | 7.8E−10 |
| H4H13325P2 | 57 | 4.4E−11 |
| H4H13316P2 | 53 | 1.0E−10 |
| H4H13302P2 | 40 | 1.1E−10 |
| H4H13313P2 | 34 | 4.4E−11 |
| H4H13301P2 | 33 | 7.4E−11 |
| H1H13301P2 | 33 | 1.0E−10 |
| H1H13316P2 | 30 | 2.0E−10 |
| H4H13295P2 | 30 | ND |
| H4H13306P2 | 28 | 7.1E−11 |
| H4H13291P2 | 24 | ND |
| H4H13319P2 | 23 | 1.0E−10 |
| H4H13309P2 | 22 | 1.0E−10 |
| H4H13318P2 | 18 | 5.1E−11 |
| H4H13300P2 | 16 | ND |
| H4H13290P2 | 12 | ND |
| H4H13311P2 | 8 | ND |
| H4H13331P2 | 5 | ND |
| H4H13299P2 | −8 | ND |

ND = IC₅₀ not determined due to non-sigmoidal curves or incomplete blocking

As summarized in Table 10, below, the MET×MET bispecific antibody H4H14639D inhibited growth of EBC-1 and SNU5 cells by 37 and 40 percent, and with IC50s of 0.82 nM and 0.3 nM, respectively.

TABLE 10
Anti-Met Bispecific Antibody Blocks EBC-1 and SNU5 Growth

| | |
|---|---|
| IC₅₀ (nM) | % Growth Inhibition |

| mAb | EBC-1 | SNU5 | EBC-1 | SNU5 |
|---|---|---|---|---|
| H4H14639D | 0.82 | 0.30 | 37 | 40 |

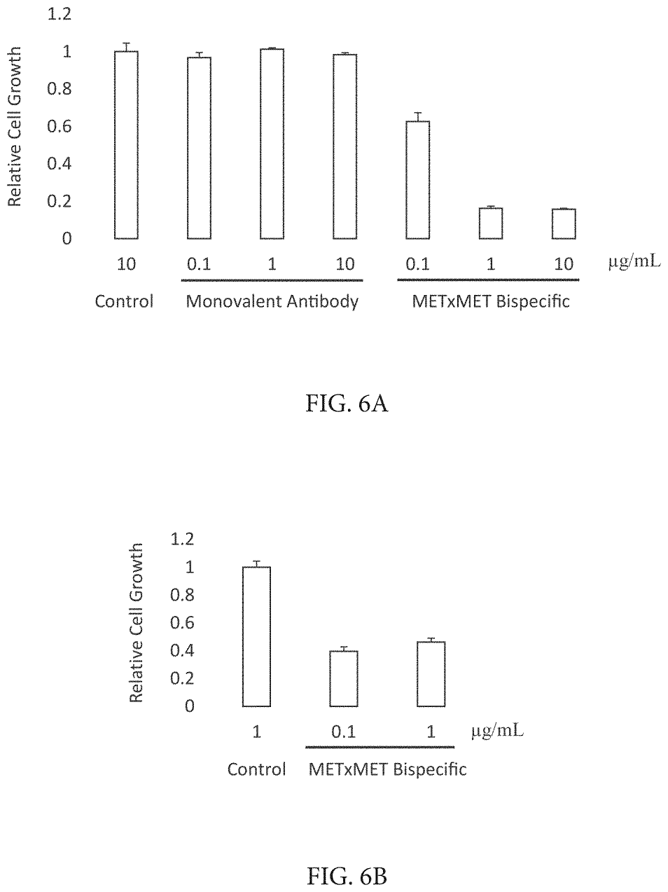

SNU5 cells (gastric) in 96 well plates were treated with a control antibody, a monovalent MET antibody or a MET×MET bispecific antibody at 0.1 μg/mL, 1 μg/mL, or 10 μg/mL. Cell growth was determined after 5 days by reduction of ALAMARBLUE® reagent (Thermo Fisher Scientific, Waltham, Mass.). The MET×MET bispecific antibody significantly reduced the relative cell growth of SNU5 cells compared to the control and monovalent antibody (FIG. 6, panel A).

Likewise, the effect of MET×MET bispecific antibody on the growth of EBC-1 cells was assessed. 2,500 EBC-1 cells were seeded in a 96 well plate and cultured in Dulbecco's Media supplemented with 10% FBS. The cells were treated with a control antibody or a MET×MET bispecific antibody at 0.1 μg/mL or 1 μg/mL, and were subsequently incubated with 5% CO₂ at 37° C. After 5 days, relative cell growth was determined by measuring the reduction of the indicator dye ALAMARBLUE® to its highly fluorescent form in a SPECTRAMAX® M3 plate reader (Molecular Devices, LLC, Sunnyvale, Calif.). The results are shown in Table 11 and FIG. 6, panel B. The MET×MET bispecific antibody (H4H14639D) significantly reduced the relative cell growth of EBC-1 cells compared to the control antibody (FIG. 6, panel B).

Several anti-MET antibodies, both bivalent monospecific and MET×MET bivalent, are potent inhibitors of SRE-Luc activation and inhibit the growth of Met-amplified and MET-overexpressing cell lines.

TABLE 11
Anti-Met Bispecific Antibody Blocks EBC-1 Cell Growth

| | Relative Cell Growth | Standard |
|---|---|---|
| | (n = 3) | Deviation |
| Control | 1.000 | 0.045 |
| 0.1 μg/mL H4H14639D | 0.397 | 0.032 |
| 1 μg/mL H4H14639D | 0.462 | 0.028 |

### Example 9. A MET×MET Bispecific Antibody Induces Modest and Transient MET Pathway Activity in NCI-H596 NSCLC Cells

The effect of a MET×MET bispecific antibody on the MET pathway in human lung adenosquamous carcinoma cells was assessed in vitro.

250,000 NCI-H596 cells were seeded in a 12 well plate and cultured in RPMI Media supplemented with 10% FBS. The cells were treated with hepatocyte growth factor (HGF) at 50 ng/ml or the MET×MET bispecific antibody H4H14639D at 10 μg/ml in duplicate. The cells were subsequently incubated in 5% CO₂ at 37° C. After 0, 2, 6 or 18 hours, cell lysates were prepared, protein content was normalized and immunoblot analysis was performed. MET phosphorylation and ERK phosphorylation were quantified with the ImageJ image processing program (T. Collins, BioTechniques 43: S25-S30, 2007). Phosphorylation levels were normalized to the Tubulin loading control and are expressed as fold change relative to control treatment. The results are summarized in Table 12.

TABLE 12
Phosphorylation of MET and ERK

| | Phospho-MET | Phospho-ERK |
|---|---|---|
| Treatment (hours) | (mean ± SD) | (mean ± SD) |
| Control (hFc) (18) | 1.0 ± 0.5 | 1.0 ± 0.3 |
| HGF (2) | 202.3 ± 38.7 | 16.7 ± 1.6 |
| HGF (6) | 38.9 ± 4.9 | 12.4 ± 3.9 |
| HGF (18) | 59.2 ± 24.4 | 12.4 ± 0.9 |
| H4H14639D (2) | 69.7 ± 7.0 | 2.2 ± 0.9 |
| H4H14639D (6) | 9.9 ± 7.4 | 0.3 ± 0.4 |
| H4H14639D (18) | 1.4 ± 0.1 | 0.1 ± 0.1 |

HGF treatment of NCI-H596 cells induced strong activation of MET and ERK that peaked at 2 hours and was sustained after 18 hours. Modest MET and ERK phosphorylation was detected with the H4H14636D bispecific antibody treatment, which returned to baseline levels by 18 or 6 hours, respectively.

### Example 10. A MET×MET Bispecific Antibody Induces MET Degradation and Inhibits Pathway Activity More Potently than Monospecific Antibodies in Hs746T Gastric Cancer Cells

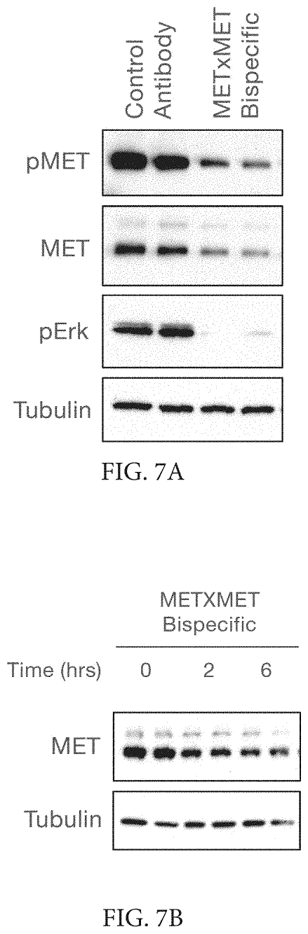

The effect of a MET×MET bispecific antibody on MET activity of human gastric carcinoma cells was assessed in vitro. 250,000 Hs746T human gastric carcinoma cells (H. Smith, J. Nat'l. Cancer Inst. 62(2): 225-230, 1979) were seeded in a 12-well plate and cultured in Modified Dulbecco's Media supplemented with 10% FBS. The cells were treated with (1) 5 μg/ml of the hFc control molecule, (2) 5 μg/ml of the parental bivalent monospecific anti-MET antibody H4H13306P2, (3) 5 μg/ml of the parental bivalent monospecific anti-MET antibody H4H13312P2, (4) the combination of 2.5 μg/mL of H4H13306P2 and 2.5 μg/mL of H4H13312P2, or (5) 5 μg/ml of the MET×MET bispecific antibody H4H14639D. The cells were subsequently incubated with 5% CO₂ at 37° C. After 18 hours, cell lysates were prepared, protein content was normalized and immunoblot analysis was performed. MET expression, MET phosphorylation, and ERK phosphorylation were quantified with the ImageJ image processing program (T. Collins, BioTechniques 43: S25-S30, 2007). The results are summarized in Table 13 and FIG. 7, panel A, which depicts the raw immunoblot data. Panel B of FIG. 7 depicts MET protein expression in cells that were treated with MET×MET bispecific antibody at 10 μg/ml for 0, 2 or 6 hrs. The total MET levels in Hs747T cells declined over time upon treatment with the MET×MET bispecific antibody. Similar results were obtained for the MET amplified human papillary adenocarcinoma NCI-H820 cell line (Bean et al., “MET amplification occurs with or without T790M mutations in EGFR mutant lung tumors with acquired resistance to getfitnib or erlotinib,” Proc. Natl. Acad. Sci. 2007 Dec. 26, 104(52): 20932-20937).

TABLE 13
Relative Levels of MET Protein
and MET/ERK Pathway Activation

| | Relative level | Relative level | Relative level |
|---|---|---|---|
| | MET protein | Phospho-MET | Phospho-ERK |
| Molecule | (mean ± SD) | (mean ± SD) | (mean ± SD) |
| Control (hFc) | 1.00 ± 0.06 | 1.00 ± 0.06 | 1.00 ± 0.03 |
| H4H13306P2 | 0.61 ± 0.09 | 0.57 ± 0.02 | 0.41 ± 0.03 |
| H4H13312P2 | 1.15 ± 0.19 | 0.93 ± 0.04 | 0.39 ± 0.11 |
| H4H13306P2 + | 1.06 ± 0.02 | 1.07 ± 0.10 | 1.04 ± 0.23 |
| H4H13312P2 | | | |
| H4H14639D | 0.41 ± 0.02 | 0.20 ± 0.01 | 0.04 ± 0.01 |

The bispecific antibody, H4H14639D, induced MET degradation more potently than its parental conventional antibodies. Both MET and ERK phosphorylation were more effectively inhibited by treatment with H4H14636D than with the parental antibodies or the combination of the parental antibodies.

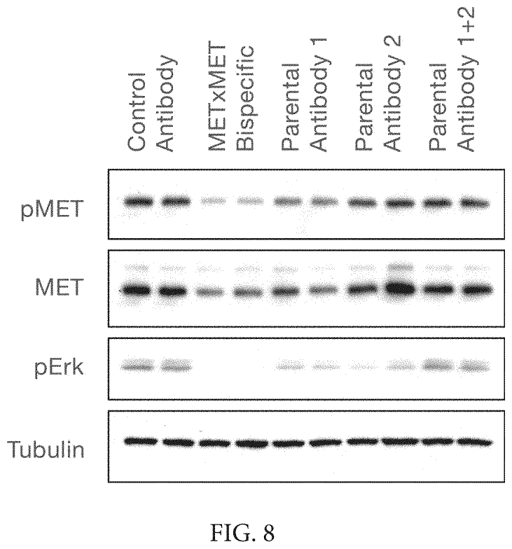

Hs746T gastric cancer cells were treated with control antibody, the MET×MET bispecific antibody H4H14639D, the anti-MET parental antibody H4H13306P2, the anti-MET parental antibody H4H13312P2, and the combination of parental antibodies 1 and 2, each antibody at 10 μg/ml or the combination of parental antibodies at 5 μg/ml each, for 18 hrs. MET expression (MET) and pathway activation (pMET and pErk) were determined by immunoblotting with the indicated antibodies (FIG. 8). MET×MET bispecific antibody inhibits MET pathway activation more effectively than its parental antibodies in Hs746T gastric cancer cells.

### Example 11. A MET×MET Bispecific Antibody Induces MET Degradation More Potently than Monospecific Antibodies in NCI-H596 Lung Cancer Cells

The effect of a MET×MET bispecific antibody and the parental bivalent monospecific anti-MET antibodies on the expression levels of hepatocyte growth factor receptor (HGFR or MET) on human lung adenosquamous carcinoma cells was assessed. 250,000 NCI-H596 human lung adenosquamous carcinoma cells were seeded in a 12-well plate and cultured in RPMI Media supplemented with 10% FBS. The cells were treated with (1) 5 μg/ml of the hFc control molecule, (2) 5 μg/ml of the parental bivalent monospecific anti-MET antibody H4H13306P2, (3) 5 μg/ml of the parental bivalent monospecific anti-MET antibody H4H13312P2, (4) the combination of 2.5 μg/mL of H4H13306P2 and 2.5 μg/mL of H4H13312P2, or (5) 5 μg/ml of the MET×MET bispecific antibody H4H14639D. The cells were subsequently incubated with 5% CO₂ at 37° C. After 18 hours, cell lysates were prepared, protein content was normalized and immunoblot analysis was performed. MET expression was quantified with the ImageJ image processing program (T. Collins, BioTechniques 43: S25-S30, 2007). The results are summarized in Table 14.

TABLE 14
Relative Level of MET Protein

| Molecule | Relative MET Level |
|---|---|
| Control (hFc) | 1 ± 0.03 |
| H4H13306P2 | 0.50 ± 0.01 |
| H4H13312P2 | 0.35 ± 0.04 |
| H4H13306P2 + | 0.61 ± 0.04 |
| H4H13312P2 | |
| H4H14639D | 0.24 ± 0.01 |

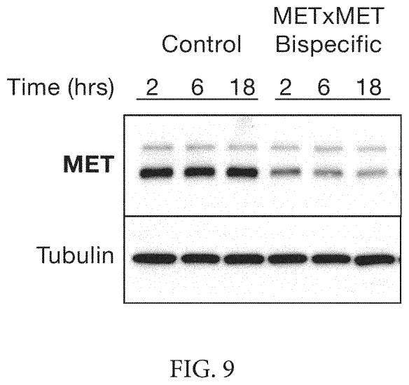

NCI-H596 (MET exon14 skip mutation) lung cancer cells were also treated with control or MET×MET bispecific antibodies at 10 μg/ml for 2, 6 or 18 hrs. MET expression was determined by immunoblotting (FIG. 9), which shows the MET×MET bispecific antibody-induced degradation of MET with increasing time of treatment.

The bispecific antibody, H4H14636D, induces MET degradation more potently than its parental conventional antibodies in NCI-H596 lung cancer cells.

### Example 12. MET×MET Bispecific Antibodies Induce MET Degradation and Inhibit Pathway Activity More Potently than Monospecific Antibodies in SNU5 Gastric Cancer Cells

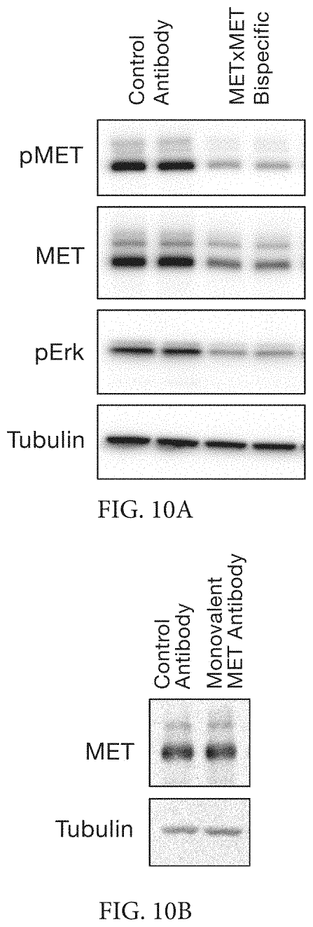

The effect of a bivalent monospecific anti-MET antibody and several MET×MET bispecific antibodies on the expression levels of hepatocyte growth factor receptor (HGFR or MET) on gastric carcinoma cells was assessed. Human gastric carcinoma SNU5 cells were plated in Iscove's medium containing 20% FBS plus pen-strep-glutamine. 24 hours after seeding, the cells were treated with control hFc, the anti-MET parental bivalent monospecific antibody H4H13312P2, or the MET×MET bispecific antibodies (H4H14634D, H4H14635D, H4H14636D, H4H14637D, H4H14638D, H4H14639D, H4H14640D, H4H14641 D) for 18 hrs. Cell lysates were then prepared and analyzed by western blotting. Immunoblots were probed for MET and tubulin. The MET protein expression level was quantified and normalized relative to the tubulin loading control. The results are presented in Table 15 and FIG. 10, panel B.

TABLE 15
Relative Level of MET Protein

| Molecule | Relative MET Level |
|---|---|
| Control (hFc) | 1 |
| H4H13312P2 | 0.62 |
| H4H14634D | 0.45 |
| H4H14635D | 0.27 |
| H4H14636D | 0.50 |
| H4H14637D | 0.49 |
| H4H14638D | 0.35 |
| H4H14639D | 0.27 |
| H4H14640D | 0.18 |
| H4H14641D | 0.31 |

SNU5 cancer cells were treated with control antibody or MET×MET bispecific antibody or monovalent MET antibody at 10 μg/ml for 18 hrs as described above. MET expression (FIG. 10, panels A and B), and pathway activation (i.e., pMET and pERK; panel A) were determined by immunoblotting with the indicated antibodies. The immunoblots are shown in FIG. 10.

Treatment of SNU5 cells with MET×MET bispecific antibodies induced more potent degradation of MET than treatment with the bivalent monospecific anti-MET antibody (H4H13312P2) (FIG. 10, panel B), monovalent MET antibody or control hFc. Treatment of SNU5 cells with the MET×MET bispecific antibody inhibited downstream effectors of the MET pathway. Similar results were obtained for the MET amplified non-small cell lung cancer adenocarcinoma cell line NCI-H1993 (Kubo et al., “MET gene amplification or EGFR mutation activate MET in lung cancers untreated with EGFR tyrosine kinase inhibitors,” Int. J. Cancer 2009 Apr. 15; 124(8): 1778-1784).

### Example 13. A MET×MET Bispecific Antibody Induces MET Degradation, Inhibits Pathway Activity, and Inhibits Tumor Growth More Potently than Monospecific Antibodies in EBC-1 Cells

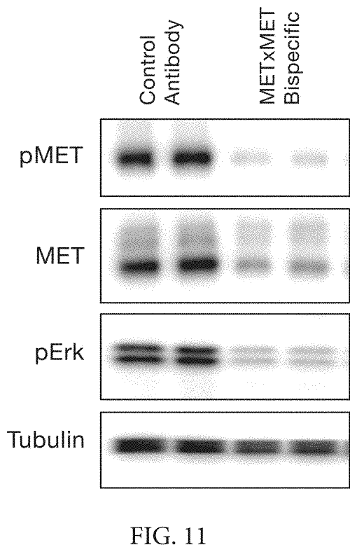

MET-amplified human lung squamous cell carcinoma EBC-1 cells (Lutterbach et al., “Lung cancer cell lines harboring MET gene amplification are dependent on Met for growth and survival,” Cancer Res. 2007 Mar. 1; 67(5):2081-8) were treated with a control antibody or 10 μg/ml of a MET×MET bispecific antibody for 18 hrs as described above. MET expression and MET pathway activation ascertained by pMET and pErk expression were determined by immunoblotting with the indicated antibodies. The immunoblots are shown in FIG. 11.

Treatment of EBC-1 cells, which harbor MET gene amplification, with MET×MET bispecific antibodies induced more potent degradation of MET than treatment with the control antibody. Treatment of EBC-1 cells with the MET×MET bispecific antibody inhibited downstream effectors of the MET pathway.

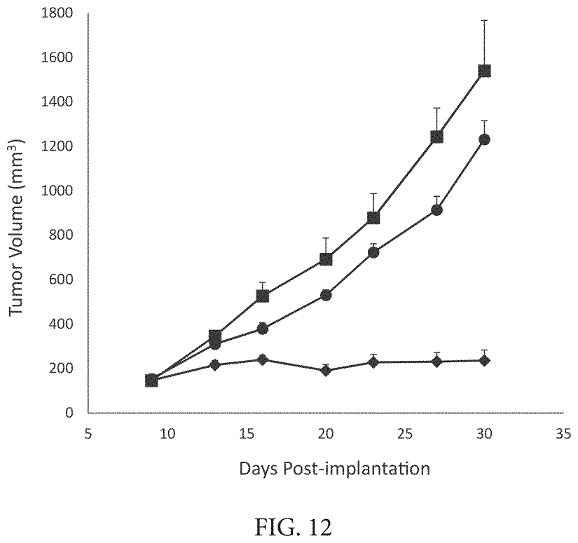

In another experiment, 5 million EBC-1 cells were implanted subcutaneously into the flank of C.B.-17 SCID mice. Once the tumor volumes reached approximately 150 mm³, mice were randomized into groups of 6 and were treated twice a week with a control antibody at 25 mg/kg or the MET×MET bispecific antibody H4H14639D at 25 mg/kg. Tumor growth was monitored for 30 days post-implantation and tumor volume (mm³) was measured for each experimental group over time. The results are depicted in Table 16 and FIG. 12, which shows that the MET×MET bispecific antibody significantly inhibits the growth of EBC-1 tumors.

TABLE 16
Relative EBC-1 Tumor Growths

| | Tumor Growth (mm3) |
|---|---|
| | form the start of treatment |
| Treatment | (mean ± SEM) |
| 25 mg/kg Control | 1394 ± 226 |
| 25 mg/kg H4H14639D | 89 ± 47 |

### Example 14. A MET×MET Bispecific Antibody Inhibits In Vitro Growth of Hs746T Gastric Cancer Cells More Potently than Monospecific Antibodies

The effect of a MET×MET bispecific antibody on the growth of human gastric carcinoma cells was assessed in vitro. 2,500 Hs746T human gastric carcinoma cells (H. Smith, J. Nat'l. Cancer Inst. 62(2): 225-230, 1979) were seeded in a 96 well plate and cultured in Modified Dulbecco's Media supplemented with 10% FBS. The cells were treated with (1) individual bivalent monospecific anti-MET antibodies (H4H13306P2 or H4H13312P2) at 5 μg/ml, (2) a combination of the two bivalent monospecific anti-MET parental antibodies (H4H13306P2 and H4H13312P2) at 2.5 μg/ml each, or (3) the bispecific antibody containing one binding arm from H4H13306P2 and the other binding arm from H4H13312P2 (H4H14639D) at 5 μg/ml. The cells were subsequently incubated with 5% CO₂ at 37° C. After 5 days, relative cell growth was determined by measuring the reduction of the indicator dye, ALAMAR BLUE® (ThermoFischer Scientific, Waltham, Mass.), to its highly fluorescent form in a SPECTRAMAX® M3 plate reader (Molecular Devices, Sunnyvale, Calif.). Increasing fluorescence correlates with cell growth. Table 17 depicts the relative Hs746T cell growth for each antibody treatment normalized to control (no treatment) Hs746T cell growth. The bispecific antibody, H4H14639D, inhibits the proliferation of Hs746T cells more potently than its parental monospecific antibodies individually or in combination.

TABLE 17
Normalized Hs746T Cell Growth

| | Relative Cell Growth | Standard |
|---|---|---|
| | (n = 3) | Deviation |
| Control | 1 | 0.133497801 |
| H4H14639D | 0.647408139 | 0.019090432 |
| H4H13306P2 | 1.623312821 | 0.189647479 |
| H4H13312P2 | 0.852680493 | 0.01728527 |
| H4H13306P2 + | 1.767720125 | 0.077445717 |
| H4H13312P2 | | |

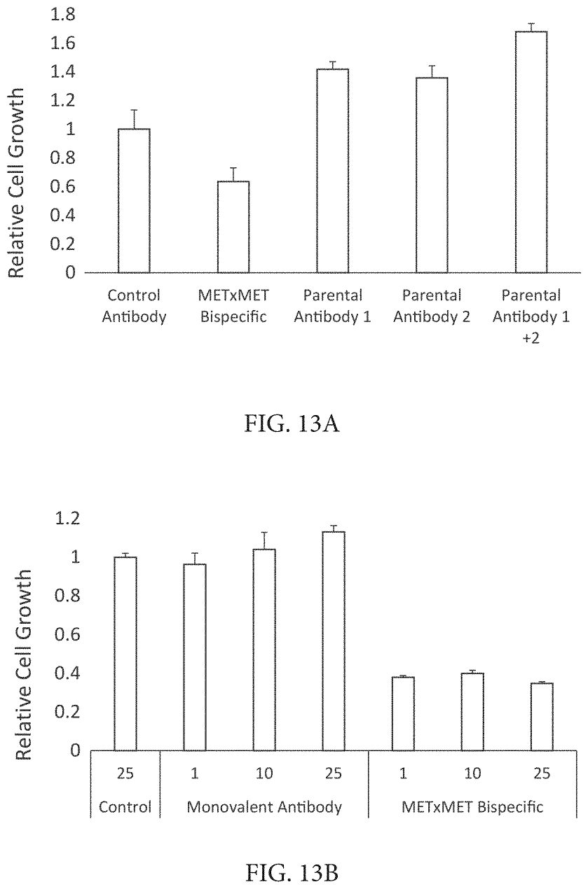

Hs746T gastric cancer cells were treated with control antibody, the MET×MET bispecific antibody H4H14639D, the anti-MET parental antibody H4H13306P2, the anti-MET parental antibody H4H13312P2, and the combination of parental antibodies 1 and 2, each antibody at 2 μg/ml. Cell growth was determined after 5 days by reduction of ALAMAR BLUE® reagent (FIG. 13, panel A). The MET×MET bispecific antibody inhibited cell growth relative to the parental antibodies alone or combined, and inhibited MET pathway activation more effectively than its parental antibodies in Hs746T gastric cancer cells.

Hs746T gastric cancer cells in 96 well plates were treated with 25 μg/mL control antibody, 1 μg/mL, 10 μg/mL or 25 μg/mL monovalent MET antibody, or 1 μg/mL, 10 μg/mL or 25 μg/mL MET×MET bispecific antibody. Hs746T gastric cancer cell growth was determined after 5 days by reduction of ALAMARBLUE® reagent (FIG. 13, panel B). MET×MET bispecific antibody potently inhibits growth of MET-amplified cells.

### Example 15. A MET×MET Bispecific Antibody does not Induce Growth of NCI-H596 Lung Cancer Cells In Vitro

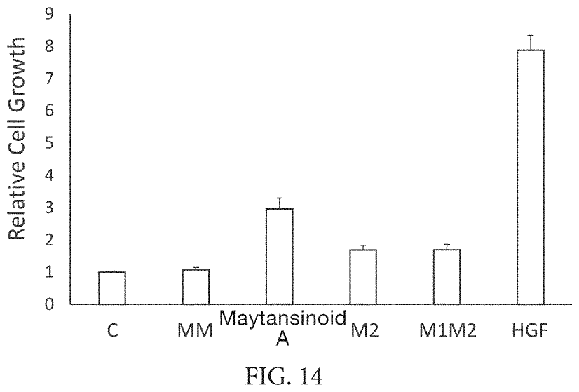

The effect of a MET×MET bispecific antibody on the growth of human non-small cell lung cancer (NSCLC) cells (NCI-H596) was assessed in vitro. 10,000 NCI-H596 lung adenosquamous carcinoma cells (Nair et al., J. Nat'l. Cancer Inst. 86(5): 378-383, 1994) were seeded in 96 well plates on a layer of 0.66% agar in media supplemented with 1% fetal bovine serum (FBS). The cells were cultured in RPMI 1640 media supplemented with 1% FBS with 0.3% agarose. The cells were treated with (1) individual parental bivalent monospecific anti-MET antibodies (H4H13306P2 or H4H13312P2) at 5 μg/ml, (2) a combination of the two parental bivalent monospecific anti-MET antibodies (H4H13306P2 and H4H13312P2) at 2.5 μg/ml each, (3) a bispecific antibody containing one binding arm from H4H13306P2 and the other binding arm from H4H13312P2 (H4H14639D) at 5 μg/ml, or (4) 100 ng/mL of hepatocyte growth factor (HGF). The cells were subsequently incubated with 5% CO₂ at 37° C. After two weeks, relative cell growth was determined by measuring the reduction of the indicator dye, ALAMAR BLUE® (Thermo Fischer Scientific, Waltham, Mass.), to its highly fluorescent form in a SPECTRAMAX® M3 plate reader (Molecular Devices, Sunnyvale, Calif.). Increasing fluorescence correlates with cell growth. Table 18 and FIG. 14 depict the relative NCI-H596 cell growth for each antibody treatment normalized to control (no treatment) NCI-H596 cell growth. Treatment of NCI-H596 lung cancer cells with HGF resulted in potent induction of growth in soft agar. The MET×MET (MM in FIG. 14) bispecific antibody H4H14639D did not significantly alter growth relative to control treated cells. Modest induction of cell growth was observed with each parental bivalent monospecific antibody H4H13306P2 (M1) or H4H13312P2 (M2) individually, or combined (H4H13306P2 and H4H13312P2) (M1M2).

TABLE 18
Normalized NCI-H596 Cell Growth

| | Relative Cell Growth | Standard |
|---|---|---|
| | (n = 3) | Deviation |
| Control | 1 | 0.030074808 |
| H4H14639D | 1.070339237 | 0.075103746 |
| H4H13306P2 | 2.9593578 | 0.337877264 |
| H4H13312P2 | 1.686580346 | 0.145670753 |
| H4H13306P2 + | 1.693724668 | 0.168651046 |
| H4H13312P2 | | |
| HGF | 7.87655937 | 0.46057617 |

### Example 16. A MET×MET Bispecific Antibody Inhibits In Vitro Growth of SNU5 Gastric Cancer Cells More Potently than Monospecific Antibodies

The effect of a MET×MET bispecific antibody on the growth of human gastric carcinoma cells was assessed in vitro. 2,500 SNU5 human gastric carcinoma cells (Ku and Park, Cancer Res. Treat. 37(1): 1-19, 2005) were seeded in a 96 well plate and cultured in Iscove's Modified Dulbecco's Media supplemented with 20% FBS. The cells were treated with (1) individual bivalent monospecific anti-MET antibodies (H4H13306P2 or H4H13312P2) at 5 μg/ml, (2) a combination of the two bivalent monospecific anti-MET antibodies (H4H13306P2 and H4H13312P2) at 2.5 μg/ml each, or (3) a bispecific antibody containing one binding arm from H4H13306P2 and the other binding arm from H4H13312P2 (H4H14639D) at 5 μg/ml. The cells were subsequently incubated with 5% CO₂ at 37° C. After 5 days, relative cell growth was determined by measuring the reduction of the indicator dye, ALAMAR BLUE® (Thermo Fischer Scientific, Waltham, Mass.), to its highly fluorescent form in a SPECTRAMAX® M3 plate reader (Molecular Devices, Sunnyvale, Calif.). Increasing fluorescence correlates with cell growth. Table 19 depicts the relative SNU5 cell growth for each antibody treatment normalized to control (no treatment) SNU5 cell growth. The bispecific antibody, H4H14639D, inhibits the proliferation of SNU5 cells more potently than its parental monospecific antibodies.

TABLE 19
Normalized SNU5 Cell Growth

| | Relative Cell Growth | Standard |
|---|---|---|
| | (n = 3) | Deviation |
| Control | 1 | 0.070814765 |
| H4H14639D | 0.271100069 | 0.01324024 |
| H4H13306P2 | 0.766317547 | 0.061930288 |
| H4H13312P2 | 0.431990234 | 0.033183065 |
| H4H13306P2 + | 0.331287005 | 0.012042949 |
| H4H13312P2 | | |

### Example 17. A MET×MET Bispecific Antibody Induces Regression of Hs746T Tumor Xenograft

The effect of a MET×MET bispecific antibody on a human gastric carcinoma tumor in an immunocompromised mouse model was assessed. Three million Hs746T human gastric carcinoma cells were implanted subcutaneously into the flank of CB-17 SCID mice (Bancroft et al., J. Immunol. 137(1): 4-9, 1986). Once the tumor volumes reached approximately 200 mm³, the mice were randomized into groups of six and were treated twice per week with a control antibody at 25 mg/kg or with a MET×MET bispecific antibody (H4H14639D) at 25 mg/kg. Tumor growth was monitored for 16 days post-implantation for the control group, when the control-treated tumors reached protocol size limits. Tumor growth was monitored for 30 days post-implantation for the H4H14639-treated group.

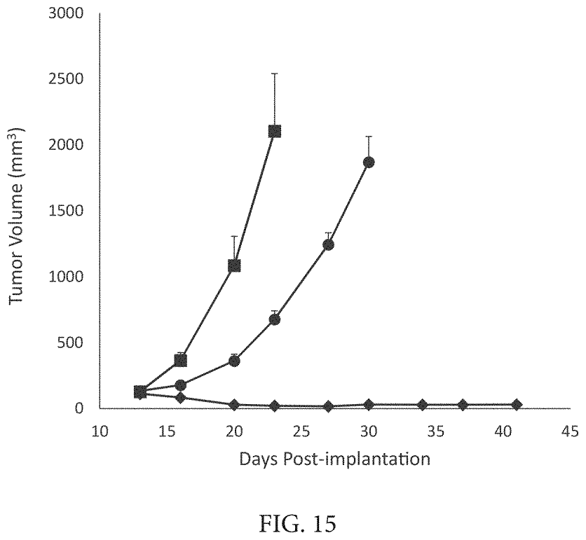

Treatment of tumors with the MET×MET bispecific antibody induced regression of tumor size over 21 days relative to the beginning of treatment. The control-treated tumors showed a mean increase in volume of about 12-fold over 16 days of growth (Table 20). Tumor volume over time, which shows Hs746T tumor regression due to the MET×MET bispecific antibody, is shown in FIG. 15.

TABLE 20
Hs746T Gastric Tumor Growth

| | Tumor growth (mm³) |
|---|---|
| | from the start of treatment |
| Antibody (mg/kg) | (mean ± SEM) |
| Control (10) | 1164 ± 138 |
| H4H14639D (25) | −215 ± 8.3 |

### Example 18. A MET×MET Bispecific Antibody Induces Regression of SNU5 Tumor Xenograft

The effect of a MET×MET bispecific antibody on a human gastric carcinoma tumor in an immunocompromised mouse model was assessed. Ten million SNU5 human gastric carcinoma cells were implanted subcutaneously into the flank of CB-17 SCID mice. Once the tumor volumes reached approximately 500 mm³, the mice were randomized into groups of five and were treated twice per week with a control antibody at 10 mg/kg or with a MET×MET bispecific antibody (H4H14639D) at either 1 mg/kg or 10 mg/kg. Tumor growth was monitored for 81 days post-implantation when the control-treated tumors reached protocol size limits.

The tumors of mice treated with 1 mg/kg or 10 mg/kg of the MET×MET antibody demonstrated a mean reduction in size of about 95% or 98%, respectively. The control-treated tumors showed a mean increase in volume of about 12-fold from the start of treatment (Table 21).

TABLE 21
SNU5 Gastric Tumor Growth

| | Tumor growth (mm³) |
|---|---|
| | from the start of treatment |
| Antibody (mg/kg) | (mean ± SEM) |
| Control (10) | 1123 ± 194 |
| H4H14639D (1) | −477 ± 43 |
| H4H14639D (10) | −492 ± 18 |

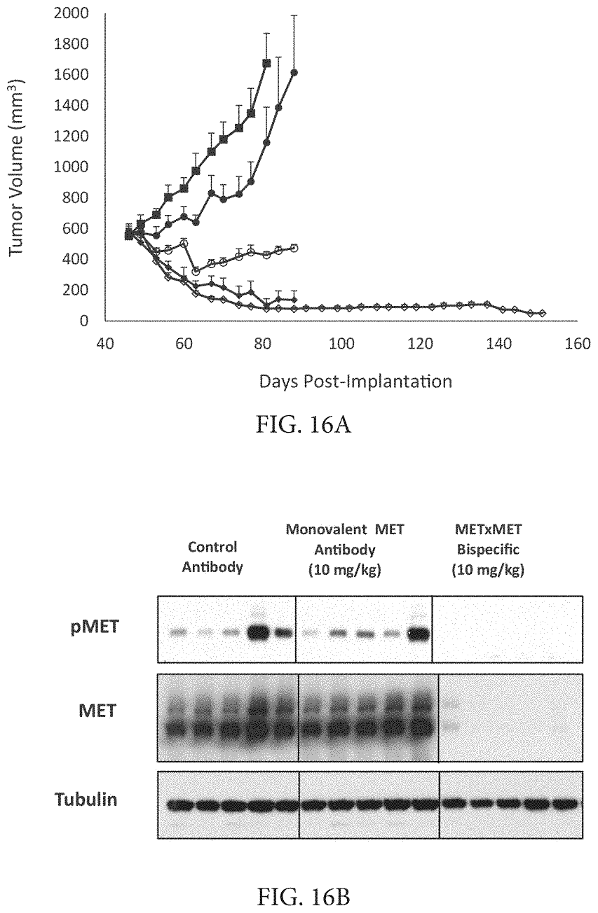

Subcutaneously implanted SNU5 tumors were treated twice weekly with control antibody, monovalent MET antibody at 1 mg/kg or 10 mg/kg, or MET×MET bispecific antibody at 1 mg/kg or 10 mg/kg. Potent and sustained regression of MET-amplified SNU5 tumors (i.e., reduction in tumor volume) was observed over time in those mice treated with MET×MET bispecific antibody (FIG. 16, panel A). Protein was extracted from the end-of-study tumors and MET expression and pathway activation as indicated by MET phosphorylation (pMET expression) were determined by immunoblotting. The MET×MET treated mice (tumors) showed reduction in MET and pMET expression relative to the controls (FIG. 16, panel B). The MET×MET bispecific antibody is a potent inhibitor of tumors harboring MET amplification.

### Example 19. A MET×MET Bispecific Antibody Induces Regression of U87-MG Tumor Xenograft

The effect of a MET×MET bispecific antibody on a human glioblastoma tumor in an immunocompromised mouse model was assessed. Five million U87-MG human glioblastoma cells (Vordermark and Brown, Int. J. Radiation Biol. 56(4): 1184-1193, 2003) were implanted subcutaneously into the flank of CB-17 SCID mice. U87-MG glioblastoma xenograft models are driven by autocrine HGF signaling. Once the tumor volumes reached approximately 100 mm³, the mice were randomized into groups of six and were treated with a control antibody or the MET×MET bispecific antibody (H4H14639D). 25 mg/kg of antibody (control or MET×MET) was administered to each mouse twice per week. Tumor growth was monitored for 29 days post-implantation when the control-treated tumors reached protocol size limits.

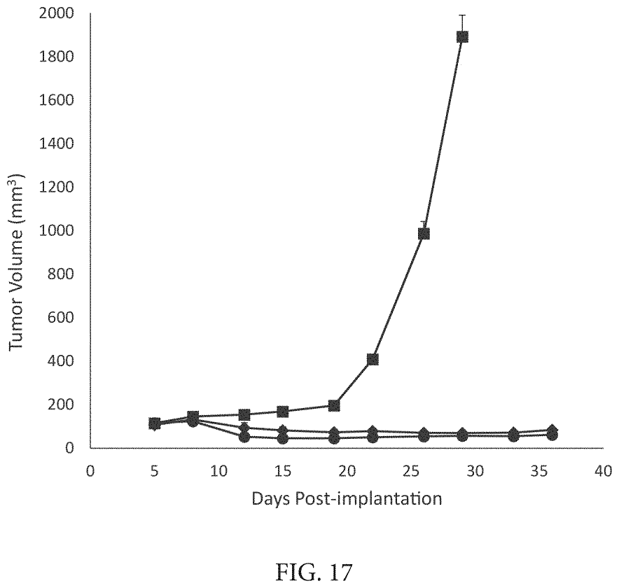

The tumors of mice treated with the MET×MET antibody demonstrated a mean reduction in size of about 38%, whereas the control-treated tumors showed a mean increase in volume of about 19-fold over 29 days of growth (Table 22). Tumor volume over time, which shows U87-MG tumor regression due to the MET×MET bispecific antibody, is shown in FIG. 17.

TABLE 22
Glioblastoma Tumor Growth

| | Tumor growth (mm3) |
|---|---|
| | from the start of treatment |
| Antibody (mg/kg) | (mean ± SEM) |
| Control (25) | 1777 ± 98 |
| H4H14639D (25) | −38 ± 18 |

### Example 20. A MET×MET Bispecific Antibody Inhibits Growth of U118-MG Tumor Xenograft

The effect of a MET×MET bispecific antibody on a human glioblastoma tumor in an immunocompromised mouse model was assessed. U118-MG glioblastoma xenograft models are driven by autocrine HGF signaling. Five million U118-MG human glioblastoma cells (Olopade et al., Cancer Research 52: 2523-2529, 1992) were implanted subcutaneously into the flank of CB-17 SCID mice. Once the tumor volumes reached approximately 100 mm³, the mice were randomized into groups of six and were treated with a control antibody or the MET×MET bispecific antibody (H4H14639D). 25 mg/kg of antibody (control or MET×MET) was administered to each mouse twice per week. Tumor growth was monitored for 72 days post-implantation.

The MET antibody inhibited tumor growth by 99% over the 72 day period (Table 23).

TABLE 23
Glioblastoma Tumor Growth

| | Tumor growth (mm3) | % Decrease in |
|---|---|---|
| | from the start of treatment | tumor growth |
| Antibody (mg/kg) | (mean ± SEM) | versus control |
| Control (25) | 1228 ± 123 | — |
| H4H14639D (25) | 11 ± 18 | 99.1 |

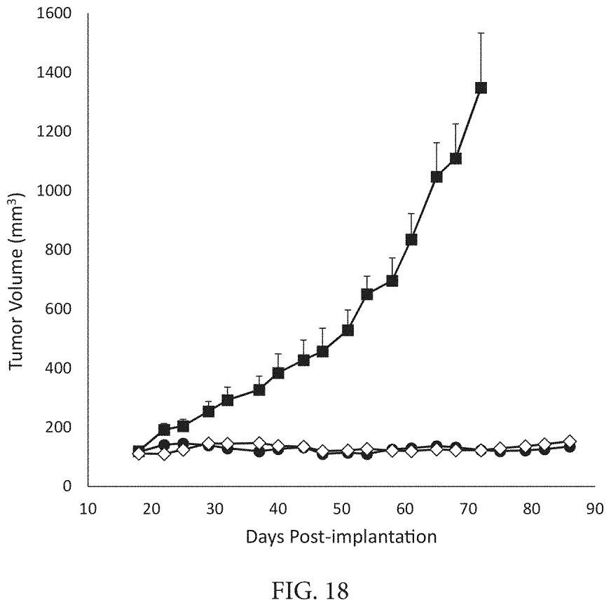

In another experiment, subcutaneously implanted U118-MG glioblastoma tumors in mice were treated twice weekly with 25 mg/kg control antibody, monovalent MET antibody or MET×MET bispecific antibody. Tumor volume (mm³) was measured for each experimental group over time. The results are depicted in FIG. 18, which shows the MET×MET bispecific antibody inhibits growth of U118-MG tumors.

### Example 21: Maytansinoid Synthesis

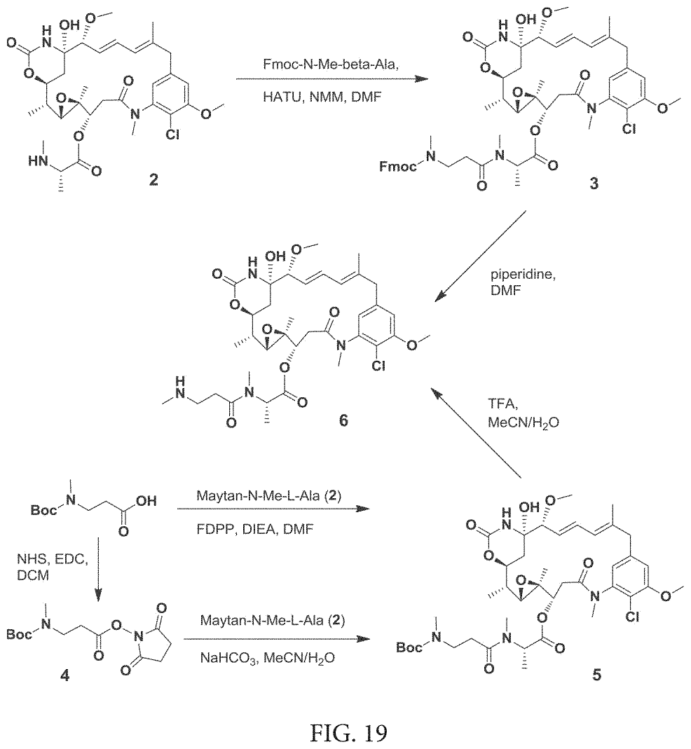

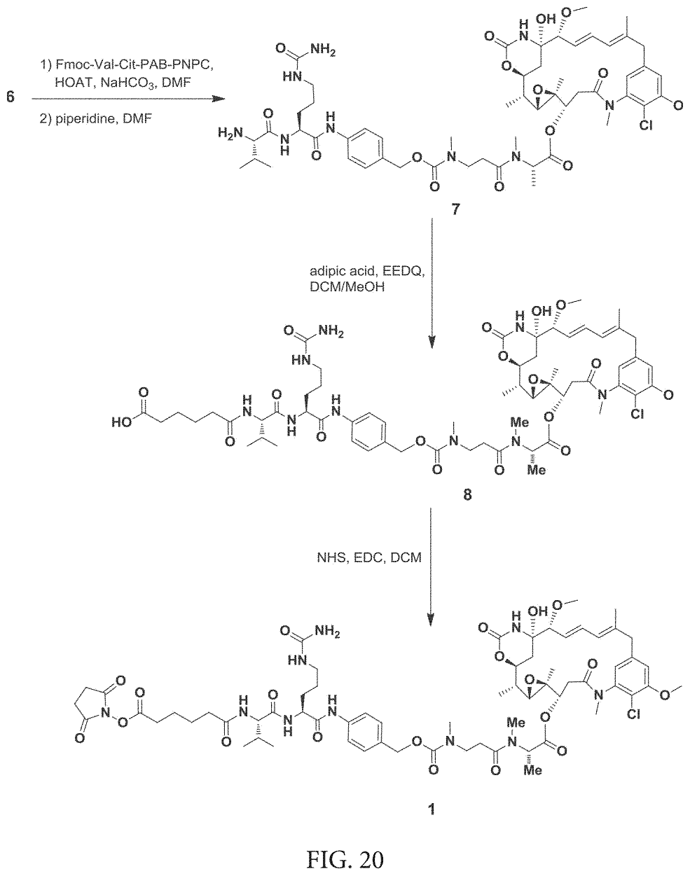

Maytansin-3-N-methyl-L-alanine-N-Me-beta-alanine-carbamyl-(p-amino)benzyl-citrulline-valine-adipoyl-succinate (Compound 1 in FIG. 20) was synthesized from compound 2 (FIG. 19) as described below.

Maytansin-3-N-methyl-L-alanine-Fmoc-N-Me-beta-alanine (Compound 3, FIG. 19). Des-acetyl-maytansine (Compound 2, FIG. 19, 0.433 g, 0.666 mmol), Fmoc-N-Me-beta-Ala (0.434 g, 1.33 mmol), and HATU (0.757 g, 1.99 mmol) were weighed to a dry flask, dissolved in anhydrous DMF (9 mL), and treated with 4-methylmorpholine (0.300 mL, 2.73 mmol). The flask was sealed with a rubber septum, purged with argon, and the reaction stirred at ambient temperature. After 3 days the mixture was evaporated to an oil, dissolved in acetonitrile and water, and purified by flash chromatography on a 275 g C18 silica column (30-90% acetonitrile in water over 20 min, 0.05% acetic acid in both phases). Lyophilization of the product fractions gave the title compound as a white solid. The crude was purified on an 80 g silica gel column (EtOAc—5:5:1 EtOAc:DCM:MeOH over 17 min). The pure fractions were combined, evaporated, and dried in vacuo overnight giving the title compound as a white solid (0.424 g, 66%). MS (ESI, pos.): calc'd for C₅₁H₆₁ClN₄O₁₂, 956.4; found 956.9 (M+H), 979.0 (M+Na), 939.0 (M−H₂O+H).

N-tert-Butoxycarbonyl-N-methyl-beta-alanine succinate ester (Compound 4, FIG. 19). The title compound was prepared from commercial Boc-N-Me-beta-Ala-OH by a method well known in the art (cf.—Widdison et al., J. Med. Chem., 2006, 49 (14), 4401). ¹H NMR (300 MHz, CDCl₃): δ 3.62 (bm, 2H), 2.88 (m, 9H), 1.47 (s, 9H).

Maytansin-3-N-methyl-L-alanine-Boc-N-Me-beta-alanine (Compound 5, FIG. 19). Method A: The product of the preceding step (Compound 4, FIG. 19, 0.453 g, 1.51 mmol) and des-acetyl-maytansine (Compound 2, FIG. 19, 0.304 g, 0.468 mmol) were dissolved in 3:1 acetonitrile:water (8 mL), treated with 1M aqueous NaHCO₃ (0.5 mL), and stirred at ambient temperature for 18 hours. When the reaction was complete as determined by TLC, it was then stirred with brine for 10 min and extracted thrice with ethyl acetate (EtOAc). The combined organic layers were then dried over Na₂SO₄, filtered, and the filtrate concentrated and dried in vacuo to a gold syrup that was purified by flash column chromatography on a 20 g silica gel cartridge (0-10% MeOH in EtOAc over 15 min) giving the title compound as a white solid (0.084 g, 43%). MS (ESI, pos.): calc'd for C₄₁H₅₉ClN₄O₁₂, 834.4; found 835.2 (M+H), 857.2 (M+Na), 817.4 (M−H₂O+H).

Method B: Boc-N-Me-beta-Ala-OH (0.294 g, 1.45 mmol) was dissolved in anhydrous DMF (5 mL), treated with pentafluorophenyl diphenylphosphinate (FDPP, 0.555 g, 1.44 mmol), and the reaction stirred at ambient temperature for 30 min. The mixture was then transferred to a larger flask containing a mixture of des-acetyl-maytansine (Compound 2, FIG. 19, 0.462 g, 0.711 mmol) and diisopropylethylamine (DIEA, 0.250 mL, 1.44 mmol) in anhydrous DMF (7 mL), the flask sealed with a rubber septum, purged with argon, and reaction stirred again at ambient temperature. After 24 hours the reaction was concentrated in vacuo to an oil, dissolved in ethyl acetate (EtOAc, 2 mL), and purified on a 40 g silica gel cartridge (EtOAc—5:5:1 EtOAc/DCM/MeOH over 15 min), giving the title compound as a pale yellow solid (0.468 g, 79%). MS (ESI, pos.): calc'd for C₄₁H₅₉ClN₄O₁₂, 834.4; found 857.2 (M+Na), 817.2 (M−H₂O+H).

Maytansin-3-N-methyl-L-alanine-N-Me-beta-alanine (Compound 6, FIG. 19). Method A: Maytansin-N-Me-L-Ala-Boc-N-Me-beta-Ala (Compound 5, FIG. 19, 0.464 g, 0.555 mmol) was dissolved in a 3:1:1 mixture of acetonitrile/water/trifluoroacetic acid (7 mL), the flask sealed with a rubber septum, purged with argon, and the reaction stirred at ambient temperature for 24 hours, then capped and stored at −20° C. for 3 days. The crude reaction mixture was warmed to ambient temperature for 2 hours, briefly concentrated in vacuo, purified on a 100 g C18 RediSep Gold column (20-80% acetonitrile in water over 25 min, 0.1% TFA in both solvents), and the combined pure fractions were partially evaporated at ambient temperature, frozen in a dry ice bath, and lyophilized to give the title compound as a pale yellow solid (0.295 g, 63%). MS (ESI, pos.): calc'd for C₃₆H₅₁ClN₄O₁₀, 734.3; found 735.7 (M+H), 1471.3 (2M+H).

Method B: Maytansin-N-Me-L-Ala-Fmoc-beta-Ala (Compound 3, FIG. 19, 0.422 g, 0.441 mmol) was dissolved in 5% piperidine in DMF (6.00 mL, 3.04 mmol), the reaction flask sealed with a rubber septum, purged with argon, and the mixture stirred at ambient temperature. After 3 hours the reaction was complete by LCMS, so it was concentrated in vacuo, sealed, and stored at −20° C. overnight. The crude product was warmed to ambient temperature, treated with acetonitrile and 10% aq. acetic acid (3 mL each), and purified by flash chromatography on a 275 g C18 silica column (10-90% acetonitrile in water over 20 min, 0.05% acetic acid in both solvents). Lyophilization of the product fractions gave the title compound as a white solid. The solid was triturated thrice with dry diethyl ether, filtered, the solids washed off the frit with DCM, and the filtrate evaporated and dried in vacuo giving the title compound as a white solid (0.311 g, 89%). MS (ESI, pos.): calc'd for C₃₆H₅₁ ClN₄O₁₀, 734.3; found 735.0 (M+H).

Maytansin-3-N-methyl-L-alanine-N-Me-beta-alanine-carbamyl-(p-amino)benzyl-citrulline-valine-Fmoc (Compound 7, FIG. 20). Step 1: The product of the preceding step (Compound 6, FIG. 19, 0.310 g, 0.390 mmol), 1-hydroxy-7-azabenzotriazole (HOAT, 0.164 g, 1.20 mmol), sodium bicarbonate (0.138 g, 1.64 mmol), and Fmoc-valine-citrulline-(p-amino)benzyl-(p-nitrophenyl)carbonate (0.595 g, 0.776 mmol, prepared by method known in the art, cf.—Gangwar et al., U.S. Pat. No. 7,714,016 B2) were dissolved in anhydrous DMF (10 mL), the reaction flask sealed with a rubber septum, purged with argon, and the mixture stirred at ambient temperature. After 24 hours the reaction was partially evaporated in vacuo to ca. 2-3 mL, treated with 10% aq. acetic acid and water (ca. 1 mL each), dissolved in acetonitrile (ca. 6 mL), and purified by flash chromatography on a 275 g C18 silica column (30-90% acetonitrile in water over 20 min, 0.05% acetic acid in both solvents). Partial evaporation, freezing, and lyophilization gave the title compound as a white solid (0.362 g, 68%). MS (ESI, pos.): calc'd for C₇₀H₈₈ClN₉O₁₇, 1361.6; found 1362.1 (M+H), 1384.1 (M+Na), 1344.1 (M−H₂O+H).

Step 2: The product of the preceding step (0.360 g, 0.264 mmol) was dissolved in 5% piperidine in DMF (7 mL), the reaction flask sealed with a rubber septum, purged with argon, and the mixture stirred at ambient temperature. After 3 hours the reaction was evaporated in vacuo, the residue treated with 10% aq. acetic acid (2 mL), dissolved in acetonitrile (4 mL), and purified by flash chromatography on a 275 g C18 silica column (10-70% acetonitrile in water over 20 min, 0.05% acetic acid in both solvents). The pure fractions were combined, stored at −20° C. overnight, partially evaporated in vacuo at 25-30° C., frozen on dry ice, and lyophilized for 6 days giving the title compound as a pale yellow solid (0.303 g, 95%). MS (ESI, pos.): calc'd for C₁₅H₇₈ClN₉O₁₅, 1139.5; found 1140.1 (M+H), 1162.0 (M+Na).

Maytansin-3-N-methyl-L-alanine-N-Me-beta-alanine-carbamyl-(p-amino)benzyl-citrulline-valine-adipic acid (Compound 8, FIG. 20). The product of the preceding step (Compound 7, FIG. 20, 0.205 g, 0.171 mmol), adipic acid (0.258 g, 1.77 mmol), and 2-ethoxy-1-ethoxycarbonyl-1,2-dihydroquinoline (EEDQ, 0.215 g, 0.869 mmol) were dissolved in dry DCM (10 mL) and anhydrous methanol (5 mL), the reaction flask was sealed with a rubber septum, purged with argon, and the mixture stirred at ambient temperature. After 21 hours the reaction was evaporated in vacuo, the residue dissolved in a few mL of acetonitrile/water, and purified by flash chromatography on a 150 g C18 silica column (20-80% acetonitrile in water over 17 min, 0.05% acetic acid in both solvents). Partial evaporation, freezing, and lyophilization of the pure fractions for 18 hours gave the title compound as a white solid (0.140 g, 65%). MS (ESI, pos.): calc'd for C₆₁H₈₆ClN₉O₁₈, 1267.6; found 1268.9 (M+H), 1290.9 (M+Na).

Maytansin-3-N-methyl-L-alanine-N-Me-beta-alanine-carbamyl-(p-amino)benzyl-citrulline-valine-adipoyl-succinate (Compound 1, FIG. 20). The product of the preceding step (Compound 8, FIG. 20, 0.061 g, 0.048 mmol), N-hydroxysuccinimide (0.063 g, 0.55 mmol), and N-(3-Dimethylaminopropyl)-N′-ethylcarbodiimide hydrochloride (EDC-HCl, 0.071 g, 0.37 mmol) were dissolved in dry DCM (7 mL), the reaction flask sealed with a rubber septum, purged with argon, and the mixture stirred at ambient temperature. After 5 days the reaction was evaporated in vacuo, the residue dissolved in a few mL of acetonitrile/water, and purified by flash chromatography on a 100 g C18 silica column (30-90% acetonitrile in water over 15 min, 0.05% acetic acid in both solvents). Partial evaporation, freezing, and lyophilization of the cleanest product fractions for 18 hours gave the title compound as a white solid (0.044 g, 67%). MS (ESI, pos.): calc'd for C₆₅H₈₉ClN₁₀O₂₀, 1364.6; found 1365.7 (M+H), 1387.7 (M+Na), 1347.7 (M−H₂O+H). ¹H-NMR (500 MHz; CDCl₃): δ 7.56 (d, J=8.3 Hz, 2H), 7.20 (d, J=8.7 Hz, 1H), 6.80 (s, 1H), 6.71 (m, 1H), 6.62 (d, J=10.0 Hz, 1H), 6.39 (dd, J=15.1, 11.3 Hz, 1H), 5.68 (dd, J=15.3, 9.1 Hz, 1H), 5.38-5.32 (m, 1H), 5.03 (t, J=15.1 Hz, 1H), 4.88 (d, J=12.3 Hz, 1H), 4.73 (d, J=11.3 Hz, 1H), 4.61 (dd, J=9.1, 3.6 Hz, 1H), 4.26 (d, J=7.0 Hz, 1H), 4.17 (t, J=7.1 Hz, 1H), 3.95 (s, 3H), 3.61 (d, J=11.7 Hz, 1H), 3.57 (d, J=12.4 Hz, 1H), 3.46 (d, J=9.1 Hz, 2H), 3.33 (s, 3H), 3.27 (t, J=6.9 Hz, 1H), 3.17-3.07 (m, 5H), 2.97 (dd, J=16.6, 9.9 Hz, 1H), 2.88 (d, J=11.7 Hz, 3H), 2.84 (s, 4H), 2.77 (s, 2H), 2.66 (s, 2H), 2.62 (t, J=4.8 Hz, 2H), 2.56 (d, J=13.1 Hz, 1H), 2.32 (t, J=6.6 Hz, 2H), 2.15 (d, J=14.0 Hz, 1H), 2.10 (q, J=6.8 Hz, 1H), 1.92 (s, 4H), 1.75 (m, 5H), 1.61 (s, 3H), 1.52 (s, 3H), 1.27 (d, J=6.3 Hz, 3H), 1.22 (dt, J=12.7, 6.3 Hz, 6H), 0.95 (t, J=5.9 Hz, 7H), 0.78 (s, 3H).

DM1 was synthesized as a single diastereomer based on the procedures described in WO 2015/031396 (e.g., Example 2, paragraph [00106]), incorporated herein by reference in its entirety.

### Example 22. Antibody Conjugation and Characterization of Conjugates

Antibody Conjugation

The antibodies (H4H14639D, H4H13312P, H4H14635D, and isotype control; 10-20 mg/ml) in 50 mM HEPES, 150 mM NaCl, pH 8.0, and 10-15% (v/v) DMA were conjugated with a 5-6 fold excess of SMCC-DM1 diastereomer prepared as described in Example 21 (Maytansinoid A) or maytansin-3-N-methyl-L-alanine-N-Me-beta-alanine-carbamyl-(p-amino)benzyl-citrulline-valine-adipoyl-succinate (Compound 1, FIG. 20) (Maytansinoid B) for 2 hours at ambient temperature. The conjugates were purified by size exclusion chromatography or extensive ultrafiltration and sterile filtered. Protein concentrations were determined by UV spectral analysis. Size-exclusion HPLC established that all conjugates used were >90% monomeric, and RP-HPLC established that there was <1% unconjugated linker payload. All conjugated antibodies were analyzed by UV for linker payload loading values according to Hamblett et al. (American Association for Cancer Research. 2004 Oct. 15; 10(20):7063-70) and/or by mass difference, native versus conjugated. Payload to antibody ratios are reported in Table 24.

TABLE 24
Percent Yield and Payload to Antibody Ratios
for Each of the Antibody Drug Conjugates

| Antibody | Yield (%) | DAR (MS) | DAR (UV) |
|---|---|---|---|
| H4H14639D-maytansinoid A | 60 | 3.8 | 3.7 |
| H4H14639D-maytasinoid B | 50 | 2.4 | 2.4 |
| H4H13312P-maytansinoid A | 60 | 4.1 | 4.1 |
| H4H13312P-maytansinoid B | 50 | 2.3 | 2.5 |
| Isotype Control | 70 | 2.3 | 2.5 |
| REGN1945-maytansinoid B | | | |
| Isotype Control | 80 | 3.7 | 3.7 |
| REGN1945-maytansinoid A | | | |

### Characterization of Conjugates by Liquid Chromatography-Mass Spectrometry

To determine the loading of the linker-payloads on the antibody, the conjugates were deglycosylated, and analyzed by LC-MS.

For the assay, 50 μg of the conjugate was diluted with milli-Q water to a final concentration of 1 mg/mL. Ten μL of PNGase F solution [PNGase F solution was prepared by adding 1504 of PNGase F stock (New England Biolabs, Cat #P0704L) and 8504 of milli-Q water and mixed well] was added to the diluted conjugate solution and then incubated at 37° C. overnight. Injections of 5 μL of each sample were made onto LC-MS (Waters Synat G2-Si) and eluted with 0.1 mL/minute of a gradient mobile phase 20-40% over 25 minutes (Mobile Phase A: 0.1% v/v FA in H₂O; Mobile Phase B: 0.1% v/v FA in Acetonitrile). The LC separation was achieved on a Waters Acquity BEH C4 column (1.0×50 mM, 1.7 μM) at 80° C.

The mass spectrometry spectra were deconvoluted using Masslynx software and the drug to antibody ratio (DAR) was calculated using the following equations:

1. Relative percentage (%) of drug (Dn) by distribution peak intensity (PI):

Dn %=PIn/Σ(PI0+PI1+PI2+PIi)×100

(n=0, 1, 2, 3, . . . , i)
2. Average DAR calculation:

DAR=Σ(1×D1%+2×D2%+3×D3%+ . . . +i×Di %)

### Example 23. Surface Plasmon Resonance Derived Binding Affinities and Kinetic Constants of Conjugated Human Monoclonal Anti-MET (Monospecific and Bispecific) Antibodies

Equilibrium dissociation constants (K_{D} values) for MET binding to anti-MET antibodies conjugated with either MCC-DM1 diastereomer (maytansinoid A) or maytansin-3-N-methyl-L-alanine-N-Me-beta-alanine-carbamyl-(p-amino)benzyl-citrulline-valine-adipoyl-succinate (Compound 1, FIG. 20) (maytansinoid B) were determined using a real-time surface plasmon resonance biosensor assay on a Biacore 2000 instrument. The Biacore sensor surface was derivatized by amine coupling with a monoclonal mouse anti-human Fc antibody (GE Healthcare, #BR-1008-39) to capture anti-MET ADC and parent unmodified antibodies expressed with human constant regions. Biacore binding studies were performed in HEPES Buffered Saline (HBS)-EP running buffer (0.01M HEPES pH 7.4, 0.15M NaCl, 3 mM EDTA, 0.05% v/v Surfactant P20). Human MET was prepared in-house expressing a C-terminal myc-myc-hexahistidine tag (hMET-mmh). Different concentrations (3-fold dilutions) of hMET-mmh (ranging from 30 nM to 1.1 nM) prepared in HBS-EP running buffer were injected over the anti-MET ADC or antibody captured surface at a flow rate of 40 μL/min. Association of hMET-mmh to each of the captured ADCs and monoclonal antibodies was monitored for 4 minutes. Subsequently, hMET-mmh dissociation was monitored for 6 minutes in HBS-EP running buffer. Anti-human Fc surface was regenerated by a brief injection of 20 mM H₃PO₄. All binding kinetic experiments were performed at 25° C.

Kinetic association (kₐ) and dissociation (k_{d}) rate constants were determined by fitting the real-time sensorgrams to a 1:1 binding model using Scrubber 2.0c curve fitting software. All sensorgrams were double referenced by subtracting buffer injection sensorgram signal from the corresponding analyte sensorgram, thereby removing artifacts caused by dissociation of the antibody from the capture surface. Binding dissociation equilibrium constants (K_{D}) and dissociative half-lives (t1/2) were calculated from the kinetic rate constants as:

$${K}_{D} ( M ) = \frac{kd}{ka} , \\ and$$

$$t ½ ( min ) = \frac{ln ( 2 )}{60 * kd}$$

Binding kinetic parameters for Maytansinoid A or Maytansinoid B conjugated anti-Met monospecific and bispecific antibodies are shown below in Table 25, with some experiments run in duplicate.

TABLE 25
Biacore Binding Affinities of Conjugated Mono- and
Bi-specific Monoclonal Anti-MET Antibodies at 25° C.

| | mAb Captured | Antigen Bound | ka | kd | K_{D} | t½ |
|---|---|---|---|---|---|---|
| Antibody | (RU) | (RU) | (1/Ms) | (1/s) | (M) | (min) |
| H4H13312P2 | 148.1 ± 1.2 | 12.3 | 2.59E+05 | 5.35E−03 | 2.07E−08 | 2.2 |
| H4H13312P2 | 142.7 ± 0.3 | 12.1 | 1.87E+05 | 4.85E−03 | 2.59E−08 | 2.4 |
| H4H13312P2- | 232.6 ± 0.5 | 11.9 | 1.82E+05 | 7.18E−03 | 3.94E−08 | 1.6 |
| Maytansinoid A | | | | | | |
| H4H13312P2- | 263.0 ± 2.6 | 10.9 | 1.80E+05 | 6.32E−03 | 3.51E−08 | 1.8 |
| Maytansinoid B | | | | | | |
| H4H14639D | 283.6 ± 4.4 | 82.8 | 5.90E+05 | 1.56E−03 | 2.64E−09 | 7.4 |
| H4H14639D- | 207.7 ± 0.8 | 55.8 | 4.95E+05 | 1.81E−03 | 3.65E−09 | 6.4 |
| Maytansinoid A | | | | | | |
| H4H14639D- | 227.5 ± 0.4 | 55.4 | 4.83E+05 | 1.87E−03 | 3.86E−09 | 6.2 |
| Maytansinoid B | | | | | | |
| H4H14639D- | 284.0 ± 1.1 | 62.8 | 4.70E+05 | 1.76E−03 | 3.74E−09 | 6.6 |
| Maytansinoid A | | | | | | |
| H4H14639D- | 268.7 ± 0.7 | 72.8 | 4.91E+05 | 1.45E−03 | 2.95E−09 | 8.0 |
| Maytansinoid B | | | | | | |

### Example 24: In Vitro Potencies of Anti-MET Antibody Drug Conjugates (ADCs)

To determine the relative cell-killing potency of anti-MET antibody drug conjugates (ADCs) described herein, cell-killing assays were run on multiple cells lines expressing varying levels of endogenous MET. EBC-1 (Riken Cell Bank; #RBRC-RCB1965), MKN-45 (JCRB; #JCRB0254), NCI-H1993 (ATCC; #CRL-5909), and J.RT3 (ATCC; #TIB-153) cell lines were maintained in RPMI+10% FBS+1× penicillin/streptomycin/L-glutamine (P/S/G), SNU-5 (ATCC; #CRL-5973) were maintained in Iscove's+10% FBS+1× P/S/G, Hs746t (ATCC; #HTB-135) and HEK293 (ATCC; #003041) were maintained in DME+10% FBS+1× P/S/G, MDA-MB-231 (ATCC; #HTB-26) were maintained in Liebowitz's L-15+10% FBS+1× P/S/G+1× nonessential amino acids (NEAA) without CO₂, U87MG (ATCC; #HTB-14) were maintained in MEM Earle's Salts+15% FBS+1× P/S/G+1×NEAA, T47D (ATCC; #HTB-133) were maintained in RPM1 1640+10% FBS+1× P/S/G+10 mM HEPES+1 mM sodium pyruvate+10 ug/ml Bovine Insulin, and A549 (ATCC; #CCL-185) were maintained in Kaighn's Nutrient Mixture F-12 (HAM's F-12K)+10% FBS+1× P/S/G.

Initially, relative binding of the anti-MET antibodies was assessed with unconjugated H4H14635D, H4H14639D and H4H13312P2 antibodies across the entire panel of cell lines via flow cytometry. Briefly, 1×10⁶ cells were incubated with 10 μg/ml of H4H14635D, H4H14639D, H4H13312P2 or an isotype control antibody (REGN1945) for 30 minutes on ice in PBS+2% FBS (FACS buffer). Following one wash with FACS buffer, cells were incubated with 10 μg/ml of Alexa647 conjugated anti-human secondary antibody (Jackson ImmunoResearch, #109-606-170) for 30 minutes on ice. After one additional wash with FACS buffer, samples were fixed with Cytofix (BD Biosciences, #554655), filtered with FACS buffer and run on an iQue flow cytometer (Intelicyte). Mean fluorescence intensity (MFI) data was determined using FlowJo software (FlowJo LLC). FACS binding is expressed as fold MFI binding above isotype control levels, and results are summarized in Table 26. Relative binding of the three anti-Met antibodies was comparable on each cell line and ranged from 447-fold to 7-fold above isotype controls. No detectable binding of any of the 3 anti-MET antibodies tested was observed on T47D, HEK293, or J.RT3 cells.

To measure in vitro cytotoxicity of anti-MET ADCs, nuclear counts after a 3 or 6-day treatment with the ADCs was assessed. Briefly, cells were seeded in 96 well collagen coated plates (Greiner, VWR; #82050-812) at 750-3000 cells/well in complete growth media and grown overnight at 37° C., 5%002. For cell viability curves, serially diluted ADCs, unconjugated antibodies, or free payloads were added to the cells at final concentrations ranging from 100 nM to 0.01 nM (based on toxin concentration) and incubated for 3 or 6 days at 37° C. in 5% CO₂. Cells were subsequently treated with 3 ug/ml Hoechst 33342 nuclear stain (Invitrogen, #H3570) while being fixed with 4% formaldehyde. Images were acquired on the ImageXpress micro XL (Molecular Devices, Sunnyvale, Calif.) and nuclear counts were determined via MetaXpress image analysis software (Molecular Devices, Sunnyvale, Calif.). Background nuclear counts from cells treated with 40 nM digitonin were subtracted from all wells and viability was expressed as a percentage of the untreated controls. IC₅₀ values were determined from a four-parameter logistic equation over a 10-point response curve (Graph Pad Prism). The untreated condition for each dose-response curve is also included in the analysis and is represented as the lowest dose. IC₅₀ values and percent cell killing are shown in Tables 27 and 28.

As summarized in Table 27, the anti-MET antibody-drug conjugate H4H14639D-Maytansinoid A specifically reduced cell viability in Met amplified EBC-1, SNU-5, MKN-45, NCI-H1993, and Hs746t cell backgrounds with IC₅₀ values ranging from 0.35 nM to 0.96 nM. The percentage of cells killed (max % kill) ranged from 73% to 100%. H4H14639D-Maytansinoid A also specifically killed 84% of A549 cells with an IC₅₀ values of 13.91 nM. H4H14639D-Maytansinoid A IC₅₀ values were greater than 37 nM in low expressing (MDA-MB-231 and U87MG) and non-expressing (T47D, HEK293, and J.RT3) cell lines. The similarly conjugated isotype control antibody killed all cell lines with IC₅₀ values greater than 35 nM. The methyl disulfide version of DM1 (MeS-DM1) killed all tested lines with IC₅₀ values ranging from 0.07 nM to 2.86 nM.

In a separate experiment, three anti-Met antibodies (H4H14639D, H4H14635D, and H4H13312P2) were conjugated to Maytansinoid A or Maytansinoid B maytansinoid payloads, and in vitro cytotoxicity was assessed in EBC-1, Hs746t, A549, ant T47D cells following a 6 day treatment. As summarized in Table 28, all anti-Met antibody-drug conjugates potently and specifically reduced cell viability in Met positive cells, with IC₅₀ values as low as 10 μM in EBC-1 cells, 0.82 nM in Hs746t cells, and 3.5 nM in A549 cells. The percentage of cells killed was greater than 95% in EBC-1 cells, greater than 86% in Hs746t cells, and greater than 72% in A549 cells. T47D cells (Met negative) were not specifically killed by the anti-Met ADCs. The similarly conjugated isotype control antibodies reduced cell viability in all of the tested cell lines with IC₅₀ values greater than 5 nM in EBC-1 cells, greater than 33 nM in Hs746t cells, and greater than 90 nM in A549 and T47D cells. Unconjugated H4H14639D reduced cell viability in EBC-1, Hs746t, and A549 cells but at a lower percentage than the conjugated antibodies. Unconjugated H4H14635D and H4H13312P2 had little to no impact on viability in any of the tested cell lines. The methyl disulfide version of DM1 (MeS-DM1) killed all tested lines with IC₅₀ values ranging from 0.12 nM to 1.39 nM. In contrast, M24 (the payload released from Maytansinoid B) killed cells with IC50s >100 nM.

TABLE 26
FACS Binding of Unconjugated MET Antibodies to Tumor Cell Lines.

| | FACS Binding (MFI Fold Above Isotype Control) | | | | | |
|---|---|---|---|---|---|---|
| | | Secondary | REGN1945 | | | |
| Cell Line | Unstained | Alone | (Isotype Control) | H4H14635D | H4H14639D | H4H13312P2 |
| EBC-1 | 0.7 | 0.6 | 1 | 263 | 252 | 147 |
| SNU-5 | 1 | 1.2 | 1 | 477 | 454 | 235 |
| MKN-45 | 1 | 0.8 | 1 | 183 | 156 | 94 |
| NCI-H1993* | 1 | 2 | ND | ND | 188 | 188 |
| Hs746t | 0.8 | 1.1 | 1 | 39 | 34 | 27 |
| MDA-MB-231 | 3 | 5.6 | 1 | 11 | 12 | 7 |
| U87MG | 1.6 | 1.7 | 1 | 18 | 18 | 10 |
| T47D | 1 | 0.9 | 1 | 1.3 | 1 | 1.4 |
| A549 | 0.7 | 0.5 | 1 | 12 | 10 | 7 |
| HEK293 | 0.2 | 0.2 | 1 | 1.8 | 1.8 | 1.2 |
| J.RT3 | 0.8 | 1 | 1 | 1.6 | 1.4 | 1.1 |

*Expressed as fold above unstained for NCI-H1993.

TABLE 27
IC₅₀ and Max % Kill of Anti-MET ADCs in 3-Day in vitro Cytotoxicity Assay.

| | | | |
|---|---|---|---|
| EBC-1 | SNU-5 | MKN-45 | NCI-H1993 |

| Antibody-Drug | IC₅₀ | Max % | IC₅₀ | Max % | IC₅₀ | Max % | IC₅₀ | Max % |
|---|---|---|---|---|---|---|---|---|
| Conjugate | (nM) | Kill | (nM) | Kill | (nM) | Kill | (nM) | Kill |
| DM1 (MeS-DM1) | 2.22 | 90 | 1.22 | 99 | 2.73 | 85 | 2.86 | 81 |
| H4H14639D | 0.82 | 37 | 0.30 | 40 | ND | 0 | ND | 0 |
| H4H14639D- | 0.96 | 89 | 0.40 | 100 | 0.35 | 86 | 0.41 | 94 |
| Maytansinoid A | | | | | | | | |
| REGN1945- | 35.06 | 65 | >100 | 14 | >100 | 39 | 49.42 | 68 |
| Maytansinoid A | | | | | | | | |

| | Hs746t | | MDA-MB-231 | | U87MG | |
|---|---|---|---|---|---|---|
| Antibody-Drug | IC₅₀ | Max % | IC₅₀ | Max % | IC₅₀ | Max % |
| Conjugate | (nM) | Kill | (nM) | Kill | (nM) | Kill |
| DM1 (free drug) | 1.46 | 81 | 1.53 | 89 | 0.61 | 89 |
| H4H14639D | 0.42 | 7 | >100 | 6 | >100 | 6 |
| H4H14639D- | 0.56 | 73 | >100 | 48 | 100 | 58 |
| Maytansinoid A | | | | | | |
| REGN1945- | 33.22 | 44 | >100 | 42 | 94.71 | 58 |
| Maytansinoid A | | | | | | |

| | T47D | | A549 | | HEK293 | | J.RT3 | |
|---|---|---|---|---|---|---|---|---|
| Antibody-Drug | IC₅₀ | Max % | IC₅₀ | Max % | IC₅₀ | Max % | IC₅₀ | Max % |
| Conjugate | (nM) | Kill | (nM) | Kill | (nM) | Kill | (nM) | Kill |
| DM1 (free drug) | 1.33 | 91 | 2.56 | 97 | 0.15 | 95 | 0.07 | 100 |
| H4H14639D | >100 | 0 | >100 | 37 | ND | 0 | >100 | 5 |
| H4H14639D- | >100 | 6 | 13.91 | 84 | 40.90 | 65 | 37.82 | 59 |
| Maytansinoid A | | | | | | | | |
| REGN1945- | >100 | 1 | >100 | 63 | >100 | 44 | 39.79 | 70 |
| Maytansinoid A | | | | | | | | |

TABLE 28
IC₅₀ and Max % Kill of Anti-MET ADCs in 6-day in vitro Cytotoxicity Assay.

| | EBC-1 | | Hs746t | | T47D | | A549 | |
|---|---|---|---|---|---|---|---|---|
| Antibody-Drug | IC₅₀ | Max % | IC₅₀ | Max % | IC₅₀ | Max % | IC₅₀ | Max % |
| Conjugate | (nM) | Kill | (nM) | Kill | (nM) | Kill | (nM) | Kill |
| DM1 (MeS-DM1) | 0.12 | 62 | 1.39 | 88 | 0.24 | 96 | 0.49 | 90 |
| M24 (Maytansinoid B | >100 | 32 | >100 | 10 | >100 | 0 | >100 | 10 |
| released payload) | | | | | | | | |
| H4H14639D | 0.37 | 66 | 0.44 | 35 | >100 | 0 | 0.17 | 29 |
| H4H14639D- | 0.27 | 97 | 0.82 | 87 | >100 | 3 | 6.01 | 86 |
| Maytansinoid A | | | | | | | | |
| H4H14639D- | 0.01 | 96 | 0.86 | 90 | >100 | 0 | 3.54 | 80 |
| Maytansinoid B | | | | | | | | |
| H4H13312P2 | >100 | 30 | >100 | 0 | >100 | 0 | >100 | 7 |
| H4H13312P2- | 0.39 | 95 | 1.59 | 87 | >100 | 6 | 18.30 | 89 |
| Maytansinoid A | | | | | | | | |
| H4H13312P2- | 0.07 | 95 | 0.89 | 90 | >100 | 3 | 27.10 | 85 |
| Maytansinoid B | | | | | | | | |
| H4H14635D | >100 | 11 | >100 | 7 | >100 | 7 | >100 | 0 |
| H4H14635D- | 0.76 | 96 | 1.76 | 86 | >100 | 92 | 6.78 | 91 |
| Maytansinoid A | | | | | | | | |
| H4H14635D- | 0.26 | 96 | 2.32 | 89 | >100 | 2 | 21.40 | 72 |
| Maytansinoid B | | | | | | | | |
| REGN1945 | >100 | 0 | >100 | 0 | >100 | 0 | >100 | 1 |
| REGN1945- | 28.08 | 93 | 33.06 | 76 | >100 | 14 | 93.40 | 49 |
| Maytansinoid A | | | | | | | | |
| REGN1945- | 5.01 | 97 | >100 | 0 | >100 | 1 | >100 | 15 |
| Maytansinoid B | | | | | | | | |

### Example 25: In Vivo Efficacy Against Gastric Cancer Cells

3 million Hs746T gastric cancer cells were implanted subcutaneously into the flank of C.B.-17 SCID mice. Once the tumor volumes reached approximately 150 mm³, mice were randomized into groups of 6 and were treated with control antibodies REGN1945-Maytansinoid B or REGN1945-Maytansinoid A at 10 mg/kg or with H4H14639D-Maytansinoid A or H4H14639D-Maytansinoid B at 3 or 10 mg/kg. All antibodies were administered three times at a frequency of once per week. Tumor growth was monitored for 37 days post-implantation.

The effect of H4H14639D-Maytansinoid A or H4H14639D-Maytansinoid B on the growth of human tumor xenografts in immunocompromised mice was assessed, and the results are shown in Table 29. Tumors treated with the control antibodies, REGN1945-Maytansinoid B or REGN1945-Maytansinoid A, grew to reach protocol size limits within 20 days. Tumors treated with H4H14639D-Maytansinoid A at 3 mg/kg grew to reach protocol size limits within 27 days. Growth of tumors treated with H4H14639D-Maytansinoid B at 3 mg/kg was inhibited for the duration of the experiment. Treatment of tumors with H4H14639D-Maytansinoid A or H4H14639D-Maytansinoid B at 10 mg/kg induced regression of tumor size relative to the beginning of treatment.

TABLE 29
Tumor Growth in SCID Mice Treated
with Anti-Met-C Antibody Conjugates

| | Tumor growth (mm³) |
|---|---|
| | from start of treatment |
| Antibody (mg/kg) | (mean ± SD) |
| REGN1945-Maytansinoid A 10 mg/kg | 1244 ± 199 |
| REGN1945-Maytansinoid B 10 mg/kg | 1345 ± 121 |
| H4H14639D-Maytansinoid A 3 mg/kg | 832 ± 15 |
| H4H14639D-Maytansinoid A 10 mg/kg | −148 ± 0.17 |
| H4H14639D-Maytansinoid B 3 mg/kg | 19 ± 147 |
| H4H14639D -Maytansinoid B 10 mg/kg | −137 ± 0 |

### Example 26: In Vivo Efficacy Against Lung Cancer Cells

5 million EBC1 lung cancer cells were implanted subcutaneously into the flank of C.B.-17 SCID mice. Once the tumor volumes reached approximately 170 mm³, mice were randomized into groups of 6 and were treated with control antibody REGN1945-Maytansinoid B at 15 mg/kg or H4H14639D-Maytansinoid B at 2.5, 5, 10 or 15 mg/kg. Antibodies were administered two times at a frequency of once per week. Tumor growth was monitored for 73 days post-implantation.

The effect of H4H14639D on the growth of human tumor xenografts in immunocompromised mice was assessed. Tumors treated with the control antibody, REGN1945-Maytansinoid B, grew to reach protocol size limits within 24 days (IACUC protocols require sacrifice of animals harboring tumors that exceed 2 cm in diameter, approximately 1500 mm³). Treatment of tumors with H4H14639D-Maytansinoid B at 2.5, 5, 10 or 15 mg/kg induced regression of tumor size relative to the beginning of treatment. Results are shown in Table 30.

TABLE 30
Tumor Growth in SCID Mice Treated
with Anti-Met-C Antibody Conjugates

| | Tumor growth (mm³) |
|---|---|
| | from start of treatment |
| Antibody (mg/kg) | (mean ± SD) |
| REGN1945-Maytansinoid B 15 mg/kg | 1106 ± 165 |
| H4H14639D-Maytansinoid B 2.5 mg/kg | −142 ± 24 |
| H4H14639D-Maytansinoid B 5 mg/kg | −163 ± 0 |
| H4H14639D-Maytansinoid B 10 mg/kg | −173 ± 0 |
| H4H14639D-Maytansinoid B 15 mg/kg | −179 ± 0 |

### Example 27: In Vivo Efficacy Against Patient-Derived NSCLC Tumors

Met-expressing NSCLC CTG-0165 patient-derived tumors were implanted subcutaneously into the flank of nu/nu Nude mice. Once the tumor volumes reached approximately 150 mm³, mice were randomized into groups of 6 and were treated with control antibodies REGN1945-Maytansinoid B or REGN1945-Maytansinoid A at 10 mg/kg or with H4H14639D-Maytansinoid A or H4H14639D-Maytansinoid B at 3 or 10 mg/kg. All antibodies were administered three times at a frequency of once per week. Tumor growth was monitored for 61 days post-implantation.

The effect of H4H14639D-Maytansinoid A or H4H14639D-Maytansinoid B on the growth of human tumor xenografts in immunocompromised mice was assessed. Tumors treated with the control antibodies REGN1945-Maytansinoid A or REGN1945-Maytansinoid B grew to reach protocol size limits within 27 days. Growth of tumors treated with H4H14639D-Maytansinoid A or H4H14639D-Maytansinoid B at 3 mg/kg was inhibited for 27 days. Treatment of tumors with H4H14639D-Maytansinoid A or H4H14639D-Maytansinoid B at 10 mg/kg induced regression of tumor size relative to the beginning of treatment. Data are provided in Table 31.

TABLE 31
Tumor Growth in Nude Mice Treated
with Anti-Met-C Antibody Conjugates

| | Tumor growth (mm³) |
|---|---|
| | from start of treatment |
| Antibody (mg/kg) | (mean ± SD) |
| REGN1945-Maytansinoid A 10 mg/kg | 967 ± 136 |
| REGN1945-Maytansinoid B 10 mg/kg | 1537 ± 373 |
| H4H14639D-Maytansinoid A 3 mg/kg | 154 ± 227 |
| H4H14639D-Maytansinoid A 10 mg/kg | “−141 ± 2.3 |
| H4H14639D-Maytansinoid B 3 mg/kg | 517 ± 362 |
| H4H14639D-Maytansinoid B 10 mg/kg | “−145 ± 2 |

### Example 28: Hydrogen/Deuterium (H/D) Exchange Based Epitope Mapping Epitope Mapping of Anti-Met Antibodies H4H13312P2, H4H13306P2 and H4H14639D Binding to Human MET

Experiments were conducted to determine the specific regions of human hepatocyte growth factor receptor ectodomain (SEQ ID NO:155: human Met isoform 1 (Uniprot ID: P08581) expressed with a myc-myc-hexahistidine(.mmh) tag; hereafter referred to as hMet) with which anti-Met antibodies H4H13312P2, H4H13306P2 and H4H14639D interact. H4H13312P2 and H4H13306P2 are bivalent-monospecific anti-Met antibodies; H4H14639D is a bispecific antibody comprising two heavy chains binding to distinct epitopes on Met, each from H4H13312P2 and H4H13306P2, respectively, and a universal light chain. (See Example 5).

Hydrogen/Deuterium (H/D) Exchange epitope mapping with mass spectrometry (HDX-MS) was utilized to determine the binding epitopes of the antibodies mentioned above. A general description of the HDX method is set forth in e.g., Ehring (1999) Analytical Biochemistry 267(2):252-259; and Engen and Smith (2001) Anal. Chem. 73:256A-265A.

### Experimental Procedure

To map the binding epitope(s) of anti-Met antibodies H4H13312P2, H4H13306P2 and H4H14639D on hMET via HDX, the individual antibodies were separately covalently attached to NHS-activated Sepharose 4 Fast Flow beads (GE Healthcare, Pittsburgh, Pa.). Two methods “On-Antigen” and “On-Complex”, as described below, were utilized to confirm the binding epitopes of the anti-Met antibodies.

In the ‘On-Antigen’ experimental condition, hMET was deuterated for 5.0 mins or 10.0 mins in PBS buffer prepared with D20. The deuterated antigen was bound to H4H13312P2 or H4H13306P2 antibody beads through a short incubation, and then eluted from beads with an ice-cold low pH quench buffer. The eluted sample was manually loaded to a Waters H/DX-MS system consisting of integrated online peptide digestion, trapping, 9.0 minute Liquid Chromatography (LC) separation, and Synapt G2-Si MS data acquisition.

In the ‘On-Complex’ experimental condition, hMET was first bound to H4H13312P2 or H4H13306P2 beads and then deuterated for 5.0 mins or 10.0 mins via incubation in PBS buffer prepared with D20. The deuterated hMET was eluted and analyzed by the Waters H/DX-MS system as mentioned above.

For the identification of the peptic peptides from hMET, LC-MS^{E} data from the un-deuterated sample were processed and searched against human MET using Waters ProteinLynx Global Server (PLGS) software. The identified peptides were imported to DynamX 3.0 software and filtered by the following two criteria: 1) minimum products per amino acid is 0.3; 2) replication file threshold is 3.0. DynamX 3.0 software subsequently automatically calculated the deuterium uptake difference of each identified peptide between ‘On-Antigen’ and ‘On-Complex” across both 5 min and 10 min deuteration time points. The individual isotopic peak of each peptide picked up by DynamX software for the centroid value calculation was also manually examined to ensure the accuracy of the deuterium uptake calculation.

In general, delta values for deuteration above 0.2 were used as the cut-off point for determining a specific binding epitope.

### Results

Using online pepsin digestion via Waters Enzymate™ BEH Pepsin Column (2.1×30 mm, 5 μm) coupled with 9.0 minute LC-MS^{E} data acquisition, a total of 162 peptic peptides from human MET were reproducibly identified with traceable deuterium uptake for both ‘On-Antigen’ and ‘On-Complex’ experiments when the H4H13312P2 antibody beads were used. These peptides represent 55.7% sequence coverage. Among all these peptides, only five were found to have significantly reduced deuteration uptake upon binding H4H13312P2 (‘On-Complex’) as compared to the deuteration of the antigen alone (‘On-Antigen’). The centroid values of these five peptides under both the experimental conditions were illustrated in Table 32. The region corresponding to the residues 192-204 covered by these five peptides were defined as the binding epitope for the antibody H4H13312P2 based on HDX data.

TABLE 32
hMET peptic peptides with reduced deuterium
uptake upon binding to H4H13312P2

| | 5 min Deuteration | | | 10 min Deuteration | | |
|---|---|---|---|---|---|---|
| | On- | On- | | On- | On- | |
| | Complex | Antigen | | Complex | Antigen | |
| Residues | Centroid | Centroid | | Centroid | Centroid | |
| of hMET | MH+ | MH+ | Δ | MH+ | MH+ | Δ |
| 192-202 | 1351.25 | 1351.83 | −0.58 | 1351.39 | 1352.27 | −0.88 |
| 192-203 | 1482.34 | 1482.94 | −0.60 | 1482.50 | 1483.40 | −0.90 |
| 192-204 | 1629.84 | 1630.71 | −0.87 | 1630.01 | 1631.10 | −1.09 |
| 193-202 | 1252.07 | 1252.79 | −0.72 | 1252.25 | 1253.08 | −0.83 |
| 193-203 | 1383.22 | 1383.79 | −0.57 | 1383.40 | 1384.17 | −0.77 |

For the HDX experiment carried out using H4H13306P2 antibody beads, a total of 98 peptic peptides from hMET were reproducibly identified with traceable deuterium uptake during both ‘On-Antigen’ and ‘On-Complex’ experiments. These 98 peptides represent 52.1% sequence coverage. Among all these peptides, twelve were observed to have reduced have significantly reduced deuteration uptake upon binding H4H13306P2 (‘On-Complex’) as compared to the deuteration of the antigen alone (‘On-Antigen’). The centroid values of these twelve peptides under both the experimental conditions were illustrated in Table 33. The regions corresponding to residues 305-315 and residues 421-455 covered by these peptides were defined as the binding epitope for the antibody H4H13306P2 based on HDX data.

TABLE 33
hMET peptic peptides with reduced deuterium
uptake upon binding to H4H13306P2

| | 5 min Deuteration | | | 10 min Deuteration | | |
|---|---|---|---|---|---|---|
| | On- | On- | | On- | On- | |
| | Complex | Antigen | | Complex | Antigen | |
| Residues | Centroid | Centroid | | Centroid | Centroid | |
| of hMET | MH+ | MH+ | Δ | MH+ | MH+ | Δ |
| 305-312 | 818.20 | 818.83 | −0.63 | 818.31 | 819.13 | −0.82 |
| 305-315 | 1161.50 | 1162.58 | −1.08 | 1161.80 | 1162.95 | −1.15 |
| 306-313 | 818.48 | 818.97 | −0.49 | 818.71 | 819.28 | −0.57 |
| 421-431 | 1206.24 | 1206.75 | −0.51 | 1206.28 | 1206.95 | −0.67 |
| 421-435 | 1581.28 | 1581.84 | −0.56 | 1581.41 | 1582.09 | −0.68 |
| 421-438 | 1941.58 | 1942.15 | −0.57 | 1941.71 | 1942.39 | −0.68 |
| 422-438 | 1794.58 | 1795.04 | −0.46 | 1794.72 | 1795.34 | −0.62 |
| 439-447 | 963.90 | 964.83 | −0.93 | 963.97 | 965.24 | −1.27 |
| 439-455 | 1846.58 | 1847.79 | −1.21 | 1847.24 | 1847.85 | −0.61 |
| 439-456 | 1960.24 | 1961.32 | −1.08 | 1960.83 | 1961.42 | −0.59 |
| 441-455 | 1586.30 | 1587.71 | −1.41 | 1587.33 | 1587.79 | −0.46 |
| 442-455 | 1487.50 | 1488.50 | −1.00 | 1487.92 | 1488.54 | −0.62 |

The same methodology as outlined above was used to determine the binding epitopes for bispecific anti-Met antibody H4H14639D. The H4H14639D binding epitopes on hMET, determined by this methodology, correspond to the epitopes determined for the parental antibodies.

Binding epitope of Anti-Met antibody H4H13312P2: AA 192-204: VRRLKETKDGFMF (SEQ ID NO: 156) of SEQ ID NO: 155.

Binding epitope of Anti-Met antibody H4H13306P2: AA 305-315: LARQIGASLND (SEQ ID NO: 157) of SEQ ID NO: 155 and AA 421-455: FIKGDLTIANLGTSEGRFMQVVVSRSGPSTPHVNF (SEQ ID NO: 158) of SEQ ID NO: 155.

### Example 29: Inhibition of Cell Proliferation and Cell Viability by MET×MET Bispecific Antibody ADC in Uveal Melanoma Cell Lines

The bispecific c-Met antibody H4H14639D conjugated to one of two maytansinoid payloads and designated H4H14639D-Maytansinoid A and H4H14639D-Maytansinoid B was tested in uveal melanoma cell lines to determine effects on cell proliferation and cell viability relative to c-Met expression in the cell lines.

In a first experiment, uveal melanoma cells that express c-Met, OMM1.3, Mel202, Mel270 and MP65, were seeded overnight in 96-well plates at 1,000 cells per well in RPMI with 10% FBS and incubated at 37° C. with 5% CO₂. The cells were treated for seven days with increasing doses of REGN1945, REGN1945-Maytansinoid A, REGN1945-Maytansinoid B, H4H14639D, H4H14639D-MAYTANSINOID A and H4H14639D-Maytansinoid B from 0.01 nM up to 100 nM. After 7 days, relative cell viability was determined by measuring the reduction of WST-8 in the colorimetric assay, Dojindo Cell Counting Kit 8, using Emax Plus Microplate Reader (Molecular Devices).

In a second experiment, uveal melanoma cells that express c-Met, OMM1.3, as well as c-Met negative OCM3 cells, were seeded overnight in 96-well plates at 1,000 cells per well in RPMI with 10% FBS and incubated at 37° C. with 5% CO₂. The cells were treated with increasing doses of REGN1945, REGN1945-Maytansinoid B, H4H14639D and H4H14639D-Maytansinoid B from 0.3125 nM up to 10 nM. After 7 days, relative cell viability was determined by measuring the reduction of WST-8 in the colorimetric assay, Dojindo Cell Counting Kit 8, using Emax Plus Microplate Reader (Molecular Devices).

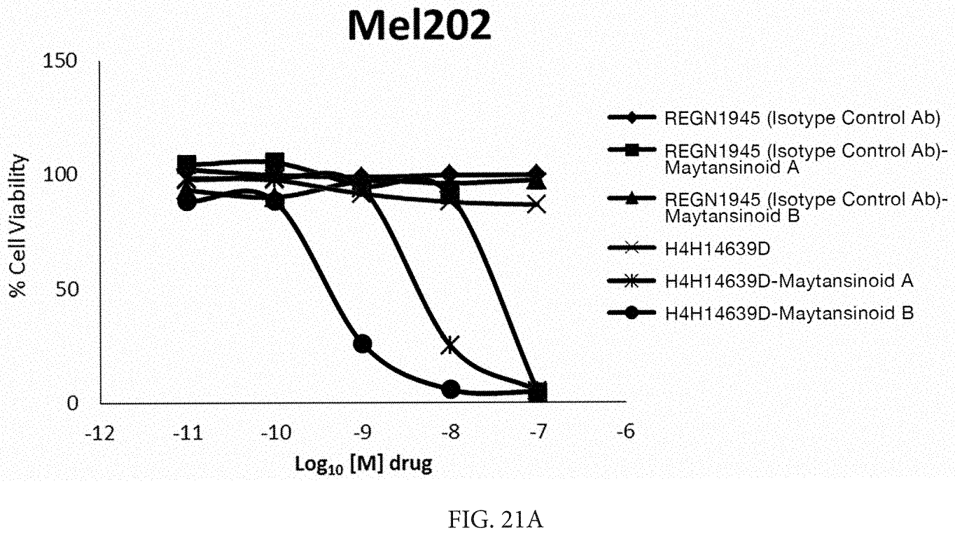

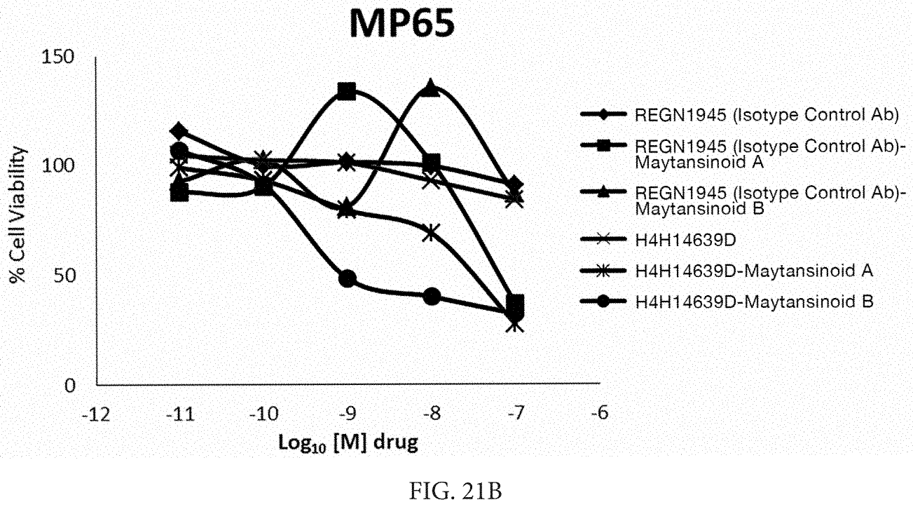

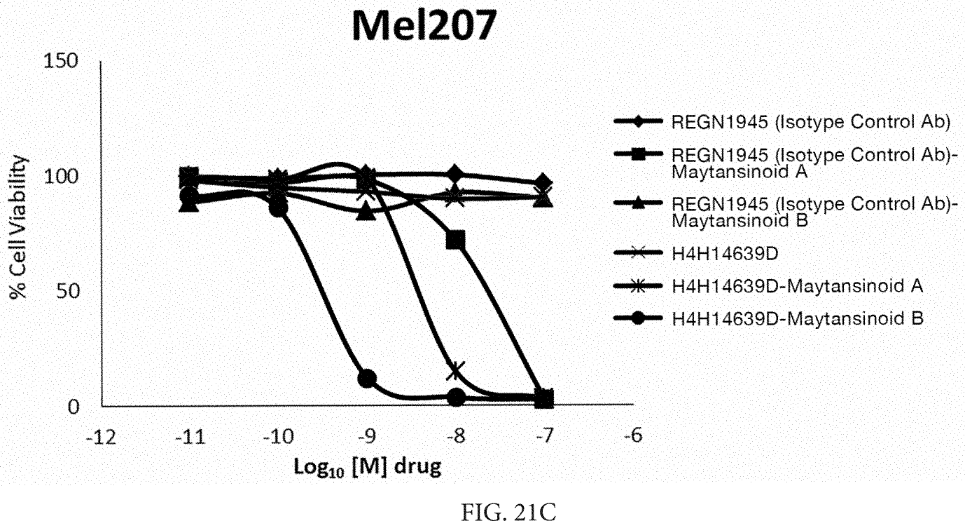

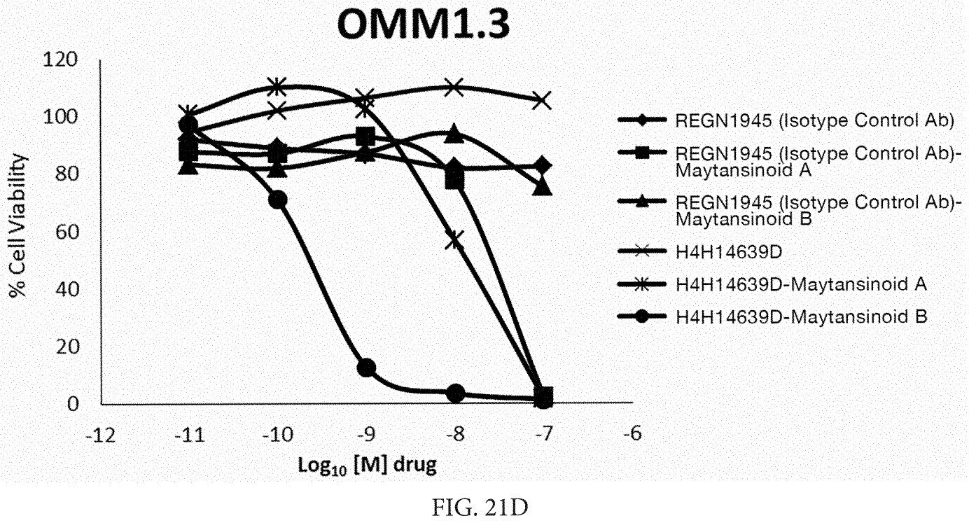

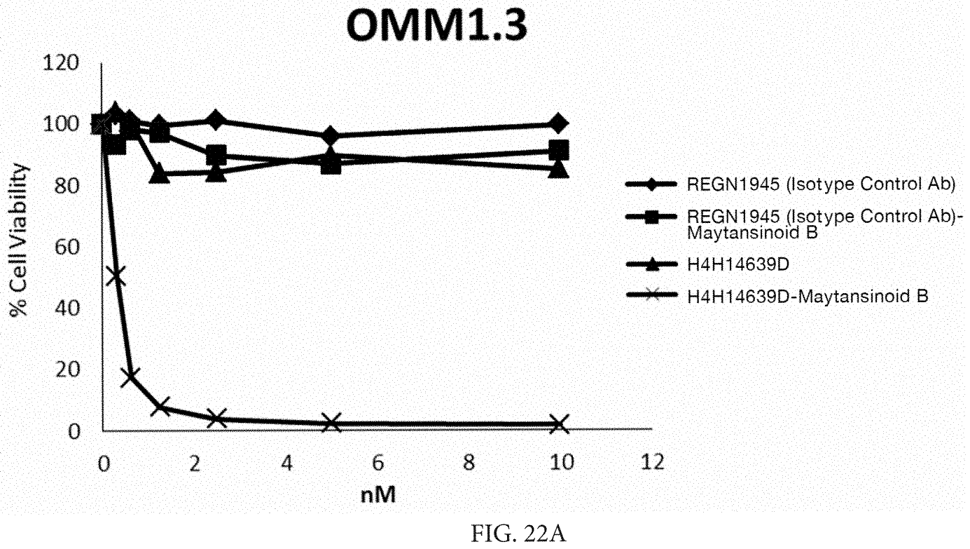

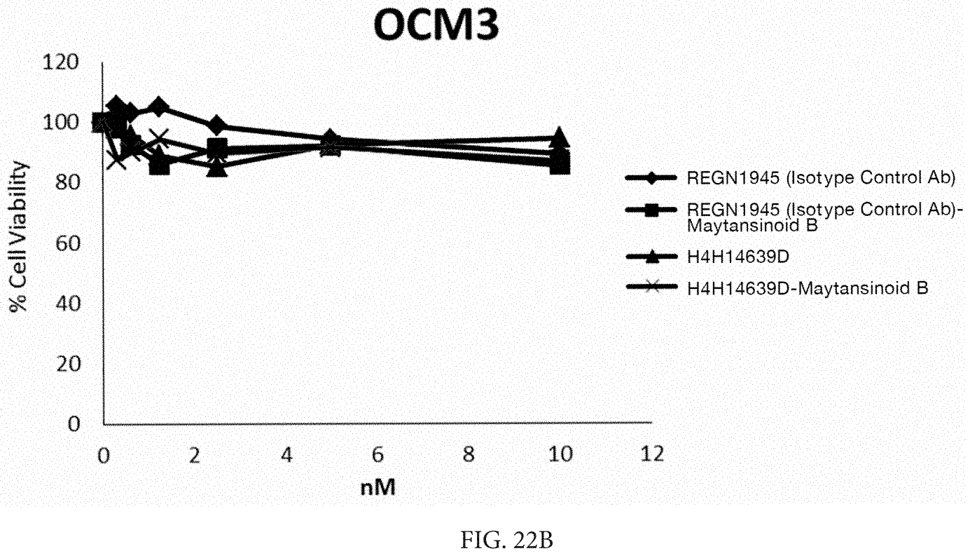

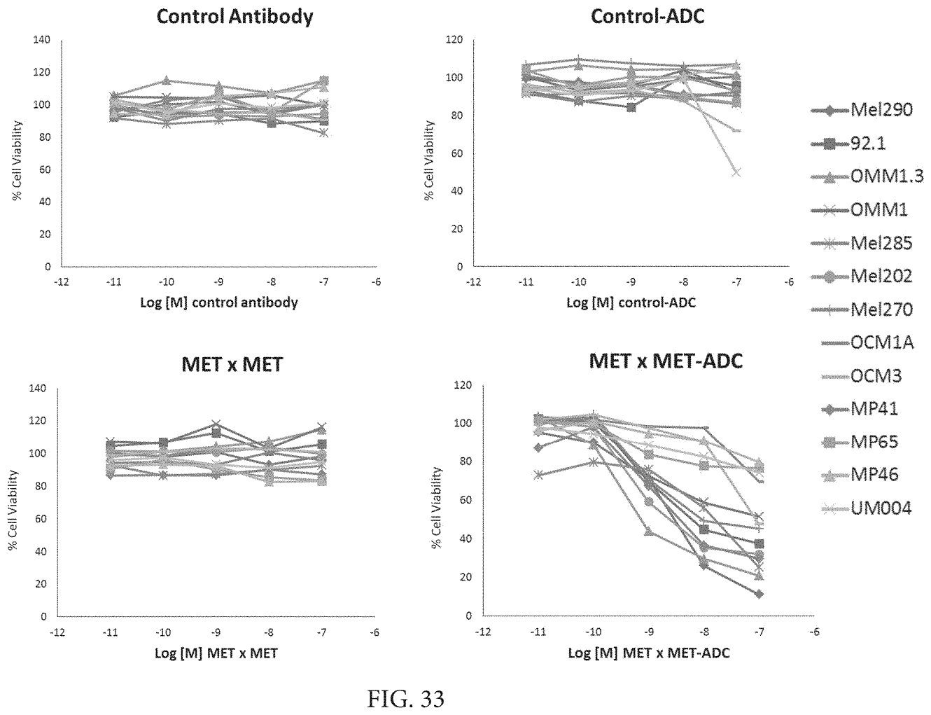

Tables 34-38 and FIGS. 21 and 22 show that the bispecific c-Met antibody conjugated to a maytansinoid payload, H4H14639D-Maytansinoid B, decreases the viability of uveal melanoma cells that express the c-Met protein relative to the control treatments. H4H14639D-Maytansinoid B had no effect on the viability of the c-Met negative cell line. FIG. 21, in log scale, depicts the impact on viability of the cells at lower ADC concentrations. H4H14639D-Maytansinoid A data are also shown in FIG. 21. The unconjugated antibody H4H14639D did not significantly reduce the viability of c-Met expressing uveal melanoma cells, indicating that these cells are not dependent on Met signaling for survival. Data from a third experiment in which thirteen cell lines were treated with increasing doses of REGN1945, REGN1945-Maytansinoid B, H4H14639D and H4H14639D-Maytansinoid B over 3 days is shown in FIG. 33. H4H14639D-Maytansinoid B decreases the viability of MET expressing uveal melanoma cell lines in a dose-dependent manner with an IC50 of less than 1 nM.

TABLE 34
% Viability of Mel270 cells after
H4H14639D-Maytansinoid B treatment

| Mel270 | % Cell Viability (n = 3) |
|---|---|
| REGN1945 1 nM | 100.13 ± 3.46 |
| REGN1945 10 nM | 98.20 ± 4.38 |
| REGN1945-Maytansinoid B 1 nM | 84.35 ± 10.79 |
| REGN1945-Maytansinoid B 10 nM | 92.26 ± 4.86 |
| H4H14639D 1 nM | 92.77 ± 4.49 |
| H4H14639D 10 nM | 89.61 ± 5.06 |
| H4H14639D-Maytansinoid B 1 nM | 11.96 ± 0.51 |
| H4H14639D-Maytansinoid B 10 nM | 3.59 ± 0.33 |

TABLE 35
% Viability of Mel202 cells after
H4H14639D-Maytansinoid B treatment

| Mel202 | % Cell Viability (n = 3) |
|---|---|
| REGN1945 1 nM | 98.80 ± 99.46 |
| REGN1945 10 nM | 90.74 ± 9.03 |
| REGN1945-Maytansinoid B 1 nM | 96.86 ± 5.29 |
| REGN1945-Maytansinoid B 10 nM | 95.90 ± 8.12 |
| H4H14639D 1 nM | 91.36 ± 10.57 |
| H4H14639D 10 nM | 87.74 ± 5.43 |
| H4H14639D-Maytansinoid B 1 nM | 25.82 ± 0.36 |
| H4H14639D-Maytansinoid B 10 nM | 5.80 ± 0.21 |

TABLE 36
% Viability of OMM1.3 cells after
H4H14639D-Maytansinoid B treatment

| OMM1.3 | % Cell Viability (n = 3) |
|---|---|
| REGN1945 1 nM | 86.86 ± 4.46 |
| REGN1945 10 nM | 81.89 ± 5.13 |
| REGN1945-Maytansinoid B 1 nM | 87.37 ± 12.49 |
| REGN1945-Maytansinoid B 10 nM | 93.66 ± 11.17 |
| H4H14639D 1 nM | 106.30 ± 4.76 |
| H4H14639D 10 nM | 109.87 ± 20.36 |
| H4H14639D-Maytansinoid B 1 nM | 12.60 ± 0.60 |
| H4H14639D-Maytansinoid B 10 nM | 3.66 ± 0.65 |

TABLE 37
% Viability of MP65 cells after H4H14639D-
Maytansinoid B treatment

| MP65 | % Cell Viability (n = 3) |
|---|---|
| REGN1945 1 nM | 101.40 ± 33.52 |
| REGN1945 10 nM | 99.58 ± 11.88 |
| REGN1945-Maytansinoid B 1 nM | 81.21 ± 27.03 |
| REGN1945-Maytansinoid B 10 nM | 135.27 ± 54.14 |
| H4H14639D 1 nM | 101.10 ± 28.58 |
| H4H14639D 10 nM | 92.87 ± 40.98 |
| H4H14639D-Maytansinoid B 1 nM | 48.43 ± 14.45 |
| H4H14639D-Maytansinoid B 10 nM | 40.00 ± 7.10 |

TABLE 38
% Viability of OCM3 cells after H4H14639D-
Maytansinoid B treatment

| OCM3 | % Cell Viability (n = 3) |
|---|---|
| REGN1945 1.25 nM | 104.25 ± 6.73 |
| REGN1945 10 nM | 89.64 ± 7.83 |
| REGN1945-Maytansinoid B 1.25 nM | 88.16 ± 15.49 |
| REGN1945-Maytansinoid B 10 nM | 87.56 ± 15.08 |
| H4H14639D 1.25 nM | 89.65 ± 9.52 |
| H4H14639D 10 nM | 95.02 ± 7.51 |
| H4H14639D-Maytansinoid B 1.25 nM | 94.36 ± 4.61 |
| H4H14639D-Maytansinoid B 10 nM | 86.93 ± 3.95 |

### Example 30: MET×MET Bispecific Antibody ADC Induces Apoptosis in Uveal Melanoma Cells

Uveal melanoma cells that express c-Met, OMM1.3 and Mel202, as well as the c-Met negative cell line, OCM3, were seeded overnight in 60 mm³ plates at 800,000 cells per plate in RPMI with 10% FBS and incubated at 37° C. with 5% CO₂. The cells were treated with 1.25, 2.5 nM, or 10 nM REGN1945 (isotype control antibody), REGN1945-Maytansinoid A, H4H14639D, or H4H14639D-Maytansinoid B for 48 hours. Cell were then harvested with trypsin, washed with PBS, fixed with 4% paraformaldehyde for 30 minutes at room temperature, and stained with DAPI overnight at 4° C. The cells were placed on a microscope slide and sealed with Cytoseal 40. Apoptotic cells were quantified under a microscope with UV light to excite DAPI fluorescence.

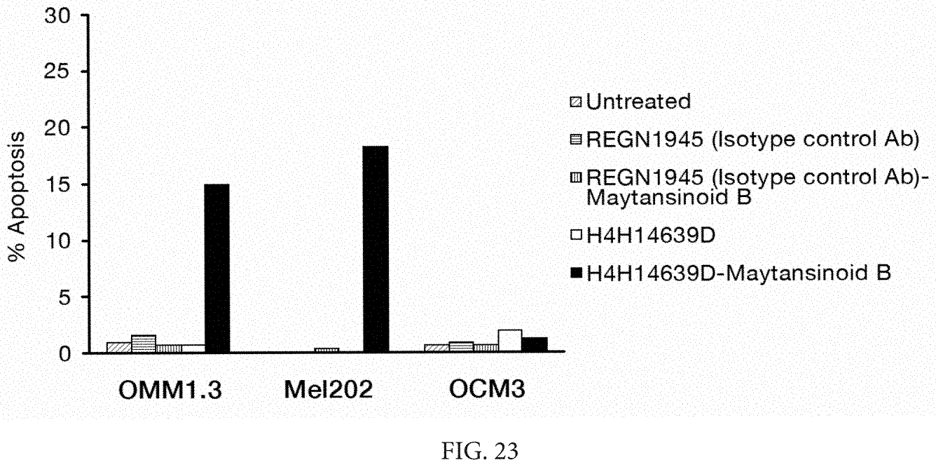

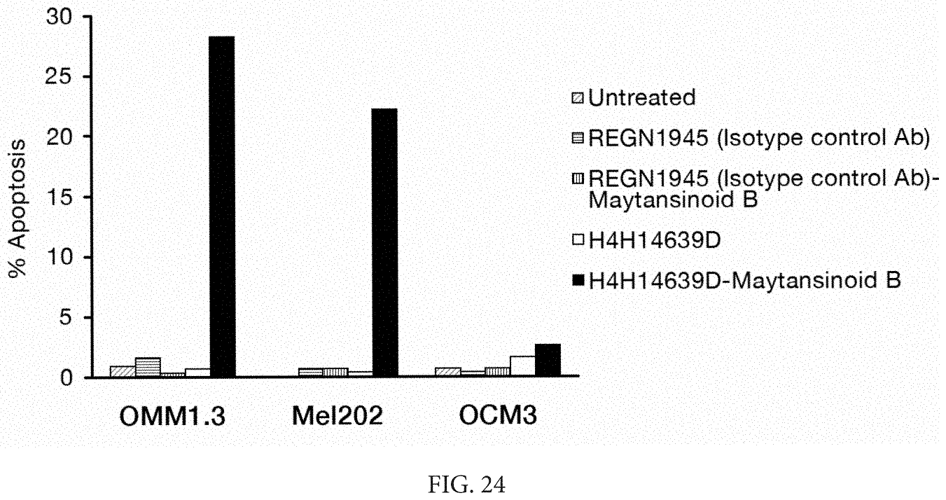

The bispecific c-Met antibody conjugated to a maytansinoid payload, H4H14639D-Maytansinoid B, significantly induced apoptosis of uveal melanoma cells that express the c-Met protein in a dose-dependent manner (see Tables 39 and 40) relative to the control treatments and c-Met negative cell line (see Table 41). See also FIGS. 23 and 24. In another experiment, apoptosis was induced up to 40% in c-Met-expressing cell lines, OMM1.3 and Mel202, but not OCM3 when treated with 10 nM of the MET×MET-ADC for 48 hours (data not shown). By conjugating a c-Met-specific antibody with a cytotoxic compound, uveal melanoma cells can be selectively targeted for apoptosis.

TABLE 39
Apoptosis induced by H4H14639D-
Maytansinoid B in OMM1.3 cells

| OMM1.3 | % apoptosis (n = 1) |
|---|---|
| Untreated | 1 |
| REGN1945 1.25 nM | 1.67 |
| REGN1945 2.5 nM | 1.67 |
| REGN1945-Maytansinoid B 1.25 nM | 0.67 |
| REGN1945-Maytansinoid B 2.5 nM | 0.33 |
| H4H14639D 1.25 nM | 0.67 |
| H4H14639D 2.5 nM | 0.67 |
| H4H14639D-Maytansinoid B 1.25 nM | 15.00 |
| H4H14639D-Maytansinoid B 2.5 nM | 28.33 |

TABLE 40
Apoptosis induced by H4H14639D-
Maytansinoid B in Mel202 cells

| Mel202 | % apoptosis (n = 1) |
|---|---|
| Untreated | 0.00 |
| REGN1945 1.25 nM | 0.00 |
| REGN1945 2.5 nM | 0.67 |
| REGN1945-Maytansinoid B 1.25 nM | 0.33 |
| REGN1945-Maytansinoid B 2.5 nM | 0.67 |
| H4H14639D 1.25 nM | 0.00 |
| H4H14639D 2.5 nM | 0.33 |
| H4H14639D-Maytansinoid B 1.25 nM | 18.33 |
| H4H14639D-Maytansinoid B 2.5 nM | 22.33 |

TABLE 41
Apoptosis induced by H4H14639D-Maytansinoid B in OCM3 cells

| OCM3 | % apoptosis (n = 1) |
|---|---|
| Untreated | 0.67 |
| REGN1945 1.25 nM | 1.00 |
| REGN1945 2.5 nM | 0.33 |
| REGN1945-Maytansinoid B 1.25 nM | 0.67 |
| REGN1945-Maytansinoid B 2.5 nM | 0.67 |
| H4H14639D 1.25 nM | 2.00 |
| H4H14639D 2.5 nM | 1.67 |
| H4H14639D-Maytansinoid B 1.25 nM | 1.33 |
| H4H14639D-Maytansinoid B 2.5 nM | 2.67 |

### Example 31: MET×MET Bispecific Antibody ADC Alters Cell Cycle in Uveal Melanoma Cells

Uveal melanoma cells that express c-Met, OMM1.3 and Mel202, as well as the c-Met negative cell line, OCM3, were seeded overnight in 60 mm³ plates at 800,000 cells per plate in RPMI with 10% FBS and incubated at 37° C. with 5% CO₂. The cells were either untreated or treated with 10 nM H4H14639D-Maytansinoid B for 1, 3, 6, 24 and 48 hours. Cell were then harvested with trypsin, washed with PBS, fixed with cold 70% ethanol overnight at −20° C., incubated in Millipore anti-MPM2 antibody for 2 hours, washed with PBS, incubated in Invitrogen anti-mouse IgG conjugated with Alexa Fluor 488 (Invitrogen) and washed again with PBS. The cells were then stained with 500 μg/ml propidium iodide and incubated overnight at 4° C. The cells were then passed through a cell-strainer before running through the BD Bioscience LSR II flow cytometer. Data was analyzed using FCS Express 6 by De Novo software.

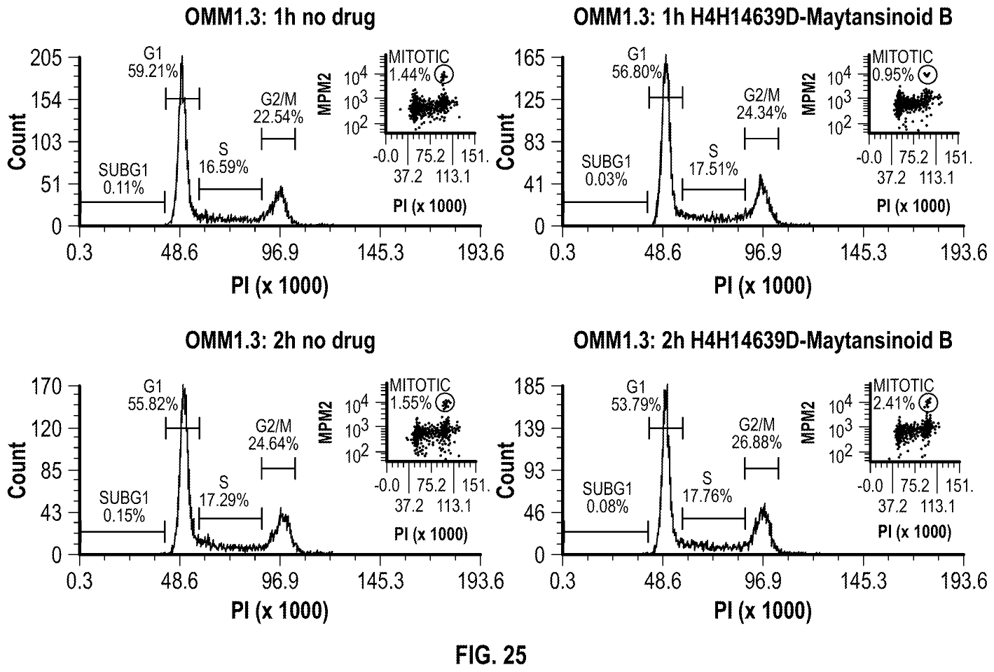

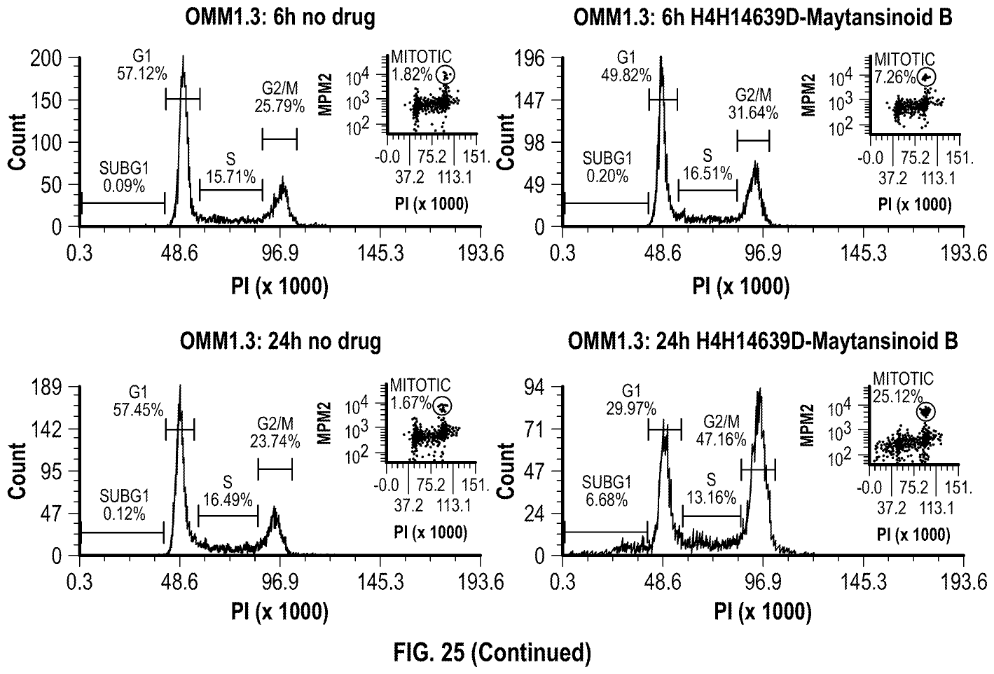

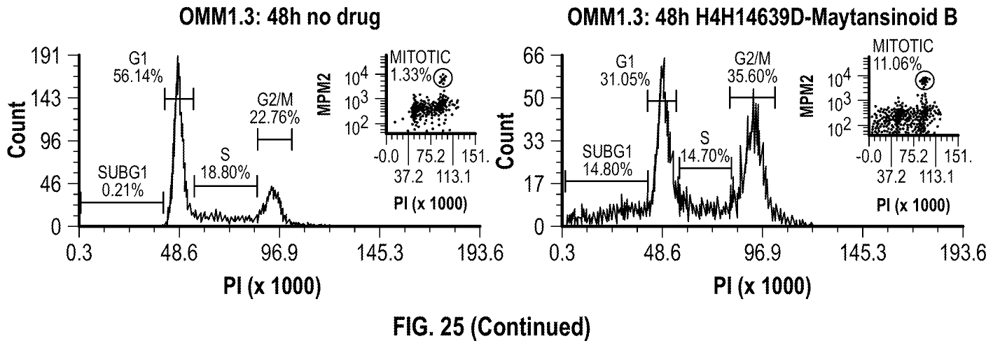

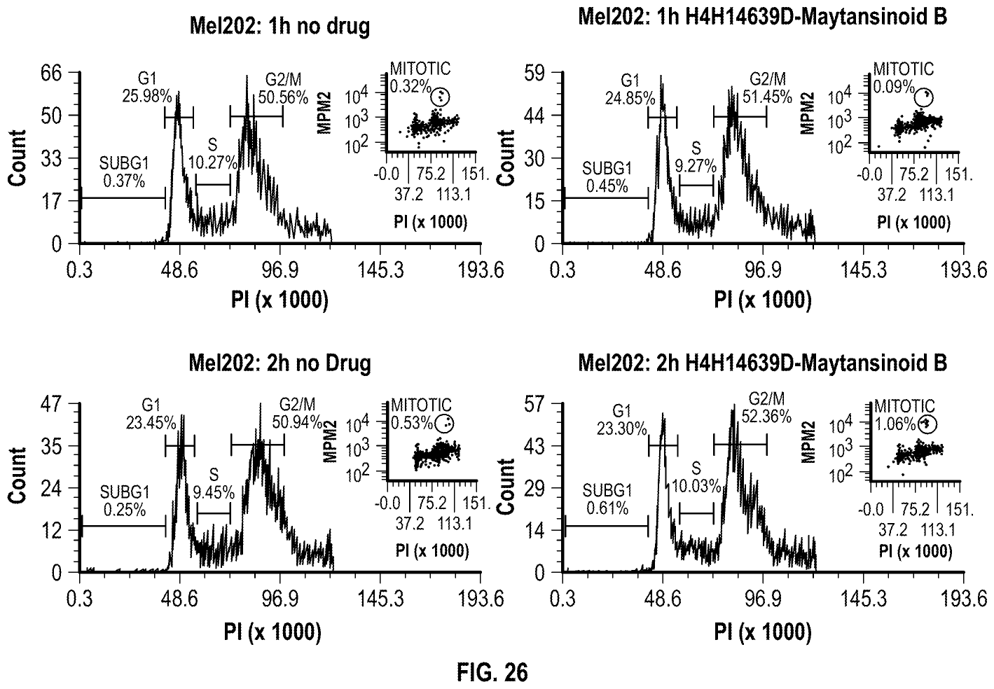

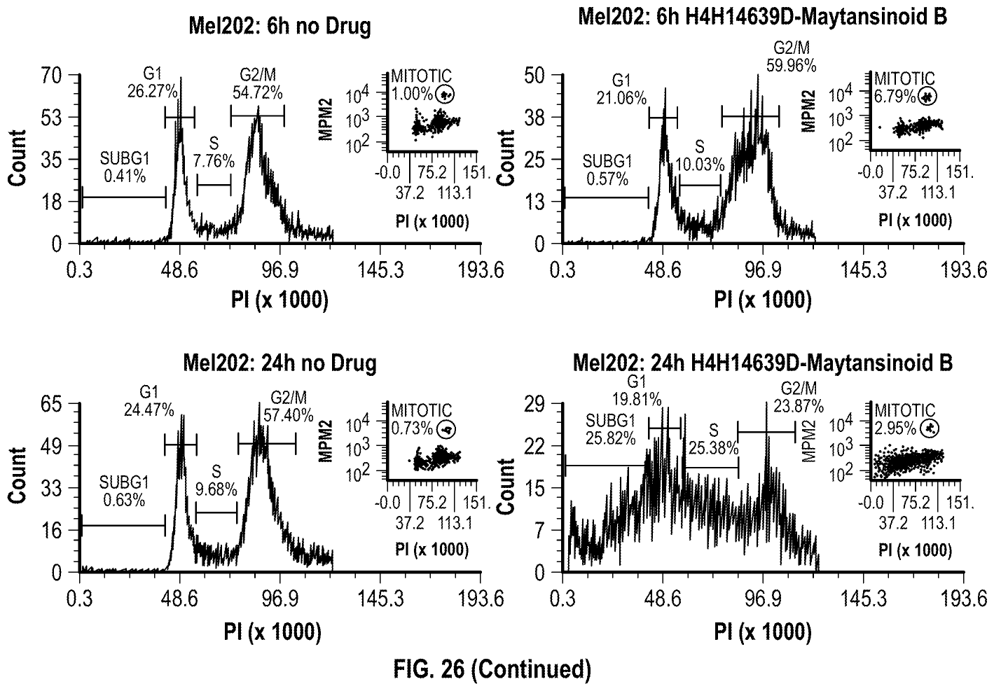

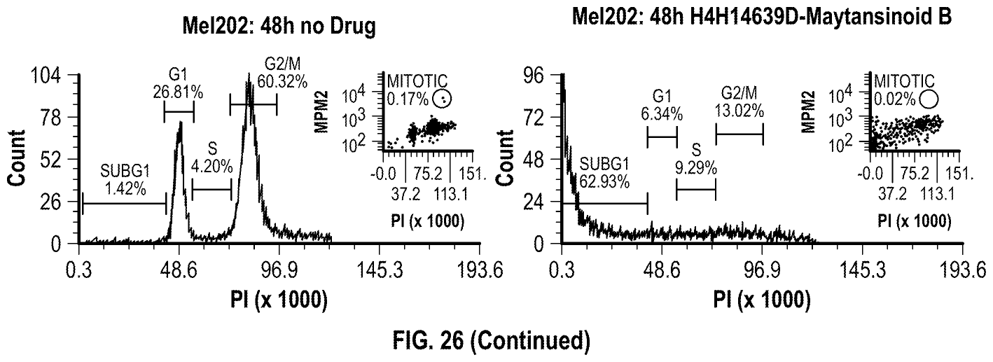

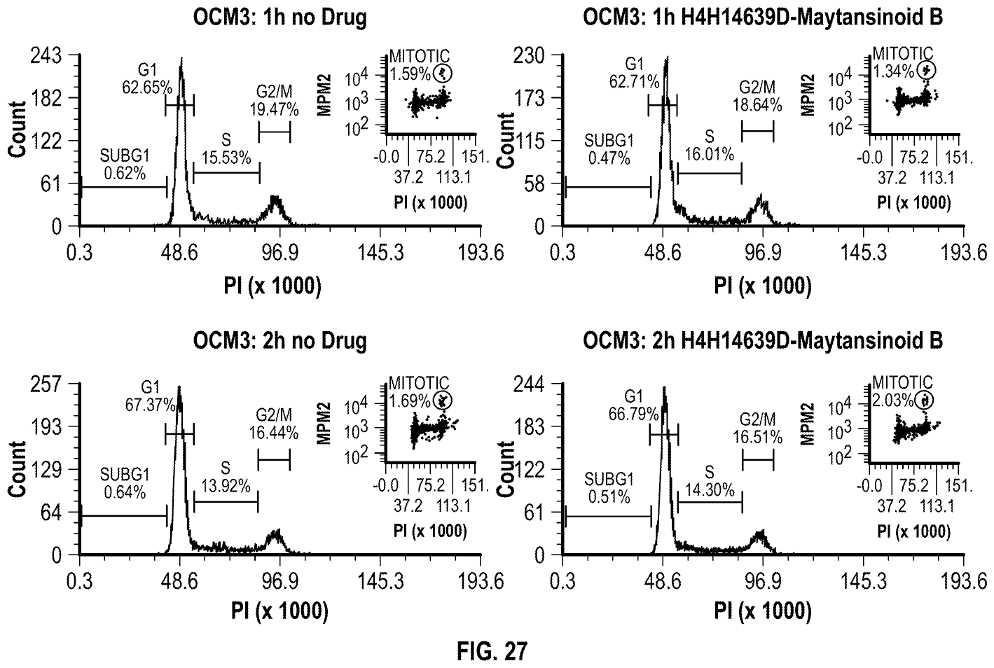

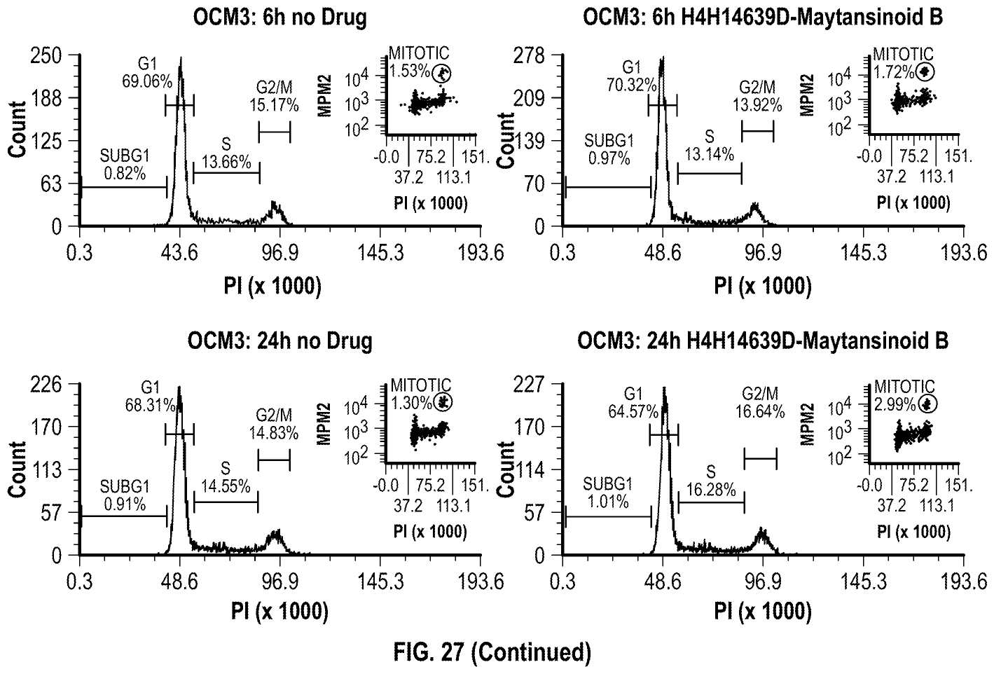

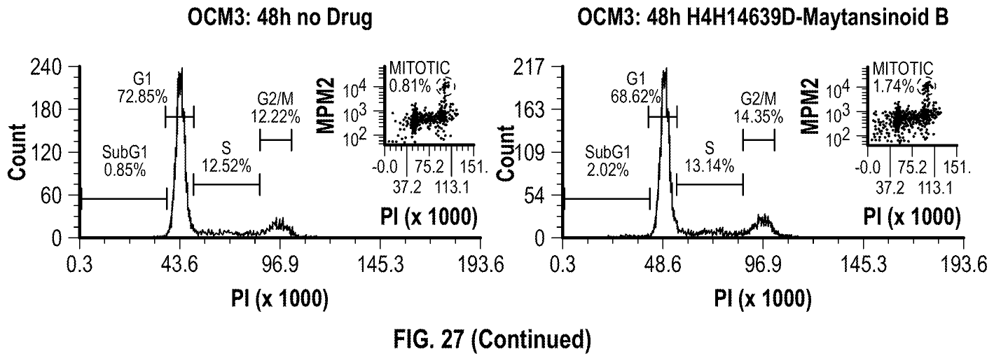

The bispecific c-Met antibody conjugated to a maytansinoid payload, H4H14639D-Maytansinoid B, significantly induced mitotic arrest in OMM1.3 and Mel202 cells (FIGS. 25 and 26, respectively) after 6-24 hours of treatment but did not induce mitotic arrest in OCM3 cells (FIG. 27). There was an increase in the SubG1 population in the c-Met expressing cells treated with H4H14639D-Maytansinoid B between 24-48 hours indicating induction of apoptosis, but no increase was seen in the SubG1 population in c-Met negative cells. See Tables 42-44. The cell cycle analysis confirmed that introduction of the maytansinoid payload induced mitotic arrest and consequently apoptosis only in c-Met expressing cell lines, OMM1.3 and Mel202, and not the c-Met negative OCM3 cell line.

TABLE 42
OMM1.3 cell cycle distribution following 24
hours H4H14639D-Maytansinoid B treatment

| | | | |
|---|---|---|---|
| | % Cells | H4H14639D- | % Cells |
| Untreated | (n = 1) | Maytansinoid B | (n = 1) |

| | |
|---|---|
| SubG1 | SubG1 |

| | | | | | |
|---|---|---|---|---|---|
| 1 | hr | 0.11 | 1 | hr | 0.03 |
| 2 | hr | 0.15 | 2 | hr | 0.08 |
| 6 | hr | 0.09 | 6 | hr | 0.20 |
| 24 | hr | 0.12 | 24 | hr | 6.68 |
| 48 | hr | 0.21 | 48 | hr | 14.80 |

| | |
|---|---|
| G1 | G1 |

| | | | | | |
|---|---|---|---|---|---|
| 1 | hr | 59.21 | 1 | hr | 56.80 |
| 2 | hr | 55.82 | 2 | hr | 53.79 |
| 6 | hr | 57.12 | 6 | hr | 49.82 |
| 24 | hr | 57.45 | 24 | hr | 29.97 |
| 48 | hr | 56.14 | 48 | hr | 31.05 |

| | |
|---|---|
| S | S |

| | | | | | |
|---|---|---|---|---|---|
| 1 | hr | 16.59 | 1 | hr | 17.51 |
| 2 | hr | 17.29 | 2 | hr | 17.76 |
| 6 | hr | 15.71 | 6 | hr | 16.51 |
| 24 | hr | 16.49 | 24 | hr | 13.16 |
| 48 | hr | 18.80 | 48 | hr | 14.70 |

| | |
|---|---|
| G2/M | G2/M |

| | | | | | |
|---|---|---|---|---|---|
| 1 | hr | 22.54 | 1 | hr | 24.34 |
| 2 | hr | 24.64 | 2 | hr | 26.99 |
| 6 | hr | 25.79 | 6 | hr | 31.64 |
| 24 | hr | 23.74 | 24 | hr | 47.16 |
| 48 | hr | 22.76 | 48 | hr | 35.60 |

| | |
|---|---|
| M | M |

| | | | | | |
|---|---|---|---|---|---|
| 1 | hr | 1.44 | 1 | hr | 0.95 |
| 2 | hr | 1.55 | 2 | hr | 2.41 |
| 6 | hr | 1.82 | 6 | hr | 7.26 |
| 24 | hr | 1.67 | 24 | hr | 25.12 |
| 48 | hr | 1.33 | 48 | hr | 11.06 |

TABLE 43
Mel202 cell cycle distribution following 24
hours H4H14639D-Maytansinoid B treatment

| | | | |
|---|---|---|---|
| | % Cells | H4H14639D- | % Cells |
| Untreated | (n = 1) | Maytansinoid B | (n = 1) |

| | |
|---|---|
| SubG1 | SubG1 |

| | | | | | |
|---|---|---|---|---|---|
| 1 | hr | 0.37 | 1 | hr | 0.45 |
| 2 | hr | 0.25 | 2 | hr | 0.61 |
| 6 | hr | 0.41 | 6 | hr | 0.57 |
| 24 | hr | 0.63 | 24 | hr | 25.82 |
| 48 | hr | 1.42 | 48 | hr | 62.93 |

| | |
|---|---|
| G1 | G1 |

| | | | | | |
|---|---|---|---|---|---|
| 1 | hr | 25.98 | 1 | hr | 24.85 |
| 2 | hr | 23.45 | 2 | hr | 23.30 |
| 6 | hr | 26.27 | 6 | hr | 21.06 |
| 24 | hr | 24.47 | 24 | hr | 19.81 |
| 48 | hr | 26.81 | 48 | hr | 6.34 |

| | |
|---|---|
| S | S |

| | | | | | |
|---|---|---|---|---|---|
| 1 | hr | 10.27 | 1 | hr | 9.27 |
| 2 | hr | 9.45 | 2 | hr | 10.03 |
| 6 | hr | 7.76 | 6 | hr | 10.03 |
| 24 | hr | 9.68 | 24 | hr | 25.38 |
| 48 | hr | 4.20 | 48 | hr | 9.29 |

| | |
|---|---|
| G2/M | G2/M |

| | | | | | |
|---|---|---|---|---|---|
| 1 | hr | 50.56 | 1 | hr | 52.45 |
| 2 | hr | 50.94 | 2 | hr | 52.36 |
| 6 | hr | 54.72 | 6 | hr | 59.96 |
| 24 | hr | 57.40 | 24 | hr | 23.87 |
| 48 | hr | 60.32 | 48 | hr | 13.02 |

| | |
|---|---|
| M | M |

| | | | | | |
|---|---|---|---|---|---|
| 1 | hr | 0.32 | 1 | hr | 0.09 |
| 2 | hr | 0.53 | 2 | hr | 1.06 |
| 6 | hr | 1.00 | 6 | hr | 6.79 |
| 24 | hr | 0.73 | 24 | hr | 2.95 |
| 48 | hr | 0.17 | 48 | hr | 0.02 |

TABLE 44
OCM3 cell cycle distribution following 24
hours H4H14639D-Maytansinoid B treatment

| | | | |
|---|---|---|---|
| | % Cells | H4H14639D- | % Cells |
| Untreated | (n = 1) | Maytansinoid B | (n = 1) |

| | |
|---|---|
| SubG1 | SubG1 |

| | | | | | |
|---|---|---|---|---|---|
| 1 | hr | 0.62 | 1 | hr | 0.47 |
| 2 | hr | 0.64 | 2 | hr | 0.51 |
| 6 | hr | 0.82 | 6 | hr | 0.97 |
| 24 | hr | 0.91 | 24 | hr | 1.01 |
| 48 | hr | 0.85 | 48 | hr | 2.02 |

| | |
|---|---|
| G1 | G1 |

| | | | | | |
|---|---|---|---|---|---|
| 1 | hr | 62.65 | 1 | hr | 62.71 |
| 2 | hr | 67.37 | 2 | hr | 66.79 |
| 6 | hr | 69.06 | 6 | hr | 70.32 |
| 24 | hr | 68.31 | 24 | hr | 64.57 |
| 48 | hr | 72.85 | 48 | hr | 68.62 |

| | |
|---|---|
| S | S |

| | | | | | |
|---|---|---|---|---|---|
| 1 | hr | 15.53 | 1 | hr | 16.01 |
| 2 | hr | 13.92 | 2 | hr | 14.30 |
| 6 | hr | 13.66 | 6 | hr | 13.41 |
| 24 | hr | 14.55 | 24 | hr | 16.28 |
| 48 | hr | 12.52 | 48 | hr | 13.14 |

| | |
|---|---|
| G2/M | G2/M |

| | | | | | |
|---|---|---|---|---|---|
| 1 | hr | 19.47 | 1 | hr | 18.64 |
| 2 | hr | 16.44 | 2 | hr | 16.51 |
| 6 | hr | 15.17 | 6 | hr | 13.92 |
| 24 | hr | 14.83 | 24 | hr | 16.64 |
| 48 | hr | 12.22 | 48 | hr | 14.35 |

| | |
|---|---|
| M | M |

| | | | | | |
|---|---|---|---|---|---|
| 1 | hr | 1.59 | 1 | hr | 1.34 |
| 2 | hr | 1.69 | 2 | hr | 2.03 |
| 6 | hr | 1.53 | 6 | hr | 1.72 |
| 24 | hr | 1.30 | 24 | hr | 2.99 |
| 48 | hr | 0.81 | 48 | hr | 1.74 |

### Example 32: c-Met Expression in Uveal Melanoma Cell Lines

Western blot analyses were performed to assess differences in c-Met protein expression levels in several uveal melanoma cell lines as well as a gastric carcinoma cell line and a lung carcinoma cell line.

Cell lines with varying levels of c-Met expression including SNU-5, a gastric carcinoma cell line, A549, a lung carcinoma cell line, as well as uveal melanoma cell lines, Mel290, 92.1, OMM1.3, OMM1, Mel285, Mel202, Mel270, OCM1A, OCM3, MP41, MP65, MP46 and UM004, were plated on 60 mm³ plates at 1,000,000 cells per pate in RPMI with 10% FBS and incubated at 37° C. with 5% CO₂ for 24 hours. The cells were then harvested with trypsin, washed with PBS, and lysed with RIPA buffer. Protein lysates were run on 20-well Novex midi gels 4-12% (Invitrogen) then transferred on a PVDF membrane. The membrane was then blocked in 5% non-fat dry milk, incubated in primary antibodies against c-Met (Cell Signaling) and tubulin (Cell Signaling) overnight on a shaker at 4° C., washed with TBST, incubated in the appropriate secondary antibodies (GE Healthcare) conjugated with HRP and washed with TBST. ECL HRP substrate was added onto the membrane and the fluorescence image was taken using Fujifilm XA-2 camera.

### Results

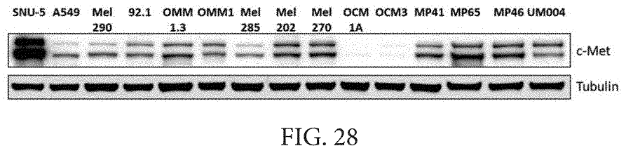

Uveal melanoma cell lines are commonly noted for mutations in G proteins such as GNAQ or GNA11, but they also exhibit differential c-Met expression. As shown in FIG. 28, each of the uveal melanoma cell lines express the c-Met receptor at some level, except for the OCM1A and OCM3 cell lines, which happen to be BRAF^{V600E-} mutant cells. SNU-5 is a positive control gastric carcinoma cell line known to highly express c-Met while A549 is a lung carcinoma cell line that also expresses c-Met.

### Example 33: MET×MET Bispecific Antibody ADC Induces PARP Cleavage and Histone H3 Phosphorylation

Western blot analyses were performed to assess c-Met protein levels, PARP cleavage and histone H3 phosphorylation in several uveal melanoma cell lines after treatment with H4H14639D-Maytansinoid B.

Uveal melanoma cells that express c-Met, OMM1.3 and Mel202, as well as c-Met negative cell line, OCM3, were seeded overnight in 60 mm³ plates at 800,000 cells per plate in RPMI with 10% FBS and incubated at 37° C. with 5% CO₂. The cells were either untreated or treated with increasing doses of REGN1945-Maytansinoid B, H4H14639D and H4H14639D-Maytansinoid B from 0.5 to 10 nM for 24 hours. The cells were then harvested with trypsin, washed with PBS, and lysed with RIPA buffer. Protein lysates were run on 20-well Novex midi gels 4-12% (Invitrogen) then transferred on a PVDF membrane. The membrane was then blocked in 5% non-fat dry milk, incubated in primary antibodies against PARP (Cell Signaling), phosphorylated histone-H3 (Cell Signaling), and tubulin (Cell Signaling) overnight on a shaker at 4° C., washed with TBST, incubated in the appropriate secondary antibodies (GE Healthcare) conjugated with HRP and washed with TBST. ECL HRP substrate was added onto the membrane and the fluorescence image was taken using Fujifilm XA-2 camera.

In another experiment, uveal melanoma cells that express c-Met, OMM1.3, as well as a c-Met negative cell line, OCM3, were seeded overnight in 60 mm³ plates at 800,000 cells per plate in RPMI with 10% FBS and incubated at 37° C. with 5% CO₂. However, in this experiment, the cells were either untreated or treated with 10 nM REGN1945-Maytansinoid B, H4H14639D and H4H14639D-Maytansinoid B for the longer time periods of 24, 48 and 72 hours. The cells were then harvested with trypsin, washed with PBS, and lysed with RIPA buffer. Protein lysates were run on 20-well Novex midi gels 4-12% (Invitrogen) then transferred on a PVDF membrane. The membrane was then blocked in 5% non-fat dry milk, incubated in primary antibodies against c-Met (Cell Signaling), PARP (Cell Signaling), phosphorylated histone-H3 (Cell Signaling), and tubulin (Cell Signaling) overnight on a shaker at 4° C., washed with TBST, incubated in the appropriate secondary antibodies (GE Healthcare) conjugated with HRP and washed with TBST. ECL HRP substrate was added onto the membrane and the fluorescence image was taken using Fujifilm XA-2 camera.

### Results

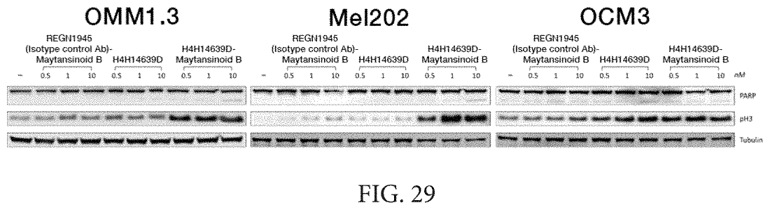

FIG. 29 is an image of a Western blot showing that H4H14639D-Maytansinoid B induces PARP cleavage (a marker of apoptosis) in OMM1.3 cells and Mel202 cells after 24 hours of treatment. Neither REGN1945-Maytansinoid B nor H4H14639D induced PARP cleavage. Unlike the c-Met positive cell lines, OCM3 cells did not exhibit PARP cleavage after H4H14639D-Maytansinoid B treatment. FIG. 29 also shows a significant increase in histone H3 phosphorylation in OMM1.3 and Mel202 cells treated with H4H14639D-Maytansinoid B compared to REGN1945-Maytansinoid B and H4H14639D, but not in OCM3 cells. Histone H3 phosphorylation is induced during mitosis and is evidence of the maytansinoid-induced mitotic arrest in the cell. In FIG. 29, histone H3 phosphorylation is seen only in c-Met expressing cells (OMM1.3 and Mel202) and not OCM3 and is evidence that the maytansinoid is transported into the cell by the c-Met antibody. This data further demonstrates specificity and effectiveness of the c-Met ADC.

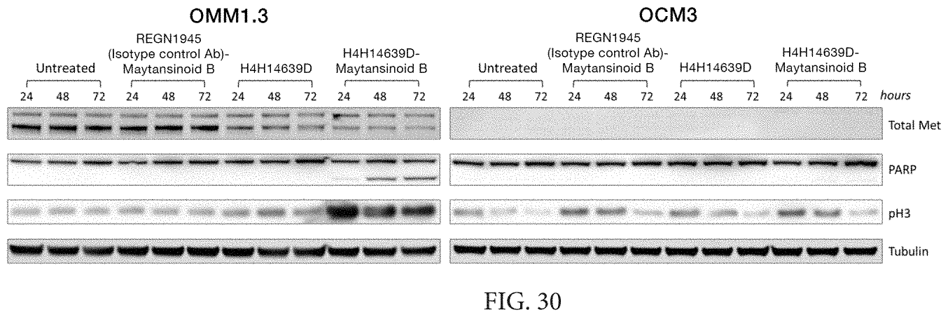

FIG. 30 is an image of a Western blot showing a time-dependent induction of PARP cleavage in H4H14639D-Maytansinoid B-treated OMM1.3 cells but not in REGN1945-Maytansinoid B or H4H14639D-treated OMM1.3 cells. PARP protein was not affected by H4H14639D-Maytansinoid B treatment in OCM3 cells. In addition, total Met protein expression is decreased when treated with H4H14639D and H4H14639D-M114 compared to untreated and REGN1945-M114, indicating receptor internalization after treatment with the antibody or ADC. Lastly, there was a significant increase in histone H3 phosphorylation in OMM1.3 cells treated with H4H14639D-Maytansinoid B (compared to treatment with REGN1945-Maytansinoid B or H4H14639D), but again, that increase was not observed in OCM3 cells.

In conclusion, an exemplary bispecific anti-c-Met antibody, the H4H14639D antibody, specifically targets c-Met in cells expressing this receptor. By conjugating this antibody with a maytansinoid (H4H14639D-Maytansinoid B), apoptosis can be specifically and potently induced in uveal melanoma cell lines that express c-Met.

### Example 34: MET×MET Bispecific Antibody ADC Inhibits Invasion of c-Met Expressing Uveal Melanoma Cells

Uveal melanoma cells that express c-Met, OMM1.3, were seeded overnight in matrigel inserts placed in a 24-well plate at 120,000 cells per insert in RPMI with 0.1% FBS with the following treatments: untreated control, 125, 250 and 500 μM R1945, R1945-Maytansinoid B, H4H14639D and H4H14639D-Maytansinoid B. RPMI with 10% FBS and 50 ng/ml human HGF were placed in the well as chemoattractant. After approximately 24 hours, the insert-side of the matrigel was cleaned of non-migrated cells. The migrated cells were fixed with methanol for 2 minutes and stained with 1% toluidine for 2 minutes and then washed twice with ddH₂O. The dried matrigels were then placed on microscope slides and sealed with Cytoseal 60. Images were taken using Nikon TE-2000-U microscope.

### Results

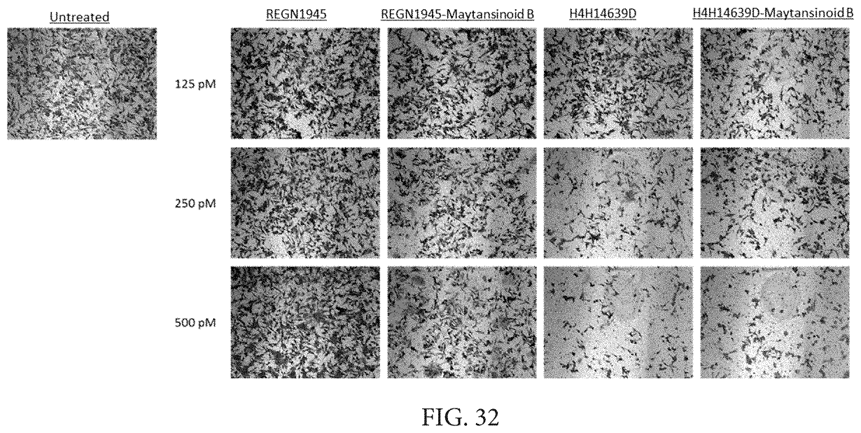

The bispecific c-Met antibody H4H14639D-Maytansinoid B significantly inhibited invasion of OMM1.3 uveal melanoma cells that express the c-Met protein relative to the control treatments (R1945 and R1945-Maytansinoid B) starting at 250 μM. There was also significant inhibition of cell invasion in cells treated with H4H14639D starting at 250 μM. Cell viability, however, is not affected by the conjugated maytansinoid payload in H4H14639D-Maytansinoid B at this dose. See FIG. 32.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method of treating an uveal melanoma, reducing uveal melanoma tumor growth, and/or causing regression of an uveal melanoma in a subject, the method comprising administering to a subject in need thereof an antibody-drug conjugate (ADC) comprising a bispecific antigen-binding molecule and a cytotoxin, wherein the bispecific antigen-binding molecule comprises:
a first antigen-binding domain (D1); and
a second antigen-binding domain (D2);
wherein D1 specifically binds a first epitope of human MET; and
wherein D2 specifically binds a second epitope of human MET.

2. The method of claim 1, wherein the uveal melanoma expresses MET.

3. The method of claim 1, wherein D1 comprises three heavy chain complementarity determining regions (HCDR1, HCDR2 and HCDR3) within a heavy chain variable region (HCVR) comprising the amino acid sequence of SEQ ID NO:58 and three light chain complementarity determining regions (LCDR1, LCDR2 and LCDR3) within a light chain variable region (LCVR) comprising the amino acid sequence of SEQ ID NO:138.

4. The method of claim 1, wherein D2 comprises three heavy chain complementarity determining regions (HCDR1, HCDR2 and HCDR3) within a heavy chain variable region (HCVR) comprising the amino acid sequence of SEQ ID NO:82 and three light chain complementarity determining regions (LCDR1, LCDR2 and LCDR3) within a light chain variable region (LCVR) comprising the amino acid sequence of SEQ ID NO:138.

5. The method of claim 1, wherein the bispecific antigen-binding molecule comprises the CDRs within the D1-HCVR amino acid sequence of SEQ ID NO: 58 and the CDRs within the D2-HCVR amino acid sequence of SEQ ID NO: 82.

6. The method of claim 1, wherein the first epitope of human MET comprises amino acids 192-204 of SEQ ID NO:155.

7. The method of claim 1, wherein the second epitope of human MET comprises amino acids 305-315 and 421-455 of SEQ ID NO:155.

8. The method of claim 1, wherein the first epitope of human MET comprises amino acids 192-204 of SEQ ID NO:155; and wherein the second epitope of human MET comprises amino acids 305-315 and 421-455 of SEQ ID NO:155.

9. The method of claim 1, wherein the cytotoxin is selected from the group consisting of biotoxins, chemotherapeutic agents, and radioisotopes.

10. The method of claim 1, wherein the cytotoxin is selected from the group consisting of maytansinoids, auristatins, tomaymycins, duocarmycins, ²²⁵AC, ²²⁷Th, and any derivatives thereof.

11. The method of claim 1, wherein the cytotoxin is conjugated to the bispecific antigen-binding molecule through a linker, and wherein the cytotoxin is: 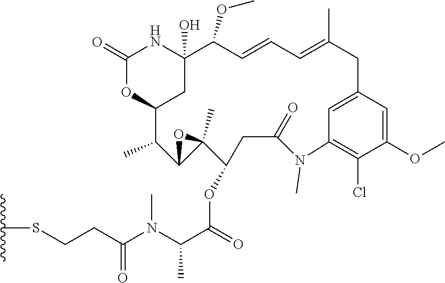 [SMILES: *SCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]
wherein the [symbol] is the bond to a linker.

12. The method of claim 11, wherein the linker is: 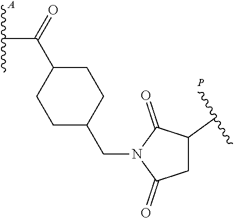
wherein the bond noted with [symbol] represents the bond to the bispecific antigen-binding molecule and the bond noted with [symbol] represents the bond to the cytotoxin.

13. The method of claim 1, wherein the cytotoxin is conjugated to the bispecific antigen-binding molecule through a linker, and wherein the cytotoxin is: 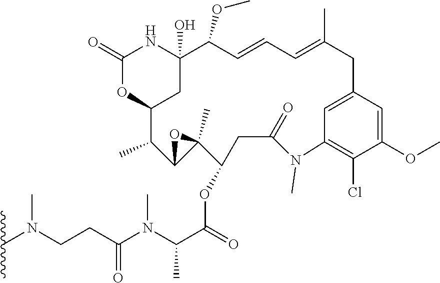 [SMILES: *N(C)CCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]
wherein the [symbol] is the bond to the linker.

14. The method of claim 13, wherein the linker is 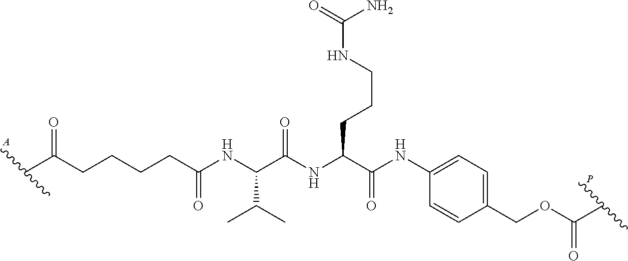
wherein the bond noted with [symbol] represents the bond to the bispecific antigen-binding molecule and the bond noted with [symbol] represents the bond to the cytotoxin.

15. A method of inhibiting proliferation, inhibiting invasion, causing apoptosis, and/or decreasing viability of a uveal melanoma cell, the method comprising contacting the cell with an antibody-drug conjugate (ADC) comprising a bispecific antigen-binding molecule and a cytotoxin, wherein the bispecific antigen-binding molecule comprises:
a first antigen-binding domain (D1); and
a second antigen-binding domain (D2);
wherein D1 specifically binds a first epitope of human MET; and
wherein D2 specifically binds a second epitope of human MET.

16. The method of claim 15, wherein D1 comprises three heavy chain complementarity determining regions (HCDR1, HCDR2 and HCDR3) within a heavy chain variable region (HCVR) comprising the amino acid sequence of SEQ ID NO:58 and three light chain complementarity determining regions (LCDR1, LCDR2 and LCDR3) within a light chain variable region (LCVR) comprising the amino acid sequence of SEQ ID NO:138.

17. The method of claim 15, wherein D2 comprises three heavy chain complementarity determining regions (HCDR1, HCDR2 and HCDR3) within a heavy chain variable region (HCVR) comprising the amino acid sequence of SEQ ID NO:82 and three light chain complementarity determining regions (LCDR1, LCDR2 and LCDR3) within a light chain variable region (LCVR) comprising the amino acid sequence of SEQ ID NO:138.

18. The method of claim 15, wherein the cytotoxin is conjugated to the bispecific antigen-binding molecule through a linker, and wherein the cytotoxin is: 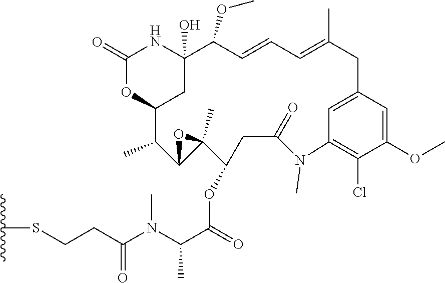 [SMILES: *SCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]
wherein the [symbol] is the bond to a linker.

19. The method of claim 18, wherein the linker is: 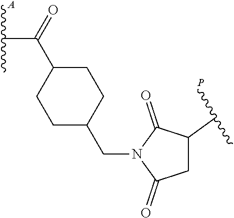
wherein the bond noted with [symbol] represents the bond to the bispecific antigen-binding molecule and the bond noted with [symbol] represents the bond to the cytotoxin.

20. The method of claim 15, wherein the cytotoxin is conjugated to the bispecific antigen-binding molecule through a linker, and wherein the cytotoxin is: 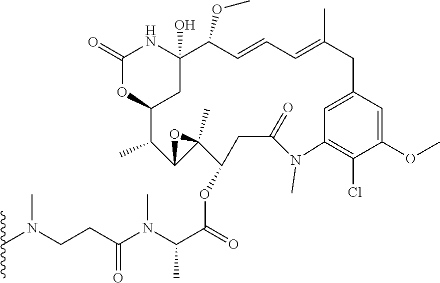 [SMILES: *N(C)CCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]
wherein the [symbol] is the bond to the linker.

21. The method of claim 20, wherein the linker is 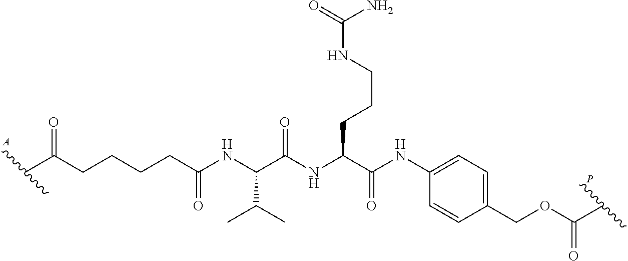
wherein the bond noted with [symbol] represents the bond to the bispecific antigen-binding molecule and the bond noted with [symbol] represents the bond to the cytotoxin.

22. A method of inducing mitotic arrest of an uveal melanoma cell, the method comprising contacting the cell in vivo with an antibody-drug conjugate (ADC) comprising a bispecific antigen-binding molecule and a cytotoxin, wherein the bispecific antigen-binding molecule comprises:
a first antigen-binding domain (D1); and
a second antigen-binding domain (D2);
wherein D1 specifically binds a first epitope of human MET; and
wherein D2 specifically binds a second epitope of human MET.

23. The method of claim 22, wherein D1 comprises three heavy chain complementarity determining regions (HCDR1, HCDR2 and HCDR3) within a heavy chain variable region (HCVR) comprising the amino acid sequence of SEQ ID NO:58 and three light chain complementarity determining regions (LCDR1, LCDR2 and LCDR3) within a light chain variable region (LCVR) comprising the amino acid sequence of SEQ ID NO:138.

24. The method of claim 22, wherein D2 comprises three heavy chain complementarity determining regions (HCDR1, HCDR2 and HCDR3) within a heavy chain variable region (HCVR) comprising the amino acid sequence of SEQ ID NO:82 and three light chain complementarity determining regions (LCDR1, LCDR2 and LCDR3) within a light chain variable region (LCVR) comprising the amino acid sequence of SEQ ID NO:138.

25. The method of claim 22, wherein the cytotoxin is conjugated to the bispecific antigen-binding molecule through a linker, and wherein the cytotoxin is: 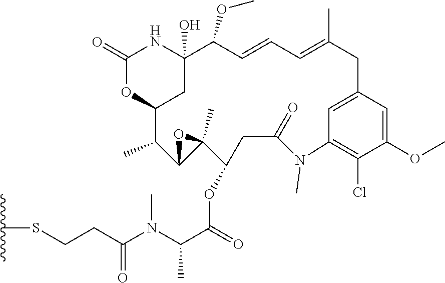 [SMILES: *SCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]
wherein the [symbol] is the bond to a linker.

26. The method of claim 25, wherein the linker is: 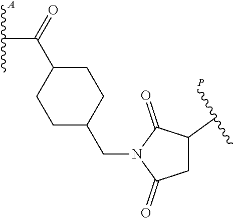
wherein the bond noted with [symbol] represents the bond to the bispecific antigen-binding molecule and the bond noted with [symbol] represents the bond to the cytotoxin.

27. The method of claim 22, wherein the cytotoxin is conjugated to the bispecific antigen-binding molecule through a linker, and wherein the cytotoxin is: 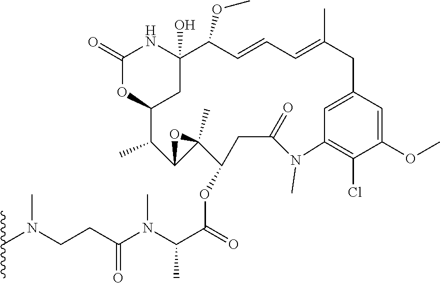 [SMILES: *N(C)CCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]
wherein the [symbol] is the bond to the linker.

28. The method of claim 27, wherein the linker is 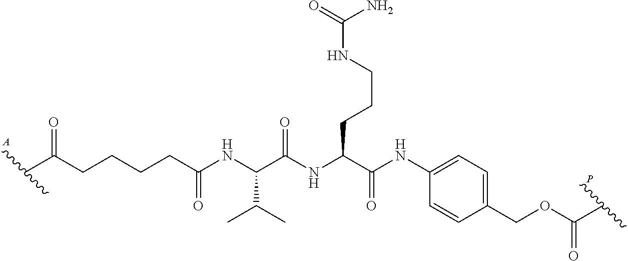
wherein the bond noted with [symbol] represents the bond to the bispecific antigen-binding molecule and the bond noted with [symbol] represents the bond to the cytotoxin.

29. A method of treating eye cancer or inhibiting metastasis in a subject suffering from a c-Met expressing tumor, the method comprising administering to the subject a bispecific antigen-binding molecule comprising:
a first antigen-binding domain (D1); and
a second antigen-binding domain (D2);
wherein D1 specifically binds a first epitope of human MET; and
wherein D2 specifically binds a second epitope of human MET.

30. The method of claim 29, wherein the eye cancer is selected from the group consisting of uveal melanoma, orbital lymphoma, retinoblastoma, and medulloepithelioma.

31. The method of claim 29, further comprising administering to the subject a second anti-cancer therapeutic agent.

32. The method of claim 29, wherein D1 and D2 do not compete with one another for binding to human MET.

33. The method of claim 29, wherein D1 comprises three heavy chain complementarity determining regions (HCDR1, HCDR2 and HCDR3) within a heavy chain variable region (HCVR) comprising the amino acid sequence of SEQ ID NO:58 or an amino acid sequence that is at least 95% identical thereto and three light chain complementarity determining regions (LCDR1, LCDR2 and LCDR3) within a light chain variable region (LCVR) comprising the amino acid sequence of SEQ ID NO:138 or an amino acid sequence that is at least 95% identical thereto.

34. The method of claim 29, wherein HCDR1 comprises the amino acid sequence of SEQ ID NO:60; HCDR2 comprises the amino acid sequence of SEQ ID NO:62; HCDR3 comprises the amino acid sequence of SEQ ID NO:64; LCDR1 comprises the amino acid sequence of SEQ ID NO:140; LCDR2 comprises the amino acid sequence of SEQ ID NO:142; and LCDR3 comprises the amino acid sequence of SEQ ID NO:144.

35. The method of claim 34, wherein the bispecific antigen-binding molecule comprises an HCVR comprising the amino acid sequence of SEQ ID NO:58 or an amino acid sequence that is at least 95% identical thereto; and an LCVR comprising the amino acid sequence of SEQ ID NO:138 or an amino acid sequence that is at least 95% identical thereto.

36. The method of claim 35, wherein the bispecific antigen-binding molecule comprises an HCVR comprising the amino acid sequence of SEQ ID NO:58; and an LCVR comprising the amino acid sequence of SEQ ID NO:138.

37. The method of claim 36, wherein D2 comprises three heavy chain complementarity determining regions (HCDR1, HCDR2 and HCDR3) within a heavy chain variable region (HCVR) comprising the amino acid sequence of SEQ ID NO:82 or an amino acid sequence that is at least 95% identical thereto and three light chain complementarity determining regions (LCDR1, LCDR2 and LCDR3) within a light chain variable region (LCVR) comprising the amino acid sequence of SEQ ID NO:138 or an amino acid sequence that is at least 95% identical thereto.

38. The method of claim 37, wherein HCDR1 comprises the amino acid sequence of SEQ ID NO:84; HCDR2 comprises the amino acid sequence of SEQ ID NO:86; HCDR3 comprises the amino acid sequence of SEQ ID NO:88; LCDR1 comprises the amino acid sequence of SEQ ID NO:140; LCDR2 comprises the amino acid sequence of SEQ ID NO:142; and LCDR3 comprises the amino acid sequence of SEQ ID NO:144.

39. The method of claim 38, wherein the bispecific antigen-binding molecule comprises an HCVR comprising the amino acid sequence of SEQ ID NO:82 or an amino acid sequence that is at least 95% identical thereto; and an LCVR comprising the amino acid sequence of SEQ ID NO:138 or an amino acid sequence that is at least 95% identical thereto.

40. The method of claim 39, wherein the bispecific antigen-binding molecule comprises an HCVR comprising the amino acid sequence of SEQ ID NO:82; and an LCVR comprising the amino acid sequence of SEQ ID NO:138.

41. The method of claim 29, wherein the bispecific antigen-binding molecule is conjugated to a cytotoxin to form an antibody-drug conjugate (ADC), and wherein the cytotoxin is a maytansinoid.
